(19) United States
(12) Patent Application Publication
(10) Pub. No.: US 2007/0254015 A1
(43) Pub. Date: Nov. 1, 2007

(54) Sanitizing surfaces

(75) Inventors: Muriel Y. Ishikawa, Livermore, CA (US); Leif T. Stordal, Kirkland, WA (US); Lowell L. Wood, Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/592,010

(22) Filed: Nov. 1, 2006

Related U.S. Application Data
(63) Continuation-in-part of application No. 11/414,743, filed on Apr. 28, 2006.
Application No. 11/414,743 is a continuation-in-part of application No. 11/440,460, filed on May 23, 2006.

(51) Int. Cl. A61K 9/70

(52) U.S. Cl. 424/443

(57) ABSTRACT

The present disclosure relates to materials and gloves that include one or more sanitizing layers that include one or more substantially continually sanitizing surfaces.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 11/414,743, entitled METHODS AND SYSTEMS FOR MONITORING STERILIZATION STATUS, naming Edward K. Y. Jung, Royce A. Levien, Robert W. Lord, Mark A. Malamud, John D. Rinaldo, Jr., and Lowell L. Wood, Jr. as inventors, filed 28 Apr. 2006, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/440,460, entitled METHODS AND SYSTEMS FOR STERILIZATION, naming Edward K. Y. Jung, Royce A. Levien, Robert W. Lord, Mark A. Malamud, John D. Rinaldo, Jr., and Lowell L. Wood, Jr. as inventors, filed 23 May 2006, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s)from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present disclosure relates to materials and gloves that include one or more sanitizing layers that include one or more substantially continually sanitizing surfaces.

### SUMMARY

In some embodiments one or more materials are provided that include one or more sanitizing layers that include one or more substantially continually sanitizing surfaces and one or more substantially impermeable layers and one or more sanitizing agents that are integrally associated with the one or more sanitizing layers and that are substantially freely releasable from the one or more substantially continually sanitizing surfaces. In addition to the foregoing, other aspects are described in the claims, drawings, and text forming a part of the present disclosure.

In some embodiments one or more gloves are provided that include one or more sanitizing layers that include one or more substantially continually sanitizing surfaces and one or more substantially impermeable layers and one or more sanitizing agents that are integrally associated with the one or more sanitizing layers and that are substantially freely releasable from the one or more substantially continually sanitizing surfaces. In addition to the foregoing, other aspects are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings, claims, and the following detailed description.

### BRIEF DESCRIPTION OF THE FIGURES

FIG. 1 illustrates an example material 100 in which embodiments may be implemented.

FIG. 2 illustrates alternate embodiments of material 100.

FIG. 3 illustrates alternate embodiments of material 100.

FIG. 4 illustrates alternate embodiments of material 100.

FIG. 5 illustrates alternate embodiments of material 100.

FIG. 6 illustrates alternate embodiments of material 100.

FIG. 7 illustrates alternate embodiments of material 100.

FIG. 8 illustrates alternate embodiments of material 100.

FIG. 9 illustrates alternate embodiments of material 100.

FIG. 10 illustrates an example glove 1000 in which embodiments may be implemented.

FIG. 11 illustrates alternate embodiments of glove 1000.

FIG. 12 illustrates alternate embodiments of glove 1000.

FIG. 13 illustrates alternate embodiments of glove 1000.

FIG. 14 illustrates alternate embodiments of glove 1000.

FIG. 15 illustrates alternate embodiments of glove 1000.

FIG. 16 illustrates alternate embodiments of glove 1000.

FIG. 17 illustrates alternate embodiments of glove 1000.

FIG. 18 illustrates a cross-sectional view of an embodiment of a sanitizing layer 110 of material 100.

FIG. 19 illustrates a cross-sectional view of an embodiment of a sanitizing layer 110 of material 100.

FIG. 20 illustrates a cross-sectional view of an embodiment of a sanitizing layer 110 of material 100.

FIG. 21 illustrates a cross-sectional view of an embodiment of a sanitizing layer 110 of material 100.

FIG. 22 illustrates a cross-sectional view of an embodiment of a sanitizing layer 110 of material 100.

FIG. 23 illustrates a cross-sectional view of an embodiment of a sanitizing layer 110 of material 100.

FIG. 24 illustrates a cross-sectional view of an embodiment of a sanitizing layer 110 of material 100.

FIG. 25 illustrates a cross-sectional view of an embodiment of a sanitizing layer 110 of material 100.

FIG. 26 illustrates a cross-sectional view of an embodiment of a sanitizing layer 110 of material 100.

FIG. 27 illustrates a cross-sectional view of an embodiment of a sanitizing layer 110 of material 100.

FIG. 28 illustrates a cross-sectional view of an embodiment of a sanitizing layer 110 of material 100.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

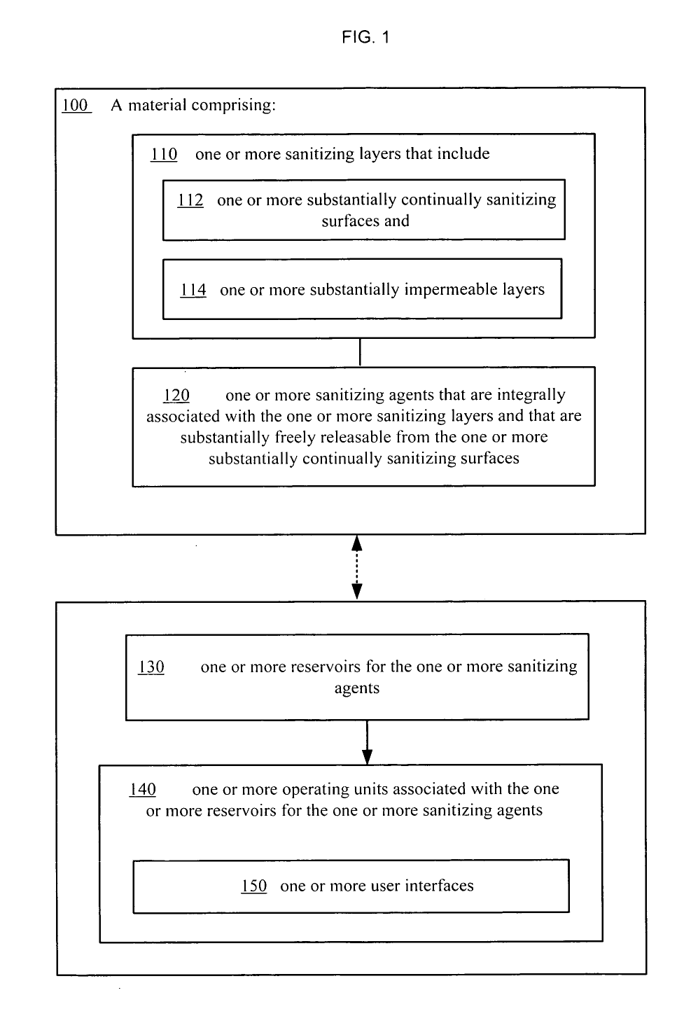

FIG. 1 represents a material 100 that may include one or more sanitizing layers that include one or more substantially continually sanitizing surfaces and one or more substantially impermeable layers.

A sanitizing layer 110 that includes one or more substantially continually sanitizing surfaces 112 is a layer from which one or more sanitizing agents 120 may be substantially freely releasable from the one or more substantially continually sanitizing surfaces 112. In some embodiments, one or more sanitizing agents 120 may be substantially continually freely released until the supply of the sanitizing agents 120 is exhausted. In some embodiments, one or more sanitizing agents 120 may be substantially continually freely released on an intermittent basis. For example, in some embodiments, sanitizing agents 120 may be substantially continually freely released for a period of time, after which the release of the sanitizing agents 120 may be halted for a period of time, and then the release of the sanitizing agent 120 may be resumed. In some embodiments, the one or more sanitizing agents 120 may be prevented from being released from a substantially continually sanitizing surface 112 until release is initiated whereupon the one or more sanitizing agents 120 may be substantially continually freely released.

In some embodiments, a sanitizing layer 110 may include one or more portions that release a first type of sanitizing agent 120 and one or more portions that release a second type of sanitizing agent 120 that is different that the first type of sanitizing agent 120. In some embodiments, a sanitizing layer 110 may include one or more portions that release one or more sanitizing agents 120 at a first time point and one or more portions that release one or more sanitizing agents 120 at one or more time points that are different from the first time point. Accordingly, in some embodiments, sanitizing layers 110 may include portions from which the same or different sanitizing agents 120 are released, one or more portions from which sanitizing agents 120 are released at different times, one or more portions from which one or more sanitizing agents 120 are released with different intensities, and substantially any combination thereof.

Numerous substances may be used alone or in combination with other substances to prepare sanitizing layers 110. Examples of such substances include, but are not limited to, polymeric substances, sintered polymers, metals, and ceramics; non-wovens, such as Tyvex® (high density polyethylene); microporous membranes; track etched membranes; dense film structures such as polyesters, thermoplastic elastomers, and low density polyolefins, and the like.

Examples of such polymeric substances include, but are not limited to, latex rubber, polyisoprene, neoprene rubber, polybutadiene, and silicone rubber. Examples of monomers which may be used include, but are not limited to, hydroxy alkyl esters of alpha, beta-unsaturated carboxylic acids (i.e., 2-hydroxy ethylacrylate, 2-hydroxy methacrylate, hydroxypropylacrylate, methacrylate, and the like). Many derivatives of acrylic or methacrylic acid may be used to form polymers. Examples of these include, but are not limited to, dimethylaminoethyl methacrylate, piperidinoethyl methacrylate, morpholinoethyl methacrylate, methacrylylglycolic acid, methacrylic acid, the monomethacrylates of glycol, glycerol, monomethacrylates of dialkylene glycols, polyalkylene glycols, and the like. In some embodiments, acrylates may be substituted for the corresponding methacrylates. Additional examples of monomers which may be used include, but are not limited to, 2-hydroxyethyl acrylate, 2-hydroxyethyl methacrylate, diethylene glycol acrylate, diethylene glycol methacrylate, 2-hydroxypropyl acrylate, 2-hydroxypropyl methacrylate, 3-hydroxypropyl acrylate, 3-hydroxypropyl methacrylate, tetraethyleneglycol acrylate, tetraethyleneglycol methacrylate, pentaethyleneglycol acrylate, pentaethyleneglycol methacrylate, dipropyleneglycol acrylate, dipropyleneglycol methacrylate, acrylamide, methacrylamide, diacetone acrylamide, methylolacrylamide, methylolmethacrylanide, acrylic acid, methacrylic acid, itaconic acid, aconitic acid, cinnamic acid, crotonic acid, mesaconic acid, maleic acid, fumaric acid, mono-2-hydroxypropyl aconitate, mono-2-hydroxyethyl maleate, mono-2-hydroxypropyl fumarate, mono-ethyl itaconate, monomethyl cellosolve ester of itaconic acid, monomethyl cellosolve ester of maleic acid, diethylaminoethyl acrylate, diethylaminoethyl methacrylate, dimethylaminoethyl acrylate, dimethylaminoethyl methacrylate, monoethylaminoethyl acrylate, monoethylaminoethyl methacrylate, tert-butylaminoethyl methacrylate, para-amino styrene, ortho-amino styrene, 2-amino-4-vinyl toluene, piperidinoethyl methacrylate, morpholinoethyl methacrylate, 2-vinyl pyridine, 3-vinyl pyridine, 4-vinyl pyridine, 2-ethyl-5-vinyl pyridine, dimethylaminopropyl acrylate, dimethylaminopropyl methacrylate, dimethylaminoethyl vinyl ether, dimethylaminoethyl vinyl sulfide, diethylaminoethyl vinyl ether, aminoethyl vinyl ether, 2-pyrrolidinoethyl methacrylate, 3-dimethylaminoethyl-2-hydroxy-propyl acrylate, 3-dimethylaminoethyl-2-hydroxy-propyl methacrylate, 2-aminoethyl acrylate, 2-aminoethyl methacrylate, isopropyl methacrylamide, N-methyl acrylamide, N-methyl methacrylamide, 2-hydroxyethyl acrylamide, 2-hydroxyethyl methacrylamide, 1-methacryloyl-2-hydroxy-3-trimethyl ammonium chloride, 1-methacryloyl-2-hydroxy-3-trimethyl ammonium sulfomethylate, 2-(1-aziridinyl)-ethyl methacrylate, styrene, vinyl acetate, vinyl chloride, vinylidene chloride, alkyl acrylates, alkyl methacrylates, alkoxyalkyl acrylates, alkoxyalkyl methacrylates, halogenalkyl acrylates, halogenalkyl methacrylates, cyano acrylates, cyano methacrylates, acrylonitrile, vinylbenzoate, and the like.

Polymers having a very broad range of physical and chemical properties may be obtained through selection of starting monomers and the concentration of the monomer in the reaction mixture. These properties may be further varied by altering the ratio between the monofunctional and polyfunctional monomers particularly as to the solubility and swelling capacity of the resultant polymeric substances. For example, when the polymerization reaction is carried out in the presence of a radical forming catalyst utilizing the bulk polymerization technique, hard brittle polymers may be formed.

In some embodiments, one or more sanitizing layers 110 may include polymers that include a sanitizing agent 120. Examples of such polymers include, but are not limited to, latexes that include α,α′-azobis(chloroformamadine), polymeric vinyl halides which include copper 8-quinolinolate, acrylate or methacrylate polymers that include antimicrobial agents, polymers that include phenolic compounds, stabilized hydrophilic polymers that include a disinfectant, polymeric substances that include quaternary ammonium compounds, polyvinylpyrrolidone complexed with iodine, and polyurethane complexed with iodine. Methods to make such polymers are known (i.e., U.S. Pat. Nos.: 3,325,436; 2,689,837; 2,875,097; 2,873,263; 3,966,902; 5,142,010; 5,326,841; 5,733,270; 4,381,380; herein incorporated by reference).

In some embodiments, one or more sanitizing layers 110 may include capsules or microspheres that include a sanitizing agent 120 that is released during use (i.e., U.S. Pat. Nos. 5,061,106; 5,138,719; herein incorporated by reference). Such capsules or microspheres may encapsulate one or more sanitizing agents 120 until the capsules or microspheres are deformed or broken to release the one or more sanitizing agents 120. For example, in some embodiments, a hydrogen peroxide producing compound may be integrally associated with one or more sanitizing layers 110 with a catalyst that is encapsulated within a breakable capsule that releases the catalyst upon breakage of the capsule. In some embodiments, a hydrogen peroxide producing compound and a catalyst that is encapsulated within a breakable capsule may be positioned between an impermeable layer and a vapor permeable layer 202 such that breakage of the capsule provides for the release of hydrogen peroxide vapor that can permeate the vapor permeable layer 202 and sanitize the surface of the material 100.

Substances may be formed into numerous articles through use of methods known in the art. For example, in some embodiments, substances may be formed into numerous types of articles through use of methods used to produce welded polyurethane films (U.S. Pat. No. 5,644,798; herein incorporated by reference). In other embodiments, the substances may be shaped through coating a support structure with a bond-preventing agent to attain a particular shape and subsequently coating the shaped structure of the bond-preventing agent with a polymeric bonding composition. Such methods are known (i.e., U.S. Pat. No. 5,501,669; herein incorporated by reference). Numerous additional methods may be used to produce various articles (i.e., U.S. Pat. Nos.: 5,549,924; 5,965,276; 6,370,694; 6,560,782; 6,913,758; 4,935,260; herein incorporated by reference).

The material 100 may also include one or more sanitizing agents that are integrally associated with the one or more sanitizing layers and that are substantially freely releasable from the one or more substantially continually sanitizing surfaces.

Numerous types of sanitizing agents 120 may be utilized. Sanitizing agents 120 may include, but are not limited to, antimicrobial agents, antibiotics, antiseptics, antiviral agents, viricidal agents, bactericidal agents, antifungal agents, antiparasitic agents, and the like. In some embodiments, the terms antiseptic, disinfectant, and germicide connote a sanitizing agent 120 which can kill microbes or pathogens upon contact. In some embodiments, the terms antiseptic, disinfectant, and germicide connote a sanitizing agent 120 which can inactivate a microbe or pathogen upon contact.

Sanitizing agents 120 may exist in numerous physical forms. For example, a sanitizing agent 120 may be a solid, liquid, gas, gel, and the like. In some embodiments, a sanitizing agent 120 may change physical form. For example, in some embodiments, a sanitizing agent 120 may initially be in liquid form and then vaporize. In some embodiments, a sanitizing agent 120 may initially be in solid form that may be mixed with a liquid to form a solution, emulsion, gel, and the like. In some embodiments, a sanitizing agent 120 may exist as a solid that will melt. In some embodiments, a solid sanitizing agent 120 may melt at human body temperature. In some embodiments, a sanitizing agent 120 may initially be in a solid form that sublimates.

In some embodiments, the sanitizing activity of a sanitizing agent 120 may be activated, deactivated, increased, decreased, and the like. For example, in some embodiments, a sanitizing agent 120 may be initially present in an inactive state that is activated upon contact with a fluid. In some embodiments, a sanitizing agent 120 may be initially present in an inactive state that becomes active upon contact with the atmosphere. In some embodiments, a sanitizing agent 120 may be initially present in an inactive state that becomes active upon contact with a catalyst. Accordingly, in some embodiments, the activity of a sanitizing agent 120 may be controlled through regulation of conditions associated with the sanitizing agent 120, such as concentration of sanitizing agent 120, temperature, catalyst concentration, and the like. In some embodiments, the activity of a sanitizing agent 120 may be decreased or deactivated. For example, in some embodiments, a quenching agent may be used to reduce or stop a chemical reaction used to generate a sanitizing agent 120.

Examples of sanitizing agents 120 include, but are not limited to, chlorhexidine gluconate, chlorhexidine acetate, chlorhexidine hydrochloride, chlorhexidine, other chlorhexidine salts, other hexamethylenebis biguanides, octoxynol, nonoxynol-9, methanol, ethanol, isopropanol, allyl alcohol, rubbing alcohol NF, sodium hypochlorite, potassium hypochlorite, calcium hypochlorite, magnesium hypochlorite, sodium dichloroisocyanurate, sodium perborate NF, sodium hydroxide, potassium hydroxide, magnesium hydroxide, calcium hydroxide, ammonia, ammonium hydroxide, lithium hydroxide, barium hydroxide, silver hydroxide, other metal hydroxides, sodium tetradecyl sulfate, sulfur dioxide, pentationic acid, colloidal sulfur, sulfurated potash, sublimed tyrothricin, hexachlorophene, hypochlorous acid, other chlorophors, acetic acid, hydrochloric acid, sulfuric acid, sodium acetate, aluminum acetate, acetarsone, aluminum subacetate, cadmium sulfide, selenium sulfide, other metal sulfides, bacitracin, calomel, chiniofon, creosote, diiodohydroxyquin, eucalyptol, eucalyptus oil, glycobiarsol, gramicidin, hexyl resorcinol, methylene blue, peppermint oil, phenylethyl alcohol, phenyl salicylate, methyl salicylate, pine tar, pine oil NF, pine oil emulsion, tertiary terpene alcohols, secondary terpene alcohols, alpha-terpineol, borneol, fenchyl alcohol, o-methylchavicol, polymixin B sulfate, colistin, chloramphenicol, tetracycline, erythromycin, gentamycin, mafenide acetate, neomycin sulfate, sulfisoxazole diolamine, sulfacetamide sodium, gentamycin sulfate, amphotericin B, tobramycin, a penicillin, a cephalosporin, salicylic acid, trichloroacetic acid, benzoic acid, pyrogallol NF X, pyrogallic acid, sodium benzoate, boric acid, sodium borate, lactic acid, sodium lactate, chloramine, chloramine T, silver nitrate, ammoniacal silver nitrate solution, eugenol, elemental iodine, sodium iodide, potassium iodide, calcium iodide, ammonium iodide, silver iodide, colloidal silver iodide in gelatin, silver lactate, ferrous iodide, mercuric iodide red, mercuric oxide red, strontium iodide, lithium iodide, magnesium iodide, zinc iodide, silver iodide, selenium iodide, thymol iodide NF X, dithymol diiodide, iodinated derivatives of thymol, other iodide salts, povidone-iodine, iodoform, iodinated organic compounds, iodol, iodopyrrol, other iodophors, chlorinated lime, bromide salts, sodium bromide, merbromin NF, other bromophors, other brominated chemicals, sodium fluoride and other fluorinated chemicals and fluorophors, Lysol, Nonidet P40, phenyl mercuric acetate, potassium mercuric iodide, proflavine hemisulfate, 3,6-diaminoacridine bisulfate, formaldehyde, glutaraldehyde, paraformaldehyde, butyl hydroxybenzoate, mercurous chloride, iodochlorhydroxyquin, zinc nitrate, zinc sulfate, cadmium sulfate, thimerosal NF, zinc oxide, zinc acetate, zinc chloride, silver sulfadiazine, liquid peroxide, solid hydrogen peroxide complexes, peracetic acid, hydrogen peroxide, urea hydrogen peroxide, hydrogen peroxide carbamide, benzoyl peroxide, calcium peroxide, magnesium peroxide, barium peroxide, strontium peroxide, sodium peroxide, potassium perchlorite, sodium perchlorite, calcium perchlorite, magnesium perchlorite, zinc perchlorite, zinc peroxide, zinc carbonate, zinc hydroxide, zinc sulfate, succinyl peroxide, succinchlorimide NF IX, N-Chloro-succinimide, potassium permanganate, sodium chlorate, potassium chlorate, phenol, sodium phenolate, domiphen bromide, salicylic acid, bismuth-formic-iodide, bismuth subgallate, bacitracin zinc, sodium lauryl sulfate, carbamide peroxide, sodium borate, oleic acid-iodine, piperonyl butoxide, sodium peroxyborate monohydrate, ammonium ichthosulfonate, eucalyptol, menthol, Witch Hazel, camphor, tannic acid, camphorated phenol, phenol glycerin, chloroxylenol, 4-chloro-3,5-xylenol, chloroquinaldol, nalidixic acid, zinc phenol-sulfonate, zinc sulfocarbolate, hydroxynalidixic acid, pipemidic acid, norfloxacin, norfloxacin hydrochloride, other quinolones, 8-hydroxyquinoline sulfate, sodium phenolate, thyme oil, o-cresol, m-cresol, metacresylacetate, p-cresol, cresol NF, 4-chloro-m-cresol, 4-chloro-S,5-xylenol, saponified cresol solution NF, methylphenol, ethyl phenol, other alkyl phenols, o-phenyl phenol, other aryl phenols, bis-phenols, phenyl-mecuric chloride, phenylmecuric borate, resorcinol, resorcinol monoactetate NF, orthophenylphenol, chloroxylenol, hexyl-resorcinol, parachlorophenol, paratertiary-amylphenol, thymol, chlorothymol NF, menthol, butylparaban, ethylparaben, methylparaben, propylparaben, triclosan, bithionol NF, o-benzyl-p-chlorophenol, hexachlorophene, poloxamer 188, benzalkonium chloride where the alkyl groups attached to the nitrogen represent any alkyl from CH₃ to C₁₈H₃₇, methylbenzethonium chloride, cetrimonium bromide, abikoviromycin, acetylenedicarboxamide, acetyl sulfamethoxypyrazine, triclobisonium chloride, undecoylium chlorideiodine, coal tar solution, furazolidone, nifuroxime, nitrofurazone NF, nitromersol NF, oxychlorosene, sodium oxychlorosene, parachlorophenol NF, camphorated parachlorophenol NF, phenylmercuric nitrate NF, gentian violet USP, hexamethylpara-rosaniline chloride, rosaniline chloride, pentamethylpararosaniline chloride, methylrosaniline chloride, tetramethylpararosaniline chloride, nonylphenoxypolyethoxyethanol, methoxypolyoxyetheneglycol 550 laurate, oxyquinoline benzoate, p-triisopropylphenoxypolyethoxy-ethanol, halazone NF, dichloramine-T, benzethonium chloride, econazole, cetylpyridinium chloride, methylbenzethonium chloride, cetyldimethylbenzylammonium chloride, dichlorobenzalkonium chloride, domiphen bromide, triclocarban, clotrimazole, ciclopirox olamine, undecylenic acid, miconazole, tolnaftate, acriflavine, euflavine, 3,6-diamino-10-methylacridium chloride, 3,6-diamino-acridine, acid acriflavine, 5-aminoacridine hydrochloride monohydrate, malachite green G, dodecyltrimethylammonium bromide, tetradecyltrimethylammonium bromide, dequalinium chloride BP, dibromopropamidine isethionite, hexadecyltrimethylammmonium bromide, chloroazodin NF X, N-chloro-p-toluenesulfonamidosodium, 4-[(dichloroamino)sulfonyl]-benzoic acid, methenamine, methenamine mandelate, methenamine hippurate, octoxynol 9, phenazopyridine hydrochloride, 9-aminoacridine hydrochloride, bismuth tribromophenate, p-tert-butylphenol, cetyldimethylethylammonium bromide, chlorothymol, cloflucaban, clorophene, cloroxine, 8-hydroxyquinoline, merbromin, mercuric oxide yellow, ammoniated mercury, p-tert-pentylphenol, phenylmercuric acetate, phenylmercuric nitrate, propylene oxide, zinc pyrithione, zinc bacitracin, chlortetracycline hydrochloride, calcium chlortetracycline, oxytetracycline hydrochloride, beta-propiolactone, acyclovir, acyclovir sodium, amantadine hydrochloride, cytarabine, idoxuridine, interferon, gamma interferon, ribaviron, rifampin, suramin, trifluridine, vidarabine, zidovudine, methisazone, tumor necrosis factor, ampligen, ansamycin, (E)-5-(2-bromovinyl-2′-deoxyuridine, butylated hydroxytoluene, castamospermine, dextran sulfate, dideoxycytidine, dideoxyadenosine, dideoxyinosine, Peptide-T, dihydromethylpyridinylcarbonyloxyazidodideoxythymidine, ganciclovir, 2′-fluoro-2′-deoxy-5-iodo-ara C, phosphonoformate, rimantadine hydrochloride, and their derivatives and mixtures thereof. An example of a sanitizing agent 120 that is a multicomponent mixture includes ethanol and two organic acids. In some embodiments, such a mixture includes 2% malic acid and 2% citric acid in a standard ethanol hand-sanitizing solution. This mixture has been shown to effectively inactivate rhinovirus (Smith, Interscience Conference on Antimicrobial Agents and Chemotherapy (ICAAC): Rhinovirus on Hands Blocked By Solution for Hours, Conference Report, Oct. 2, 2006, On-line report, Medpage Today, LLC, Little Falls, N.J.). Accordingly, numerous sanitizing agents 120 are known in the art and have been reported (i.e., U.S. Pat. Nos.: 5,667,753; 6,193,931; 5,357,636; herein incorporated by reference).

The material 100 may optionally include one or more reservoirs for the one or more sanitizing agents 130. In some embodiments, the one or more reservoirs 130 may be integrally associated with a sanitizing layer 110. For example, in some embodiments, one or more reservoirs 130 may be contained within a sanitizing layer 110. In some embodiments, such reservoirs 130 may be continuously connected to one or more sanitizing surfaces 112 such that one or more sanitizing agents 120 may travel directly from a reservoir 130 to the sanitizing surface 112. In some embodiments, one or more reservoirs 130 may be remotely connected to one or more sanitizing surfaces 112 such that one or more sanitizing agents 120 may travel from a reservoir 130 through a connector, such as tubing, to the sanitizing surface 112. In some embodiments, a reservoir 130 may include a device that can propel one or more sanitizing agents 120 from a reservoir 130 to a sanitizing surface 112. In some embodiments, such a device may be a pump. In some embodiments, such a device may be a thermally activated compression device. For example, a compression device may include a hydrocarbon having a high vapor pressure at body temperature that is contained within a tube having a plunger such that heating the hydrocarbon to body temperature causes the plunger to move and propel a sanitizing agent 120. In some embodiments, devices that propel sanitizing agents 120 may be associated with, or included within, one or more operating units 140.

The one or more reservoirs 130 may optionally include one or more operating units associated with the one or more reservoirs for the one or more sanitizing agents. In some embodiments, one or more operating units 140 may be associated with one or more reservoirs 130. In some embodiments, an operating unit 140 may propel one or more sanitizing agents 120 from a reservoir 130 to a sanitizing surface 112. In some embodiments, an operating unit 140 may act to control the action of a device that propels one or more sanitizing agents 120 from a reservoir 130 to a sanitizing surface 112.

In some embodiments, the one or more operating units provide one or more user interfaces. In some embodiments, one or more users may interact with one or more operating units 140 through one or more user interfaces 150. Such user interaction can include, but is not limited to, controlling an operating unit 140 with regard to parameters associated with the operating unit 140, a sanitizing layer 110, and/or a sanitizing agent 120. Parameters may include, but are not limited to, the time, place, duration, intensity, priority, and/or identity of one or more sanitizing agents 120 that are used to sanitize a sanitizing layer 110.

User interaction may occur directly or indirectly. For example, in some embodiments, a user may directly interact with an operating unit 140 through one or more user interfaces 150 that include, but are not limited to, switches, levers, buttons, a keyboard, a touchpad, and the like. In some embodiments, a user may interact with an operating unit 140 indirectly by transmitting one or more signals from one or more user interfaces 150 that are received by an operating unit 140 that control transport of a sanitizing agent 120 to a sanitizing layer 110. In some embodiments, a user is human. In some embodiments, a user is not human.

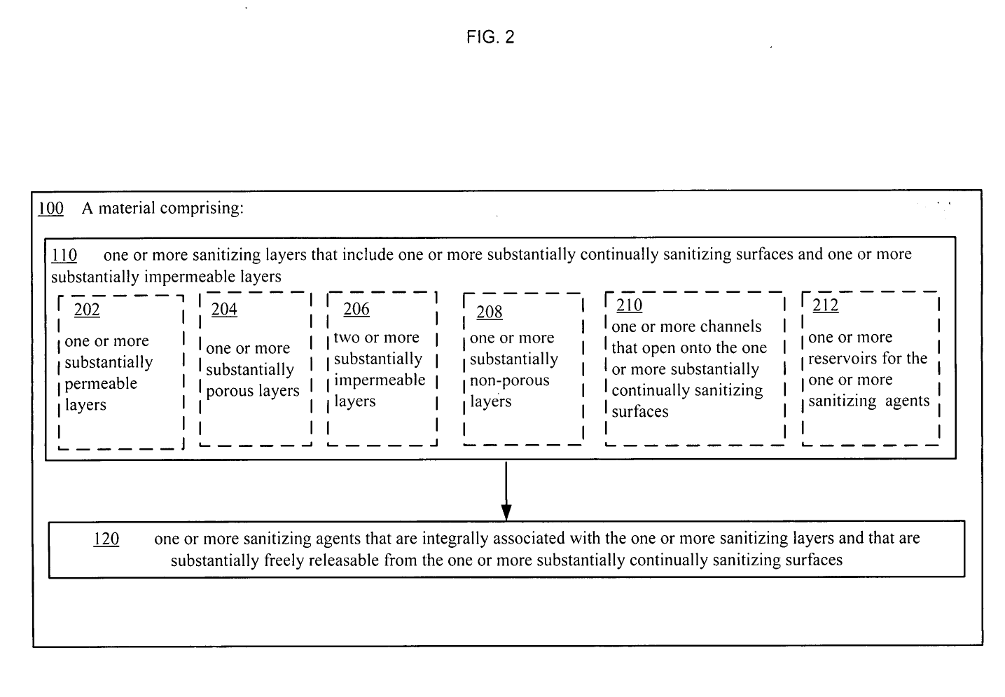

FIG. 2 illustrates alternative embodiments of the material 100 of FIG. 1. FIG. 2 illustrates example embodiments of the one or more sanitizing layers that include one or more substantially continually sanitizing surfaces and one or more substantially impermeable layers 110. Additional embodiments may include embodiment 202, embodiment 204, embodiment 206, embodiment 208, embodiment 210, and/or embodiment 212.

At embodiment 202, the one or more sanitizing layers that include one or more substantially continually sanitizing surfaces and one or more substantially impermeable layers may include one or more substantially permeable layers. In some embodiments, a substantially permeable layer 202 may include channel networks that pass through a layer. In some embodiments, the channel networks are arranged in a discontinuous manner. In some embodiments, the channel networks are arranged in a continuous manner. For example, in some embodiments, a substantially permeable layer 202 is loosely woven to allow passage of molecules through the layer. In some embodiments, one or more sanitizing layers 110 may include one or more substantially permeable layers 202 that are permeable in a phase dependent manner. For example, in some embodiments, the one or more substantially permeable layers 202 may be permeable to liquids. For example, in some embodiments, the one or more substantially permeable layers 202 may be permeable to gases. In some embodiments, the one or more substantially permeable layers 202 may be permeable to gases and vapors while being impermeable to liquids. In some embodiments, the one or more substantially permeable layers 202 may be permeable to gases and impermeable to solids. In some embodiments, the one or more substantially permeable layers 202 may be permeable to gases and liquids and impermeable to solids. In some embodiments, the one or more substantially permeable layers 202 may be vapor permeable and impermeable to air and moisture. In some embodiments, the one or more substantially permeable layers 202 may be permeable in a molecular weight dependent manner. For example, in some embodiments, the one or more substantially permeable layers 202 may be permeable to molecules of low molecular weight and impermeable to molecules of high molecular weight. In some embodiments, the one or more substantially permeable layers 202 may be permeable to molecules in a charge dependent manner. For example, in some embodiments, the one or more substantially permeable layers 202 may be permeable to charged molecules but impermeable to uncharged molecules. In some embodiments, the one or more substantially permeable layers 202 may be permeable to uncharged molecules but impermeable to charged molecules. In some embodiments, the one or more substantially permeable layers 202 may be permeable to molecules in a polarity dependent manner. For example, in some embodiments, the one or more substantially permeable layers 202 may be permeable to polar molecules but impermeable to non-polar molecules. In some embodiments, the one or more substantially permeable layers 202 may be permeable to non-polar molecules but impermeable to polar molecules.

Numerous substances may be used to construct one or more sanitizing layers 110. Substances that may be used to construct substantially permeable layers 202 can be prepared through use of numerous methods. For example, methods to prepare substances that are permeable to gas and vapor while being impermeable to fluids are known (i.e., U.S. Pat. Nos.: 4,194,041; 4,443,511; 4,692,369; 4,925,732; 5,102,711; 5,948,707; 6,521,552; 5,783,290; all of which are hereby incorporated by reference). Methods to prepare substances that are vapor permeable and impermeable to air and moisture are known (i.e., U.S. Pat. No.: 6,901,712; hereby incorporated by reference). Methods to prepare substances that are selectively gas permeable are known (i.e., U.S. Pat. No.: 6,663,805; hereby incorporated by reference). In some embodiments, apertured thermoplastic films may be used to construct permeable layers 202. In some embodiments, apertured thermoplastic films that are treated with a surfactant may be used to construct permeable layers 202. Such apertured thermoplastic films are known (i.e., U.S. Patent Statutory Invention Registration No. H1,670; herein incorporated by reference). Methods to prepare liquid permeable substances are known (i.e., U.S. Pat. No.: 5,851,551; herein incorporated by reference). In some embodiments, a substantially permeable layer 202 may include one or more apertured polymeric film webs that are liquid permeable (i.e., U.S. Published Patent Application No. 20050214506; herein incorporated by reference). Methods to prepare substances that are selectively permeable based on molecular weight are known (i.e., U.S. Pat. No.: 5,428,123; herein incorporated by reference).

Accordingly, sanitizing layers 110 may be designed that transport one or more sanitizing agents 120 to a substantially continually sanitizing surface 112 of the sanitizing layer 110 through capillary action. Methods to prepare layers that can transport fluids through capillary action are known (i.e., U.S. Published Patent Application No. 20050214506; herein incorporated by reference). In some embodiments, such methods may be used to prepare fluid permeable webs that may transport one or more sanitizing agents 120 through capillary action.

In some embodiments, sanitizing layers 110 may include two or more layers that each have physical properties that are different from each other. For example, in some embodiments, a hydrophobic layer and a hydrophilic layer may be oriented relative to each other to promote and/or inhibit the movement of hydrophobic and/or hydrophilic substances relative to the hydrophobic and/or hydrophilic layers. Such constructions of layers may be used to promote and/or inhibit wicking of substances, such as sanitizing agents 120, through the sanitizing layer 110. In some embodiments, such constructions of layers may be used to direct the flow of substances relative to the sanitizing layer 110.

In some embodiments, permeable substances may be prepared by polymerizing monomers into polymers. Examples of monomers that may be used to prepare substantially permeable layers 202 are described herein and are known in the art.

In some embodiments, cross-linking agents may be used to produce polymeric substances having various degrees of permeability. Numerous cross-linking agents may be used that include, but are not limited to, diesters of acrylic acid, diesters of methacrylic acid, ethyleneglycol diacrylate, ethyleneglycol dimethacrylate, 1,2-butyleneglycol diacrylate, 1,2-butyleneglycol dimethacrylate, 1,3-butyleneglycol diacrylate, 1,3-butyleneglycol dimethacrylate, 1,4-butyleneglycol diacrylate, 1,4-butyleneglycol dimethacrylate, propyleneglycol diacrylate, propyleneglycol dimethacrylate, diethyleneglycol diacrylate, diethyleneglycol dimethacrylate, dipropyleneglycol diacrylate, dipropyleneglycol dimethacrylate, divinyl benzene, divinyl toluene, diallyl tartrate, allyl pyruvate, allyl maleate, divinyl tartrate, triallyl melamine, N,N′-methylene bis acrylamide, glycerine dimethacrylate, glycerine trimethacrylate, diallyl maleate, divinyl ether, diallyl monoethyleneglycol citrate, ethyleneglycol vinyl allyl citrate, allyl vinyl maleate, diallyl itaconate, ethyleneglycol diester of itaconic acid, divinyl sulfone, hexahydro 1,3,5-triacyltriazine, triallyl phosphite, diallyl ether of benzene phosphonic acid, maleic anhydride triethylene glycol polyester, polyallyl sucrose, polyallyl glucose, sucrose diacrylate, glucose dimethacrylate, pentaerythritol di-, tri- & -tetraacrylate or methacrylate, trimethylol propane di- and triacrylate or methacrylate, sorbitol dimethacrylate, 2-(1-aziridinyl)-ethyl methacrylate, tri-ethanolamine diacrylate or dimethacrylate, triethanolamine triacrylate or trimethacrylate, tartaric acid dimethacrylate, triethyleneglycol dimethacrylate, the dimethacrylate of bis-hydroxy ethylacetamide and the like.

If the polymerization reaction is conducted in the presence of a solvent, soluble linear or branched chain complex polymers and copolymers may be obtained. In some embodiments, conducting the polymerization reaction in the substantial absence of any solvent produces products that constitute rigid macroporous polymer substances. In some embodiments, polymerization may be carried out in the presence of a solvent which is effective for only partially swelling the polymer such that soft sponge-like polymer products are obtained. Accordingly, the degree of cross-linking which takes place in the polymerization reaction also influences the properties of the resultant polymeric products.

Examples of solvents include, but are not limited to, hydrophilic substances such as water, alcohol, ketone, glycol, glycol ester, glycol ether, amide, alkyl amide, and the like. In some embodiments, a hydrophilic solvent can be replaced by an appropriate hydrophobic solvent, such as an aromatic, aliphatic or halogenated hydrocarbon, ether, ester, or the like.

The polymerization reactions may be initiated in the conventional manner through use of radical forming initiators. Examples of such initiators include, but are not limited to, dibenzoyl peroxide, tert-butyl peroctoate, cumene hydroperoxide, diazodilsobutyrodinitrille, diisopropylpercarbonate, ammonium persulfate, and the like, alone or in combination with a reducing agent.

In some embodiments, one or more permeable sheets may be laminated together to form a substantially permeable layer 202 of one or more sanitizing layers 110. In some embodiments, one or more permeable sheets may be laminated together to form a selectively permeable layer. For example, in some embodiments, a sheet that is impermeable to one or more charged chemical compounds may be laminated onto a sheet that is impermeable to one or more chemical compounds that are above a given mass (i.e., 150 grams/mole). Accordingly, such a semi-permeable layer will be permeable to uncharged chemical compounds having a molecular mass that is below the given mass (i.e., 150 grams/mole). Numerous combinations may be used to control the permeability of one or more sanitizing layers 110. Methods to prepare semi-permeable layers are known and have been described (i.e., U.S. Pat. No.: 5,648,003; herein incorporated by reference).

In some embodiments, one or more sanitizing layers 110 may include one or more substantially permeable layers 202 that can control the release of one or more sanitizing agents 120 from the one or more sanitizing layers 110. In some embodiments, one or more sanitizing layers 110 may include one or more substantially permeable layers 202 that can control the release of one or more sanitizing agents 120 from one or more substantially continually sanitizing surfaces 112 of the one or more sanitizing layers 110.

In some embodiments, one or more sanitizing layers 110 may include one or more substantially permeable layers 202 that provide for the release of one or more sanitizing agents 120 in vapor and/or gas form from one or more sanitizing layers 110. In some embodiments, one or more sanitizing layers 110 may include one or more substantially permeable layers 202 that may control the release of one or more sanitizing agents 120 in vapor and/or gas form from one or more substantially continually sanitizing surfaces 112. For example, in some embodiments, one or more substantially permeable layers 202 may provide for release of peroxide vapor. In some embodiments, the peroxide vapor may be released from one or more surfaces of the one or more sanitizing layers 110. In some embodiments, the peroxide vapor may be released from one or more substantially continually sanitizing surfaces 112 of the one or more sanitizing layers 110. In some embodiments, the peroxide vapor may be generated through use of one or more inorganic hydrogen peroxide complexes. Numerous inorganic hydrogen peroxide complexes exist and include, but are not limited to, alkalimetal carbonates, ammonium carbonates, alkali metal oxalates, alkali metal phosphates, alkali metal pyrophosphates fluorides, hydroxides, sodium carbonate hydrogen peroxide complexes, complexes of hydrogen peroxide with polymeric N-vinylheterocyclic compounds, complexes of hydrogen peroxide and solid polymeric electrolytics. Such complexes are known and have been described (i.e., U.S. Pat. Nos.: 2,986,448; 3,870,783; 3,376,110; 3,480,557; 5,008,093; 5,077,047; 5,030,380; herein incorporated by reference).

In some embodiments, one or more sanitizing layers 110 may include one or more permeable layers 202 that provide for the release of one or more sanitizing agents 120 in liquid and/or gel form from one or more sanitizing layers 110. In some embodiments, one or more sanitizing layers 110 may include one or more permeable layers 202 that may control the release of one or more sanitizing agents 120 in liquid and/or gel form from one or more substantially continually sanitizing surfaces 112. Numerous sanitizing agents 120 may be prepared as liquids and/or gels for release from one or more sanitizing layers 110. Examples of such sanitizing agents 120 that may be prepared as liquids and/or gels include, but are not limited to, alkalies/hydroxides (i.e., sodium hydroxide, caustic soda, soda lye, calcium oxide (lime)), biguanides/chlorhexidine (i.e., volvasan®, virosan, chlorhexiderm), cationic surfactants/quaternary ammonium compounds (i.e., parvosol™, roccal-D® plus, A33™, maxima 128, ken-care, unicide 256, benzalkonium chloride, bensathonium chloride, cetylpyridinium chloride), halogens and halogen-containing compounds (i.e., sodium hypochlorite (chlorine bleach), alcide, sodium dichloroisocyanurate, calcium hypochlorite), iodine-based (iodine, iodophors, povidone-iodine, betadine), oxidizing agents/peroxides (i.e., ozone, hydrogen peroxide, sodium perborate, benzoyl peroxide, potassium permanganate), peroxygen compounds (i.e., stabilized chlorine dioxide), phenols and related compounds/phenolics (i.e., phenol (carbolic acid), cresol (cresylic acid), lysol, pine tar, pine oil), synthetic phenol (i.e., chloroxylenols, hexachlorophene, sporicidin, parachlorometaxylenol (PCMX), dichlorometaxylenol (DCMX)), reducing agents/aldehydes (i.e., glutaral (glutaraldehyde), formalin (formaldehyde)), and alcohols. Such sanitizing agents 120 are known (i.e., U.S. Pat. Nos.: 4,642,165; 4,744,951; 5,008,106; herein incorporated by reference).

In some embodiments, the permeability of a substantially permeable layer 202 may be dependent upon stretching or deformation of the substantially permeable layer 202. For example, in some embodiments, the permeability of a substantially permeable layer 202 in a compressed form may be low but may be high when the same layer is stretched to deform the layer and thereby increase the permeability of the layer.

At embodiment 204, the one or more sanitizing layers that include one or more substantially continually sanitizing surfaces and one or more substantially impermeable layers may include one or more substantially porous layers. In some embodiments, a substantially porous layer 204 includes passages that pass through a layer in a continuous manner.

Numerous types of substances may be used to prepare sanitizing layers 110 that include one or more substantially porous layers 204. For example, porous plastics may be used to prepare such substantially porous layers 204. Examples of such porous plastics include, but are not limited to, thermoplastic polymers, such as polyethylene, polypropylene, polyvinylferrocene, nylon, polyether sulphone, expanded polytetrafluoroethylene films, and the like. Substantially porous layers 204 may be prepared through use of methods as described herein and as are known in the art (i.e., U.S. Pat. Nos.: 6,676,871; 3,953,566; 6,252,128; 4,187,390; 6,765,029; 5,798,165; 5,779,795; and 5,641,566; herein incorporated by reference). In some embodiments, a substantially porous layer 204 may be metallic (i.e., U.S. Pat. No. 5,641,566; herein incorporated by reference).

In some embodiments, a substantially porous layer 204 may be prepared from one or more sheets such as those sold by Porex Technologies (Fairburn, Ga., Porex®). Porex® is microporous with known pore size. Porex® is permeable to vapor, but not aqueous liquids (i.e., it is hydrophobic). In some embodiments, the Porex® may be used to provide for constant delivery and release of a sanitizing agent 120 from the surface of a sanitizing layer 110 (i.e., U.S. Pat. No. 5,733,270; herein incorporated by reference). Additionally, Porex® is thought to be impervious to microorganisms.

In some embodiments, pores may be created in a substantially porous layer 204 through physical methods (i.e., U.S. Pat. No. 5,269,981; herein incorporated by reference). Briefly, a sheet may be placed on a pattern anvil having a pattern of raised areas wherein the height of the raised areas is greater than the thickness of the sheet; the sheet is conveyed, while placed on the pattern anvil, through an area where a fluid is applied to the sheet; and the sheet is subjected to a sufficient amount of ultrasonic vibration in the area where the fluid is applied to the sheet to microaperture the sheet in a pattern generally the same as the pattern of raised areas on the pattern anvil.

In some embodiments, the density and structure of the pores associated with a sanitizing layer 110 control, at least in part, delivery of a sanitizing agent 120 to the surface of a sanitizing surface 112 and release of the sanitizing agent 120 from the surface of a sanitizing layer 110. Through selection of pore structure, size of the area for permeation, control of length and height, and selection of material, the delivery of a sanitizing agent 120 and release of the sanitizing agent 120 from the surface of a sanitizing surface 112 may be controlled.

At embodiment 206, the one or more sanitizing layers that include one or more substantially continually sanitizing surfaces and one or more substantially impermeable layers may include two or more substantially impermeable layers.

Substantially impermeable layers 114 can include layers that are impermeable to liquids, gases, and solids. Substantially impermeable layers 114 may have numerous configurations and be made in numerous ways.

In some embodiments, substantially impermeable layers 114 may include water-impermeable and gas-impermeable sheets. Methods to prepare water-impermeable sheets and essentially gas-impermeable thermoplastic elastomers are known and have been described (U.S. Pat. Nos.: 4,312,907 and 7,056,971; herein incorporated by reference).

In some embodiments, the substantially impermeable layer 114 may include a thermoplastic polymer. An example of such a thermoplastic polymer includes, but is not limited to, high-density polyethylene. In some embodiments, at least some of the high-density polyethylene may be replaced by other polymers that may include, but are not limited to, polypropylene and its copolymers, such as polypropylene/polyethylene, and terpolymers, such as poly-(propylene-butene/ethylene). In some embodiments, poly-(ethylene-vinylalcohol) may be mixed with high-density polyethylene (see U.S. Pat. No. 5,731,053; herein incorporated by reference). In some embodiments, a substantially impermeable layer 114 may include one or more styrene-ethylene-butylene-styrene copolymers, one or more styrene-butadiene-styrene copolymers, and/or one or more styrene-isoprene-styrene copolymers (see U.S. Pat. No. 5,480,915; herein incorporated by reference).

In some embodiments, one or more impermeable layers 114 may include one or more elastic sheets that include a substantially continuous ductile metal coating that is able to repeatedly expand and contract with the elastic sheet. Such metal coated elastic sheets are able to expand and contract without fracturing or breaking the metal layer. Methods to prepare such metal coated elastic sheets are known (i.e., U.S. Pat. Nos.: 5,069,227; 5,113,874; herein incorporated by reference).

At embodiment 208, the one or more sanitizing layers that include one or more substantially continually sanitizing surfaces and one or more substantially impermeable layers may include one or more substantially non-porous layers.

In some embodiments, non-porous layers 208 may include modified polysiloxane based elastomers that include polydimethylsiloxane based elastomers, ethylene-propylene diene based elastomers, polynorbornene based elastomers, polyoctenamer based elastomers, polyurethane based elastomers, butadiene and nitrile butadiene rubber based elastomers, natural rubber, butyl rubber based elastomers, polychloroprene based elastomers, epichlorohydrin elastomers, polyacrylate elastomers, polyethylene, polypropylene, polytetrafluoroethylene, polyvinylidene difluoride based elastomers, and mixtures thereof (i.e., U.S. Pat. No.: 6,716,352; herein incorporated by reference). For example, in some embodiments, a non-porous layer 208 may be prepared from a non-porous polyurethane film block copolymer. Such polyurethane film block copolymers may be prepared through reaction of a diisocyanate and a polyethylene glycol that is present in the amount of from 25 to 45% by weight based on the total weight of the reaction mixture, to produce the non-porous polyurethane film block copolymer.

In some embodiments, a substantially non-porous layer 208 may include a fluid impermeable foil. Such foils are known and may be prepared by vapor depositing a metallic film onto a substrate (i.e., U.S. Pat. No. 6,524,698; herein incorporated by reference). Numerous methods that may be used to prepare non-porous layers are known and have been described.

At embodiment 210, the one or more sanitizing layers that include one or more substantially continually sanitizing surfaces and one or more substantially impermeable layers may include one or more channels that open onto the one or more substantially continually sanitizing surfaces.

In some embodiments, one or more substantially continually sanitizing surfaces 112 may include one or more channels 210 on their surfaces. In some embodiments, one or more substantially continually sanitizing surfaces 112 may include one or more channels 210 that are contained within the one or more sanitizing layers 110 and that open onto the one or more sanitizing surfaces 112. In some embodiments, one or more sanitizing agents 120 may travel through the one or more channels 210 through capillary action. In some embodiments, the one or more channels 210 may be continuously connected to one or more reservoirs 130 that may contain one or more sanitizing agents 120.

In some embodiments, one or more sanitizing agents 120 may be propelled through one or more channels 210 through the use of a propellant. In some embodiments, one or more sanitizing agents 120 may be contained within a reservoir 130 that is connected to the one or more channels 210 such that a sanitizing agent 120 may be propelled from the reservoir 130 through the channels 210 onto a sanitizing surface 112 through use of a propellant. Numerous compounds may be used as propellants. For example, in some embodiments, low molecular weight hydrocarbons, such as alkanes, alkynes, alkenes, alcohols, ethers, esters, and the like, may be used as propellants. In some embodiments, such hydrocarbons may exist as liquids at room temperature that exhibit a high vapor pressure. Accordingly, such fluid hydrocarbons may be caused to vaporize and thereby propel sanitizing agents 120 through one or more channels 210. In some embodiments, hydrocarbons may be selected that vaporize at human body temperature, such as pentane, to propel a sanitizing agent 120 through one or more channels 210 upon contact of a material 100 containing the one or more channels 210 with a human. Numerous propellants may be used to propel a sanitizing agent 120 through one or more channels 210.

In some embodiments, one or more pumps may be connected to one or more channels 210 such that action of the one or more pumps will propel one or more sanitizing agents 120 through the one or more channels 210. In some embodiments, one or more of the pumps may be controlled through use of an operating unit 140.

Release of one or more sanitizing agents 120 from a channel 210 can be controlled through controlling the size of the channel 210 and the pressure exerted on the channel 210. Accordingly, channel 210 characteristics may be selected to control the release of one or more sanitizing agents 120 from a substantially continually sanitizing surface 112.

At embodiment 212, the one or more sanitizing layers that include one or more substantially continually sanitizing surfaces and one or more substantially impermeable layers may include one or more reservoirs for the one or more sanitizing agents.

In some embodiments, a sanitizing layer 110 may include one or more reservoirs 130. In some embodiments, such reservoirs 130 may contain one type of sanitizing agent 120. In some embodiments, such reservoirs 130 may contain one or more type of sanitizing agent 120. In some embodiments, such reservoirs 130 may contain components other than sanitizing agents 120. For example, such reservoirs 130 may contain coloring agents, olfactory agents, and the like.

In some embodiments, reservoirs 130 may be integrally associated with a sanitizing layer 110. For example, in some embodiments, a reservoir 130 may be contained within a sanitizing layer 110 that opens onto the surface of the sanitizing layer 110. In other embodiments, a sanitizing layer 110 may include a reservoir 130 that connects to a channel 210 that opens onto the surface of the sanitizing layer 110. Accordingly, reservoirs 130 may be configured in numerous geometries.

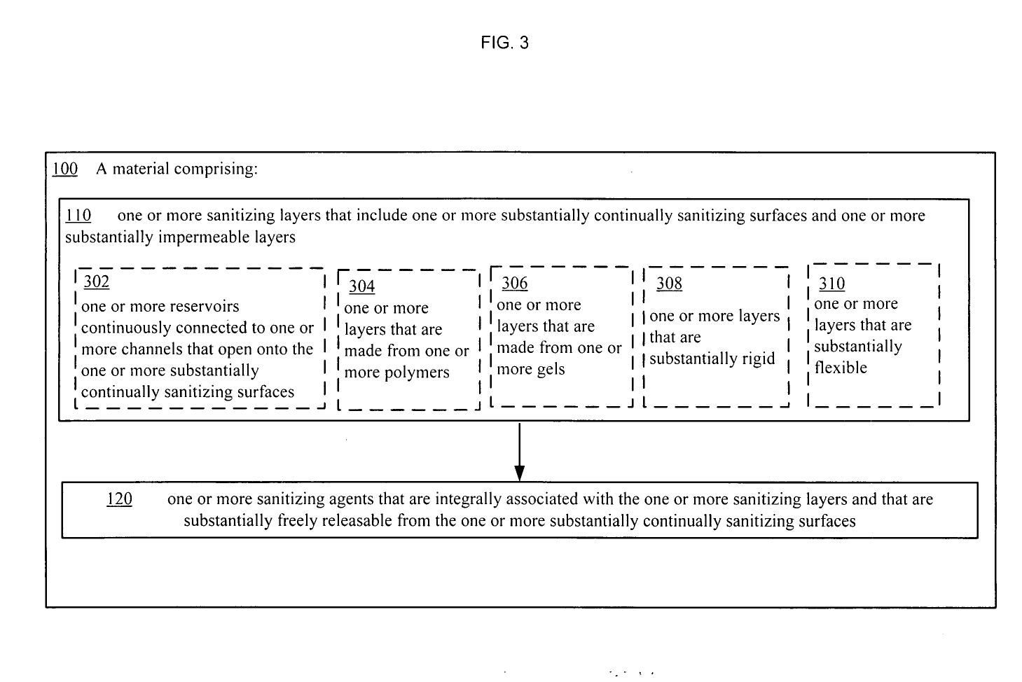

FIG. 3 illustrates alternative embodiments of the material 100 of FIG. 1. FIG. 3 illustrates example embodiments of the one or more sanitizing layers that include one or more substantially continually sanitizing surfaces and one or more substantially impermeable layers 110. Additional embodiments may include embodiment 302, embodiment 304, embodiment 306, embodiment 308, and/or embodiment 310.

At embodiment 302, the one or more sanitizing layers that include one or more substantially continually sanitizing surfaces and one or more substantially impermeable layers may include one or more reservoirs continuously connected to one or more channels that open onto the one or more substantially continually sanitizing surfaces.

Reservoirs 130 may be configured in numerous geometries. In some embodiments, the one or more reservoirs 130 may be contained within one or more sanitizing layers 110. In some embodiments, the one or more reservoirs 130 may be separate from the one or more sanitizing layers 110 and connected to the one or more sanitizing layers 110 through use of a connector, such as tubing. For example, in some embodiments, a reservoir 130 may be a tank that is connected to a sanitizing layer 110 through the use of tubing that allows a sanitizing agent 120 to be transported from the reservoir 130 to the sanitizing layer 110. In some embodiments, a pump, compressor, or other device may be associated with the reservoir 130 to propel a sanitizing agent 120 to an associated sanitizing layer 110. Such devices may be associated with an operating unit 140 to provide for controlled delivery of a sanitizing agent 120 to a sanitizing layer 110. For example, such devices may include circuitry that can be used to control operation of the device. In some embodiments, the circuitry may be used to control delivery of a sanitizing agent 120 from a reservoir 130 to a sanitizing surface 112 upon receipt of a signal.

At embodiment 304, the one or more sanitizing layers that include one or more substantially continually sanitizing surfaces and one or more substantially impermeable layers may include one or more layers that are made from one or more polymers.

Numerous polymers are known and have been described herein that may be used to prepare one or more sanitizing layers 110. Sanitizing layers 110 that include polymers may exhibit numerous properties. Examples of such properties include, but are not limited to, hardness, softness, malleability, pliability, flexibility, stiffness, permeability, impermeability, porosity, non-porosity, elasticity, chemical imperviousness, thermal conductivity, insulation ability, conductivity, hydrophobicity, hydrophilicity, electrical conductivity, self-siphoning ability, and the like.

At embodiment 306, the one or more sanitizing layers that include one or more substantially continually sanitizing surfaces and one or more substantially impermeable layers may include one or more layers that are made from one or more gels.

In some embodiments, one or more sanitizing layers 110 may include gelatinous elastomer compositions that include a selectively hydrogenated triblock copolymer of styrene and butadiene and an excess by weight of a plasticizing oil (i.e., U.S. Pat. No.: 4,618,213; herein incorporated by reference). In some embodiments, one or more sanitizing layers 110 may include a hydrocarbon oil and a mixture of two selectively hydrogenated styrene/butadiene triblock polymers of particular composition in a particular weight ratio (i.e., U.S. Pat. No.: 4,716,183; herein incorporated by reference). In some embodiments, styrene-diene block copolymers may be used. A styrene-diene block copolymer is a poly (styrene-ethylene-butylene-styrene) triblock copolymer (SEBS triblock copolymers) which, when combined with sufficient plasticizer, such as a hydrocarbon oil, provides a gel composition. For example, these triblock copolymers may be melt blended with oils to produce a gel-like polymer which is meltable and useful for cast molding of shaped articles. Numerous gels and methods to produce them are known and have been reported (i.e., U.S. Pat. Nos.: 4,942,270; 4,369,284; 4,618,213; 3,827,999; 4,176,240; 3,485,787; 3,376,384; 4,716,183; 4,556,464; 4,499,154; herein incorporated by reference).

In some embodiments, the pore size and/or the permeability of porous and/or permeable gels may be selected to control release of one or more sanitizing agents 120 from a sanitizing surface 112. For example, in some embodiments, a sanitizing layer 110 may include a gel having very large pores that provides for greater release of one or more sanitizing agents 120 relative to a gel having smaller pores. Accordingly, sanitizing agent 120 release may be regulated through control of pore size.

In some embodiments, gels may be selected in which the pore size may be dynamically controlled. For example, pores may be opened or closed following a select event or condition. For example, in some embodiments, gels may be selected in which the pore size and/or permeability is temperature dependent. In other embodiments, a gel may be selected in which the pore size and/or permeability of the gel is dependent upon electrical current flowing through the gel. Accordingly, the release of one or more sanitizing agents 120 from a sanitizing layer 110 that includes such a gel may be regulated by controlling the amount of electrical current applied to the gel. In some embodiments, gels may be selected in which the pore size and/or permeability is dependent upon exposure to one or more chemicals. Accordingly, the release of one or more sanitizing agents 120 from a sanitizing layer 110 that includes such a gel may be regulated by controlling the exposure of one or more chemicals to the gel.

At embodiment 308, the one or more sanitizing layers that include one or more substantially continually sanitizing surfaces and one or more substantially impermeable layers may include one or more layers that are substantially rigid.

Generally, layers that are substantially rigid include those that are substantially resistant to being bent, flexed, and/or stretched. However, designation of a layer as being substantially rigid does not mean that the layer is entirely unable to bend, flex, and/or stretch. For example, a substantially rigid layer may be constructed from stainless steel such that it is substantially resistant to being bent but still exhibits a moderate ability to flex. Examples of other substances that may be used to construct substantially rigid layers include, but are not limited to, wood, metal, plastic, stone, ceramic, hard rubber, tile, glass, and the like.

One or more sanitizing layers 110 that include one or more substantially rigid layers 308 may be used to construct numerous articles. In some embodiments, such layers may be used to construct tables and table tops having substantially continually sanitizing surfaces 112. For example, in some embodiments, countertops (i.e., such as those found in kitchens, hospitals, bathrooms, pharmaceutical manufacturing plants, food processing and packaging areas, and the like) may be constructed that include one or more sanitizing surfaces 112. In some embodiments, tables (i.e., such as surgical tables, hospital examination tables, food preparation tables, and the like) may include one or more sanitizing surfaces 112. In some embodiments, building features may be constructed that include one or more sanitizing surfaces 112. Such building features include, but are not limited to, poles, beams, handrails, handles (i.e., door handles and door knobs), bathroom fixtures, floor tiles, siding, ceiling tiles, chairs, and the like. In some embodiments, tools may include one or more sanitizing surfaces 112.

At embodiment 310, the one or more sanitizing layers that include one or more substantially continually sanitizing surfaces and one or more substantially impermeable layers may include one or more layers that are substantially flexible.

Generally, layers that are substantially flexible include those that can be substantially bent, flexed, and/or stretched. However, designation of a layer as being substantially flexible does not mean that the layer lacks structural rigidity. For example, a substantially flexible layer may be constructed from latex rubber such that it may be significantly bent, flexed, and/or stretched while being able to return to its initial shape. Examples of other substances that may be used to construct substantially flexible layers include, but are not limited to, metal, plastic, ceramic, rubber, glass, and the like. In some embodiments, a single type of substance may be used to construct a substantially flexible layer while in other embodiments the substance may be used to construct a substantially rigid layer. For example, in some embodiments, glass may be used to construct a substantially rigid layer while in other embodiments, glass fibers may be used to construct a substantially flexible layer.

One or more sanitizing layers 110 that include one or more substantially flexible layers may be used to construct numerous articles. In some embodiments, such layers may be used to construct gloves, fabrics, clothing, sheets, surgical gowns, surgical drapes, laboratory coats, condoms, diaphragms, seat covers, table cloths, sanitizing wipes, covers for building features (i.e., poles, benches, chairs, handles, and the like), diapers, and the like.

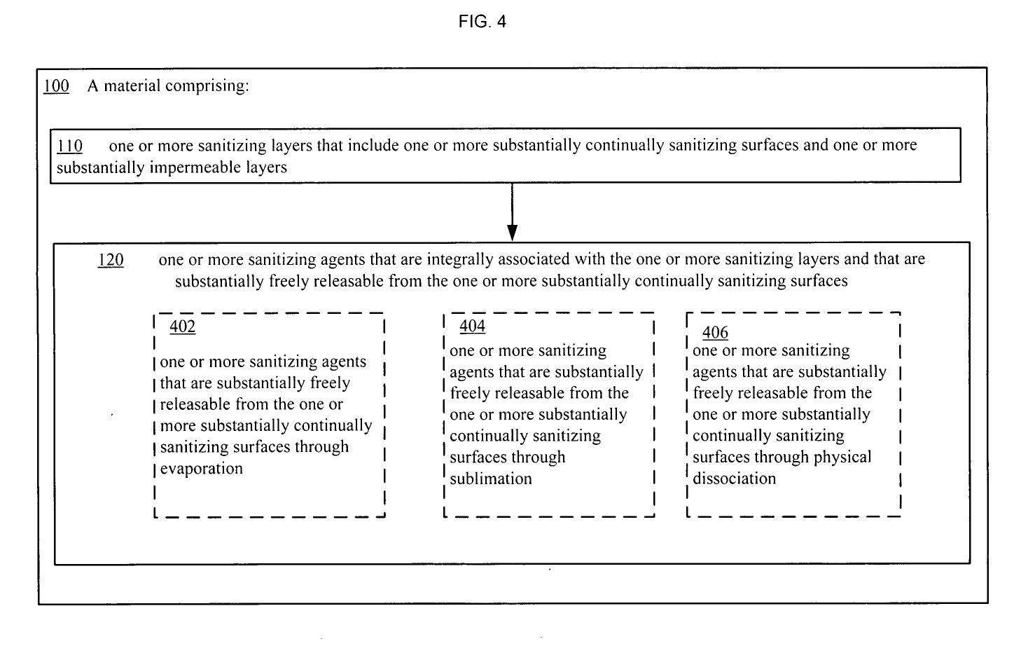

FIG. 4 illustrates alternative embodiments of the material 100 of FIG. 1. FIG. 4 illustrates example embodiments of the one or more sanitizing agents that are integrally associated with the one or more sanitizing layers and that are substantially freely releasable from the one or more substantially continually sanitizing surfaces 120. Additional embodiments may include embodiment 402, embodiment 404, and/or embodiment 406.

At embodiment 402, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through evaporation.

Sanitizing agents 120 that are able to transition from the liquid phase to the gaseous phase may be used in association with a sanitizing layer 110. In some embodiments, sanitizing agents 120 having adequate vapor pressures to cause the sanitizing agents 120 to evaporate at room temperature are provided. In some embodiments, sanitizing agents 120 having adequate vapor pressures to cause the sanitizing agent 120 to evaporate at human body temperature are provided. Numerous such sanitizing agents 120 are known and have been described herein.

In some embodiments, release of a sanitizing agent 120 from a substantially continually sanitizing surface 112 through evaporation may be controlled by the characteristics of the sanitizing layer 110, such as the density of the layer, the thickness of the layer, the characteristics of pores through the layer, the permeability of the layer, and the like. For example, the rate of release of one or more sanitizing agents 120 may be controlled by the size and shape of pores associated with the sanitizing layer 110, the thickness of the sanitizing layer 110, the temperature of the sanitizing layer 110, and the like.

In some embodiments, release of a sanitizing agent 120 from a substantially continually sanitizing surface 112 through evaporation may be controlled according to diffusion of the sanitizing agent 120 through the sanitizing layer 110. For example, the rate of release of one or more sanitizing agents 120 may be controlled by the permeability of the layer or layers used to prepare the sanitizing layer 110, the thickness of the sanitizing layer 110, the temperature of the sanitizing layer 110, and the like.

In some embodiments, release of a sanitizing agent 120 from a substantially continually sanitizing surface 112 through evaporation may be controlled through formulation of the sanitizing agent 120. For example, a sanitizing agent 120 may be formulated with carriers having high vapor pressures at room temperature to increase release of the sanitizing agent 120 as compared to the release of a sanitizing agent 120 that was formulated with a carrier having low vapor pressure at room temperature. Accordingly, sanitizing agents 120 may be formulated in numerous forms to control release of the sanitizing agent 120 through evaporation.

At embodiment 404, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through sublimation.

Sanitizing agents 120 that are able to transition from the solid phase to the gaseous phase may be used in association with a sanitizing layer 110. In some embodiments, sanitizing agents 120 having adequate vapor pressures to cause the sanitizing agents 120 to sublimate at room temperature are provided. In some embodiments, sanitizing agents 120 having adequate vapor pressures to cause the sanitizing agent 120 to sublimate at human body temperature are provided. Such sanitizing agents 120 include, but are not limited to, halogens, halogen compounds, molecular iodine, 1,4-dichlorobenzene, and the like (i.e., U.S. Pat. No.: 5,733,270; herein incorporated by reference).

In some embodiments, release of a sanitizing agent 120 from a substantially continually sanitizing surface 112 through sublimation may be controlled by the characteristics of the sanitizing layer 110, such as the density of the layer, the thickness of the layer, the characteristics of pores through the layer, the permeability of the layer, and the like. For example, the rate of release of one or more sanitizing agents 120 may be controlled by the size and shape of pores associated with the sanitizing layer 110, the thickness of the sanitizing layer 110, the temperature of the sanitizing layer 110, and the like.

In some embodiments, release of a sanitizing agent 120 from a substantially continually sanitizing surface 112 through sublimation may be controlled according to diffusion of the sanitizing agent 120 through the sanitizing layer 110. For example, the rate of release of one or more sanitizing agents 120 may be controlled by the permeability of the layer or layers used to prepare the sanitizing layer 110, the thickness of the sanitizing layer 110, the temperature of the sanitizing layer 110, and the like.

At embodiment 406, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through physical dissociation.

In some embodiments, one or more substantially continually sanitizing surfaces 112 may be constructed such that one or more sanitizing agents 120 may physically dissociate. For example, in some embodiments, a substantially continually sanitizing surface 112 may be constructed with particles that are held within a dissociable matrix. As the matrix dissociates, particles that are held within the matrix will dissociate from the substantially continually sanitizing surface 112 to expose a sanitized surface. Matrices may dissociate through numerous mechanisms that include, but are not limited to, physical degradation, sublimation, oxidation mediated degradation, light mediated degradation, and the like.

Particles that may be included within a substantially continually sanitizing surface 112 may be of numerous shapes. In some embodiments, particles may be substantially uniform in shape. In some embodiments, particles may vary substantially in shape. For example, particles may be beads, flakes, disks, threads, rods, circles, ovals, ellipses, triangles, squares, rectangles, pentagons, hexagons, stars, barbells, and the like. In some embodiments, particle shape may be selected with regard to the substance or substances used to construct the matrix. Particle shapes may also be selected such that the rate of dissociation and/or conditions under which the particles will dissociate may be controlled.

Particles that may be included within a substantially continually sanitizing surface 112 may be of numerous sizes. In some embodiments, particles may be substantially uniform in size. In some embodiments, particles may vary substantially in size. For example, in some embodiments, particles may be within a range of about 1 nanometer to about 1 centimeter. In some embodiments, particles may be within a range of about 1 millimeter to about 1 centimeter. In some embodiments, particles may be within a range of about 1 millimeter to about 10 millimeters. In some embodiments, particles may be within a range of about 1 millimeter to about 5 millimeters. In some embodiments, particle size may be selected with regard to the substance or substances used to construct the matrix. Particle sizes may also be selected such that the rate of dissociation and/or conditions under which the particles will dissociate may be controlled. In some embodiments, particles may be selected such that they are able to be cleared from an area through normal air circulation. For example, in some embodiments, particles may essentially float as airborne particles that can be collected within filters present in the heating and cooling network associated with the area. In some embodiments, particles may be selected that will fall to the ground following dissociation from a substantially continually sanitizing surface 112.

Numerous substances may be used to produce particles for inclusion in a substantially continually sanitizing surface 112. Examples of such substances include, but are not limited to, silica, plastic, metal, polymeric substances, and the like.

Numerous substances may be used to produce a matrix that will dissociate over time and release particles contained within the matrix. In some embodiments, a substance may be used to produce a matrix that will dissociate through sublimation. For example, in some embodiments, particles may be included within a 1,4-dichlorobenzene matrix that will sublimate over time and cause the particles contained within the matrix to dissociate.

In some embodiments, matrices may be produced that undergo light-mediated dissociation. For example, in some embodiments, matrices may be produced from short polymers that are cross-linked with photocleavable linkers. Accordingly, as the photocleavable cross-linkers are cleaved through the action of light, the short polymers are able to dissociate and free particles that are included within the polymeric matrix. Photocleavable substances are known and have been described (i.e., U.S. Pat. Nos.: 6,806,361; 5,563,238; 5,360,892; 4,197,375; 4,073,764; 4,042,765; and 4,476,255; hereby incorporated by reference). In some embodiments, an ultraviolet light degradable polymer may be a polylactic acid polymer that includes a copolymer of polylactic acid and a modifying monomer. Such modifying monomers include, but are not limited to, p-dioxanone, 1,5 dioxepan-2-one, and 1,4 oxathialan-2-one, 4,4 dioxide, or mixtures thereof (U.S. Pat. No. 5,563,238; hereby incorporated by reference). In some embodiments, matrices may be produced that undergo oxidation-mediated dissociation. For example, in some embodiments, matrices may be produced from short polymers that are cross-linked with linkers that may be cleaved through atmosphere-mediated oxidation. Accordingly, oxidation-mediated cleavage of the cross-linkers retaining the short polymers may allow the polymers to dissociate and free particles that are included within the polymeric matrix.

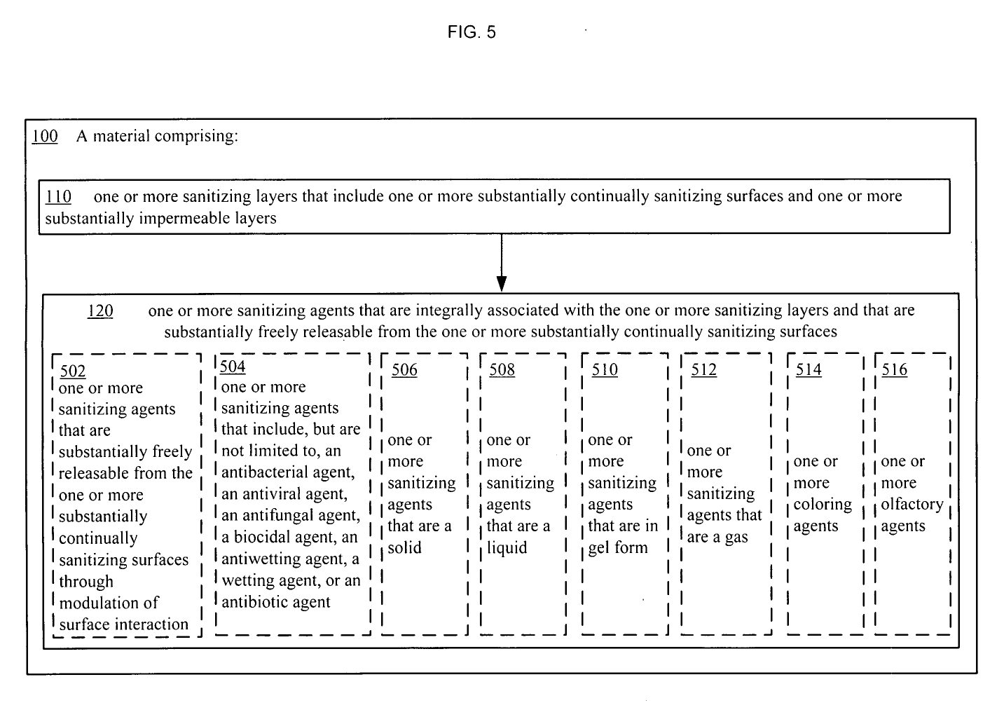

FIG. 5 illustrates alternative embodiments of the material 100 of FIG. 1. FIG. 5 illustrates example embodiments of the one or more sanitizing agents that are integrally associated with the one or more sanitizing layers and that are substantially freely releasable from the one or more substantially continually sanitizing surfaces 120. Additional embodiments may include embodiment 502, embodiment 504, embodiment 506, embodiment 508, embodiment 510, embodiment 512, embodiment 514, and/or embodiment 516.

At embodiment 502, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through modulation of surface interaction.

In some embodiments, the interaction of a substantially continually sanitizing surface 112 and one or more sanitizing agents 120 may be regulated to control release of the one or more sanitizing agents 120 from the sanitizing surface 112. For example, in some embodiments, one or more sanitizing surfaces 112 may be constructed such that they provide for low energy release of one or more sanitizing agents 120. Such sanitizing surfaces 112 may include silicone rubber, polytetrafluoroethylene, or other substances which are known to exhibit good release properties (i.e., U.S. Pat. No.: 5,779,795; herein incorporated by reference).

In some embodiments, the surface interaction of a sanitizing surface 112 and one or more sanitizing agents 120 may be modulated through selecting a combination of one or more sanitizing agents 120 and sanitizing surfaces 112 based upon the physical and chemical properties of the sanitizing agents 120 and sanitizing surfaces 112. For example, in some embodiments, release of a sanitizing agent 120 from a sanitizing surface 112 may be decreased by selecting a sanitizing agent 120 and a sanitizing surface 112 that are chemically attracted to each other. Examples of such embodiments include those where a hydrophilic sanitizing agent is paired with a hydrophilic sanitizing surface or where a hydrophobic sanitizing agent is paired with a hydrophobic sanitizing surface. In some embodiments, release of a sanitizing agent 120 from a sanitizing surface 112 may be increased by selecting a sanitizing agent 120 and a sanitizing surface 112 that are chemically repelled from each other. Examples of such embodiments include those where a hydrophilic sanitizing agent 120 is paired with a hydrophobic sanitizing surface 112 or where a hydrophobic sanitizing agent 120 is paired with a hydrophilic sanitizing surface 112. Accordingly, numerous combinations of sanitizing agents 120 can be paired with numerous combinations of sanitizing surfaces 112 to control release of a sanitizing agent 120 from a sanitizing surface 112.

In some embodiments, one or more sanitizing agents 120 may be selected based on their physical and chemical properties to control release of the one or more sanitizing agents 120 from a sanitizing surface 120. In some embodiments, one or more sanitizing agents 120 may be mixed with other substances to form mixtures having selected physical and chemical properties that control release of the one or more sanitizing agents 120 from a sanitizing surface 120. In some embodiments, one or more sanitizing agents 120 may be selected based on the surface tension that they exhibit while on the sanitizing surface 112 to control release of the one or more sanitizing agents 120. In some embodiments, one or more sanitizing agents 120 may be mixed with high molecular weight molecules that allow the resulting mixture to be self-siphoning to facilitate delivery of the sanitizing agents 120 to a sanitizing surface 112. In some embodiments, one or more sanitizing agents 120 may be mixed with fluids that exhibit non-Newtonian characteristics to control release of the sanitizing agents 120 from a sanitizing surface 112. In some embodiments, the electrochemical characteristics of a sanitizing agent 120 or a mixture that includes a sanitizing agent 120 may be used to control release of the sanitizing agent 120 from a sanitizing surface 112.

In some embodiments, substances which are known to exhibit good release properties may be combined with porous and/or permeable layers to provide for movement of one or more sanitizing agents 120 through the porous and/or permeable layers to the sanitizing surface 112 where they are released. Movement of the one or more sanitizing agents 120 through the porous and/or permeable layers may occur through capillary action, wicking, use of propellants, or numerous other modalities. For example, in some embodiments, a sanitizing material 100 may include a porous layer that is adhered to a control layer and a release layer that is adhered to the control layer. The release layer may serve as the sanitizing surface 112 from which one or more sanitizing agents 120 may be released. The porous layer may include an open-celled thermosetting polymer foam that may optionally be internally reinforced. In some embodiments, the porous layer may have high compatibility with, and wettability by, the one or more sanitizing agents 120 and have high liquid holding capacity to provide for smooth substantially continuous delivery of the one or more sanitizing agents 120. In some embodiments, the control layer may include a porous polytetrafluoroethylene film in which the pores contain a mixture of silicone oil and silicone rubber. In some embodiments, the release layer may include a porous polytetrafluoroethylene film. Methods to create such films are known (i.e., U.S. Pat. No.: 5,779,795; herein incorporated by reference).

At embodiment 504, the one or more sanitizing agents that are integrally associated with the one or more sanitizing layers and that are substantially freely releasable from the one or more substantially continually sanitizing surfaces may include one or more sanitizing agents that include, but are not limited to, an antibacterial agent, an antiviral agent, an antifungal agent, a biocidal agent, an antiwetting agent, a wetting agent, or an antibiotic agent. Examples of such sanitizing agents 120 are known and have been described (i.e., The Merck Index: An Encyclopedia of Chemicals, Drugs, and Biologicals, Whitehouse Station, N.J., 13^{th} Edition, 2001).

At embodiment 506, the one or more sanitizing agents that are integrally associated with the one or more sanitizing layers and that are substantially freely releasable from the one or more substantially continually sanitizing surfaces may include one or more sanitizing agents that are a solid.

Numerous solid sanitizing agents 120 are known and have been described herein. Such sanitizing agents 120 include, but are not limited to, complexes of polyvinylpyrrolidone (PVP) and H₂O₂, 1,4-dichlorobenzene, complexes of iodine, and the like (i.e., U.S. Pat. No. 5,008,106; hereby incorporated by reference). In some embodiments, one or more solid sanitizing agents 120 that dissolve upon contact with a liquid such as water may be associated with a sanitizing layer 110. Examples of such sanitizing agents 120 include, but are not limited to, sodium dodecyl sulfate, lithium sulfate, lauric acid, and salts thereof. In some embodiments, such sanitizing agents 120 may be used at one or more concentrations and under conditions where the sanitizing agents 120 will exhibit antiviral activity. In some embodiments, such sanitizing agents 120 may be used at one or more concentrations and under conditions where the sanitizing agents 120 will exhibit a spermicidal activity. Accordingly, in some embodiments, substances are provided that may be used in the manufacture of prophylactic devices (i.e., U.S. Pat. No. 6,192,887; herein incorporated by reference).

At embodiment 508, the one or more sanitizing agents that are integrally associated with the one or more sanitizing layers and that are substantially freely releasable from the one or more substantially continually sanitizing surfaces may include one or more sanitizing agents that are a liquid.

Numerous liquid sanitizing agents 120 are known and have been described herein. Such sanitizing agents 120 include, but are not limited to, hydrogen peroxide, alcohols, detergents, solutions of sanitizing agents 120, and the like (i.e., U.S. Pat. Nos.: 4,642,165; 4,744,951; 5,008,106; herein incorporated by reference). In some embodiments, one or more sanitizing agents 120 may form a solution that exhibits sanitizing activity. For example, in some embodiments, a sanitizing agent 120 may include a water soluble salt (i.e., sodium carbonate) in combination with an anti-microbial agent (i.e., sulfur) that dissolves upon exposure to water and forms an alkaline solution that dissolves some sulfur that kills bacteria (i.e., U.S. Pat. No.: 2,216,333; herein incorporated by reference).

TABLE I
Examples of liquid sanitizing agents 120 that may
be included in sanitizing surfaces

| | Known Minimum | |
|---|---|---|
| | Concentration by | Known Minimum |
| | Volume for Immediately | Concentration by |
| | Sterilizing Human | Volume for |
| Chemical | Immunodeficiency | Immediately Sterilizing |
| Compound | Virus (HIV-1) | Hepatitis B Virus |
| Ethyl Alcohol | 50% | 80% |
| Isopropyl Alcohol | 30% | 70% |
| NP-40 (ethylphenyl- | 1% | — |
| polyethylene glycol) | | |
| Hydrogen Peroxide | 0.3% | — |
| Household Bleach | 0.1% | 10% |

At embodiment 510, the one or more sanitizing agents that are integrally associated with the one or more sanitizing layers and that are substantially freely releasable from the one or more substantially continually sanitizing surfaces may include one or more sanitizing agents that are in gel form.

In some embodiments, one or more sanitizing agents 120 may be prepared in gel form. The viscosity of such gels may be varied to control the rate at which the sanitizing agents 120 are released from one or more sanitizing surfaces 112. For example, in some embodiments, one or more sanitizing agents 120 may be prepared in gel form having very low viscosity to provide for rapid flow through a porous layer 204 and release from a sanitizing surface 112 while in other embodiments, one or more sanitizing agents 120 may be prepared in gel form having very high viscosity to provide for slow flow through a porous layer 204 and slower release from a sanitizing layer 110. In some embodiments, one or more sanitizing agents 120 may be prepared in gel form to alter the vapor pressure associated with the sanitizing agent 120.

Gels may be made by combining one or more sanitizing agents 120 with one or more viscosity-modifying substances. Examples of such substances include, but are not limited to, xantham gum, gum acacia, gum tragacanth, agar, glycyrrhiza, polyvinylpyrrolidone polymers having an average molecular weight between about 500 to about 5000 grams/mole, cross-linked polyvinylpyrrolidone polymers, sodium alginate NF, pectin NF from citrus fruit or apple pomace, other plant gums, theobroma oil (also known as cacao butter or cocoa butter), cellulose, methyl cellulose (Methocel, trademark of Dow Chemical Co.) carboxymethylcellulose (CMC) sodium, hydroxyethyl cellulose (Cellosize, trademark of The Carbide and Carbon Chemicals Corp.), hydroxpropylmethylcelluloses designated Methocel 60, HG, Methocel HG65, Methocel HG70, Methocel HG90 (wherein the number refers to the approximate gel point of a 2 percent solution), other alkylated celluloses including ethylcellulose, hydroxyethyl cellulose, propylcellulose, microcrystalline cellulose (Avicel PH, trademark of FMC Corporation, Philadelphia, Pa.), other suitable chemically-modified celluloses, glycerol, propylene glycol, pyroxylin, polyethylene glycols of between about 150 to more than about 6000 molecular weight, polyethylene glycol 400, polyethylene glycol 4000, polyethylene glycol 6000, gelatin A, gelatin B, glycinerated gelatin, wool fat, beeswax, White petrolatum USP, Petrolatum NF (Petroleum Jelly with a melting point of 42-60° C.), Plastibase (tradename “Plastibase” from E.R. Squibb & Co., also called Jelene: a combination of mineral oils and heavy hydrocarbon waxes with a molecular weight of about 1300), anhydrous lanolin USP, microcrystalline wax, cholesterol, white wax, hard paraffin wax, yellow soft paraffin wax, white soft paraffin wax, sodium lauryl sulfate, stearyl alcohol, carbowax polyethylene glycol 1000, carbowax polyethylene glycol 1500, carbowax polyethylene glycol 1540, carbowax polyethylene glycol 4000, carbowax polyethylene glycol 6000, dimethicones including those more than 1000 centistokes in viscosity, simethicone, dimethylpolysiloxane, perfluropolymethylisopropyl ethers of 1000 to more than 6600 molecular weight, starch, other alkylated starches, other chemically-modified starches, bentonite USP, sodium bentonite, potassium bentonite, calcium bentonite, magnesium bentonite, hydrogen bentonite, Voloclay bentonite (a combination of sodium bentonite, potassium bentonite, calcium bentonite, magnesium bentonite, and hydrogen bentonite), attapulgite (a hydrous magnesium aluminum silicate that is heat-activated), Veegum (a colloidal magnesium aluminum silicate), carbopol 934 at neutral pH (a trademark acidic polymer of B.F. Goodrich Chemical Co.), benzoinated lard, guar gum, agar, pulverized natural sponge, potato starch, corn starch, other vegetable starches, other plant cellulose fibers, other plant or mineral fibers or polymers, other synthetic polymers, and mixtures thereof.

At embodiment 512, the one or more sanitizing agents that are integrally associated with the one or more sanitizing layers and that are substantially freely releasable from the one or more substantially continually sanitizing surfaces may include one or more sanitizing agents that are a gas.

In some embodiments, a gas may include a vapor. In some embodiments, hydrogen peroxide may be released from the one or more substantially continually sanitizing surfaces 112. Hydrogen peroxide may be generated from peroxide compounds. Examples of such peroxide compounds include, but are not limited to, hydrogen peroxide, urea hydrogen peroxide, benzoyl peroxide, succinyl peroxide, barium peroxide, calcium peroxide, magnesium peroxide, sodium peroxide, strontium peroxide, zinc peroxide, other peroxide compounds, and mixtures thereof. When exposed to fine metal powders or metal oxide catalysts such as manganese dioxide, the peroxides in aqueous solutions buffered at neutral pH or alkaline pH generally can release oxygen gas or can form hydrogen peroxide (U.S. Pat. Nos.: 5,357,636; 4,169,123; 4,169,124; 4,643,876; 4,943,414; herein incorporated by reference).

In some embodiments, halogens and halogenated compounds may be reacted in aqueous or aqueous/alcoholic solutions with an acid to release sanitizing agents 120 in soluble and gaseous forms. Examples of such halogens include fluorine, chlorine, bromine and iodine.

At embodiment 514, the one or more sanitizing agents that are integrally associated with the one or more sanitizing layers and that are substantially freely releasable from the one or more substantially continually sanitizing surfaces may include one or more coloring agents.

Numerous coloring agents, such as pigments, dyes, and other indicators are known and have been described (i.e., U.S. Pat. Nos.: 7,101,408; 5,403,363; 4,855,413; 4,855,412; 4,774,324; 4,500,455; 4,325,870; 4,208,324; herein incorporated by reference). In some embodiments, one or more coloring agents 514 may be released upon depletion of one or more sanitizing agents 120. In some embodiments, such coloring agents 514 may be released in association with one or more sanitizing agents 120 to indicate the presence of the one or more sanitizing agents 120. In some embodiments, coloring agents 514 may be visible to the unaided human eye. In some embodiments, coloring agents 514 may be invisible to the unaided human eye and may be detected through use of instrumentation. In some embodiments, the presence or absence of one or more coloring agents 514 may be visible as a color change. In some embodiments, the presence or absence of one or more coloring agents 514 may be discernible through substantially any change visible to the unaided human eye. For example, in some embodiments, one or more coloring agents 514 may be iridescent.

At embodiment 516, the one or more sanitizing agents that are integrally associated with the one or more sanitizing layers and that are substantially freely releasable from the one or more substantially continually sanitizing surfaces may include one or more olfactory agents.

In some embodiments, a sanitizing agent 120 may include a chemical odor or odorant that is capable of causing either a pleasant or an unpleasant (malodorous) smell. Numerous odorants may be utilized. Examples of such odorants include, but are not limited to, aromatic oils, perfumes, esters, ketones, aldehydes, organic acids, sulfides, amines, flower extracts, plant extracts, animal extracts, mineral extracts, or any other suitable chemical. For example a sanitizing agent 120 may include a pleasant scented volatile oil such as peppermint oil, menthol, oil of wintergreen, lemon oil and the like, or an unpleasant odor such as pyridine, putrescene, ammonia, vinegar, formaldehyde, and the like. In some embodiments, an odor will be present when the sanitizing agent 120 is present. In some embodiments, an odor will appear when the sanitizing agent 120 is not present. Accordingly, odor can act to indicate the presence or absence of a sanitizing agent 120.

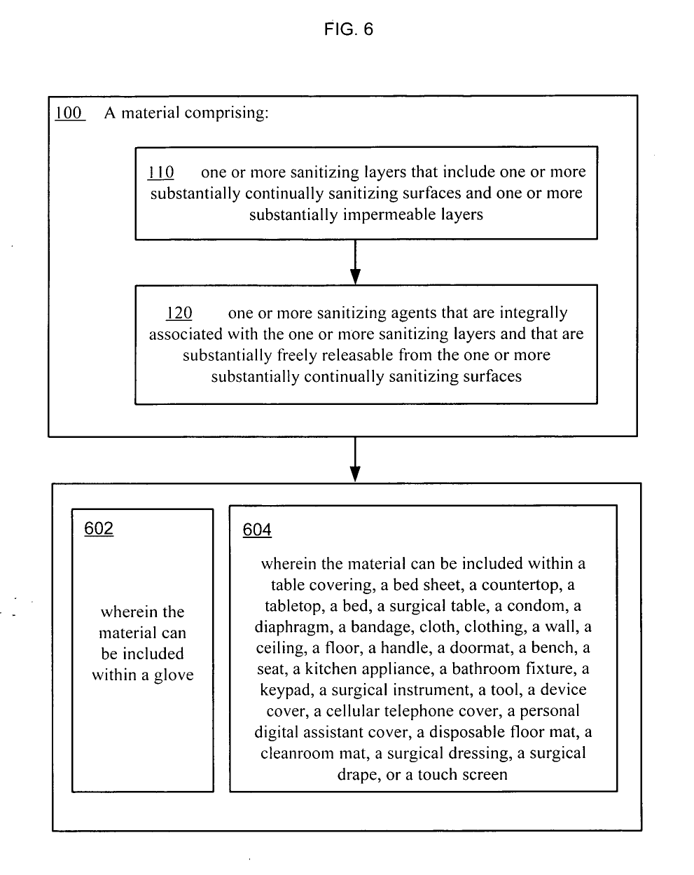

FIG. 6 illustrates alternative embodiments of the material 100 of FIG. 1. FIG. 6 illustrates example embodiments of the material 100. Additional embodiments may include embodiment 602, and/or embodiment 604.

At embodiment 602, the material can be included within a glove. Examples of such gloves include, but are not limited to, surgical gloves, examination gloves, gloves used in butchering facilities, utility gloves, gauntlet gloves, veterinary gloves, and the like. In some embodiments, gloves may cover an individual's hands. In some embodiments, gloves may cover an individual's hands and wrists. In some embodiments, gloves may cover an individual's hands and portions of their associated arm. For example, in some embodiments, gloves may cover an individual's hands and arms to the shoulder, such as with a veterinary sleeve. In other embodiments, a glove may cover an individual's hand and a portion of their associated arm, such as their wrist, forearm, elbow, upper arm, and substantially any combination thereof.

At embodiment 604, the material can be included within a table covering, a bed sheet, a countertop, a tabletop, a bed, a surgical table, a condom, a diaphragm, a bandage, cloth, clothing, a wall, a ceiling, a floor, a handle, a doormat, a bench, a seat, a kitchen appliance, a bathroom fixture, a keypad, a surgical instrument, a tool, a device cover, a cellular telephone cover, a personal digital assistant cover, a disposable floor mat, a cleanroom mat, a surgical dressing, a surgical drape, or a touch screen.

In some embodiments, a material 100 may be used to cover an object that may exposed to contamination. Examples of such objects include, but are not limited to, door handles, tools, portable devices, and the like. In some embodiments, a material 100 may be used to cover one or more persons. For example, in some embodiments, a material 100 may be included within a surgical drape, such as a surgical area protective drape and the like. In some embodiments, a material 100 may be included within a surgical dressing, such as a surgical field dressing and the like. In some embodiments, such surgical drapes and dressings may be used to maintain positive sterility in the immediate vicinity of a surgical wound or aperture.

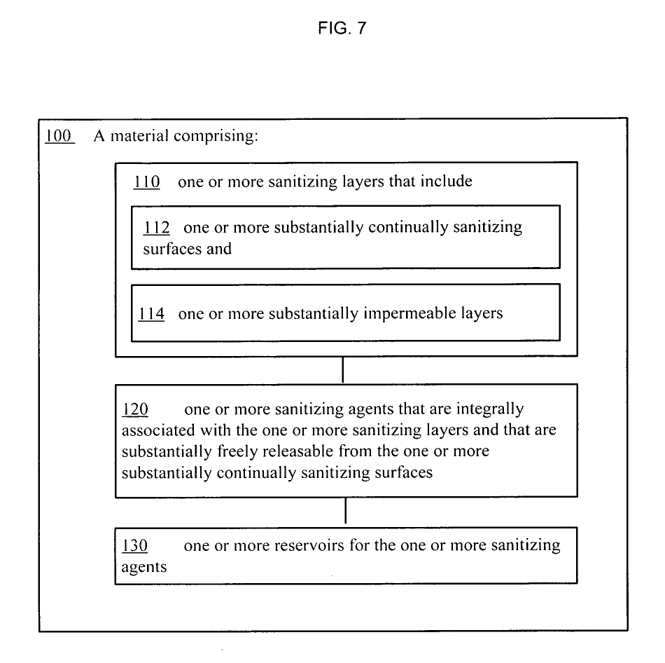

FIG. 7 represents a material 100 that may include one or more sanitizing layers that include one or more substantially continually sanitizing surfaces and one or more substantially impermeable layers 110, one or more sanitizing agents that are integrally associated with the one or more sanitizing layers and that are substantially freely releasable from the one or more substantially continually sanitizing surfaces, and one or more reservoirs for the one or more sanitizing agents.

In some embodiments, a sanitizing layer 110 may include one or more reservoirs 130. In some embodiments, such reservoirs 130 may contain one type of sanitizing agent 120. In some embodiments, such reservoirs 130 may contain one or more types of sanitizing agents 120. In some embodiments, such reservoirs 130 may contain components other than sanitizing agents 120. For example, such reservoirs 130 may contain coloring agents 514, olfactory agents 516, and the like.

In some embodiments, reservoirs 130 may be integrally associated with a sanitizing layer 110. For example, in some embodiments, a reservoir 130 may be contained within a sanitizing layer 110 that opens onto the surface of the sanitizing layer 110. In other embodiments, a sanitizing layer 110 may include a reservoir 130 that connects to a channel 210 that opens onto the surface of the sanitizing layer 110. In some embodiments, a material 100 may include one or more reservoirs 130 that may be connected to one or more sanitizing layers 110 through a connection, such as tubing. Accordingly, reservoirs 130 may be configured in numerous geometries.

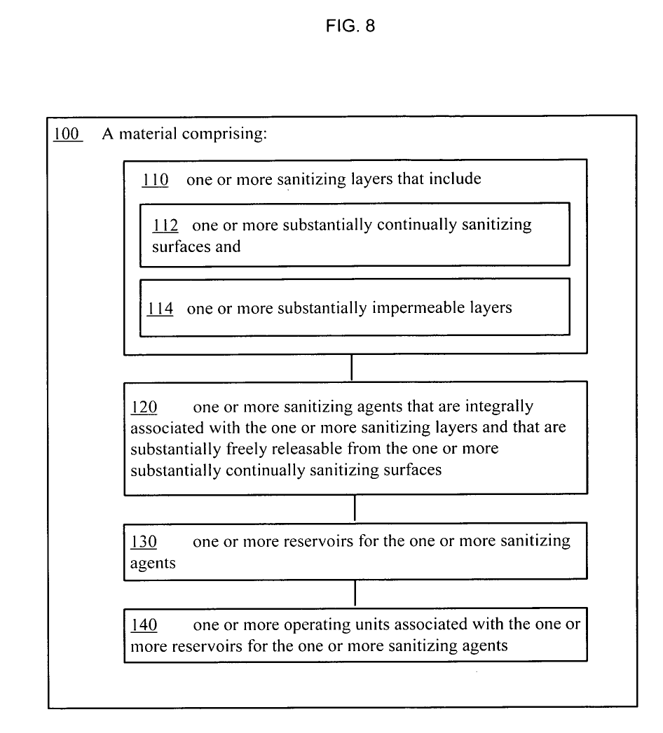

FIG. 8 represents a material 100 that may include one or more sanitizing layers that include one or more substantially continually sanitizing surfaces and one or more substantially impermeable layers, one or more sanitizing agents that are integrally associated with the one or more sanitizing layers and that are substantially freely releasable from the one or more substantially continually sanitizing surfaces, one or more reservoirs for the one or more sanitizing agents, and one or more operating units associated with the one or more reservoirs for the one or more sanitizing agents.

In some embodiments, one or more operating units 140 may be associated with one or more reservoirs 130 that may include one or more sanitizing agents 120. In some embodiments, the one or more reservoirs 130 may be integrally associated with the one or more sanitizing layers 110. In some embodiments, the one or more reservoirs 130 may be remotely associated with the one or more sanitizing layers 110 through use of a connector, such as a tube. In some embodiments, an operating unit 140 may control the transport of one or more sanitizing agents 120 from one or more reservoirs 130 to one or more sanitizing layers 110. In some embodiments, an operating unit 140 may control the transport of one or more sanitizing agents 120 from one or more reservoirs 130 to one or more substantially continually sanitizing surfaces 112. For example, in some embodiments, an operating unit 140 may include a pump that propels one or more sanitizing agents 120 from one or more reservoirs 130. In some embodiments, an operating unit 140 may include one or more valves that control the release of one or more sanitizing agents 120 from one or more reservoirs 130. Accordingly, numerous configurations of hardware and software may be included within an operating unit 140 to control transport of one or more sanitizing agents 120 from one or more reservoirs 130. In some embodiments, one or more operating units 140 may receive one or more signals that include one or more instructions that control the actions of the operating unit 140. For example, in some embodiments, an operating unit 140 may receive a signal to propel one or more sanitizing agents 120 from one or more reservoirs 130 to a sanitizing layer 110 and/or to a substantially continually sanitizing surface 112. In some embodiments, an operating unit 140 may receive a signal to stop propelling one or more sanitizing agents 120 from one or more reservoirs 130 to a sanitizing layer 110 and/or to a substantially continually sanitizing surface 112. Accordingly, in some embodiments, one or more operating units 140 may be programmed to cause a surface to be sanitized according to virtually any set of instructions. In some embodiments, the one or more operating units 140 may include control features that provide for user interaction with the operating units 140 through one or more user interfaces 150. In some embodiments, user interaction may occur through direct interaction with an operating unit 140 through one or more user interfaces 150 that may include, but are not limited to, switches, buttons, touch pads, levers, and the like. In some embodiments, user interaction may occur through indirect interaction with an operating unit 140 through one or more user interfaces 150 that include, but are not limited to, a wireless connection, an internet connection, a hardwired connection, and the like. In some embodiments, one or more operating units 140 may be able to interact with additional devices. For example, in some embodiments, an operating unit 140 may receive a signal from another device to sanitize a surface when material 100 associated with the operating unit 140 enters or leaves an area. One example of such an instance would be when a surgical drape that includes a material 100 having a sanitizing surface 112 enters into an operating room, an associated operating unit 140 may receive one or more signals to sanitize the surgical drape. Accordingly, numerous other configurations and control devices may be associated with an operating unit 140. Such configurations and devices include those described within U.S. patent application Ser. No. 11/414,743, entitled METHODS AND SYSTEMS FOR MONITORING STERILIZATION STATUS; and U.S. patent application Ser. No. 11/440,460, entitled METHODS AND SYSTEMS FOR STERILIZATION; herein incorporated by reference. Accordingly, in some embodiments, a material 100 may include a sterilization indicator as described as described within U.S. patent application Ser. No. 11/440,460.

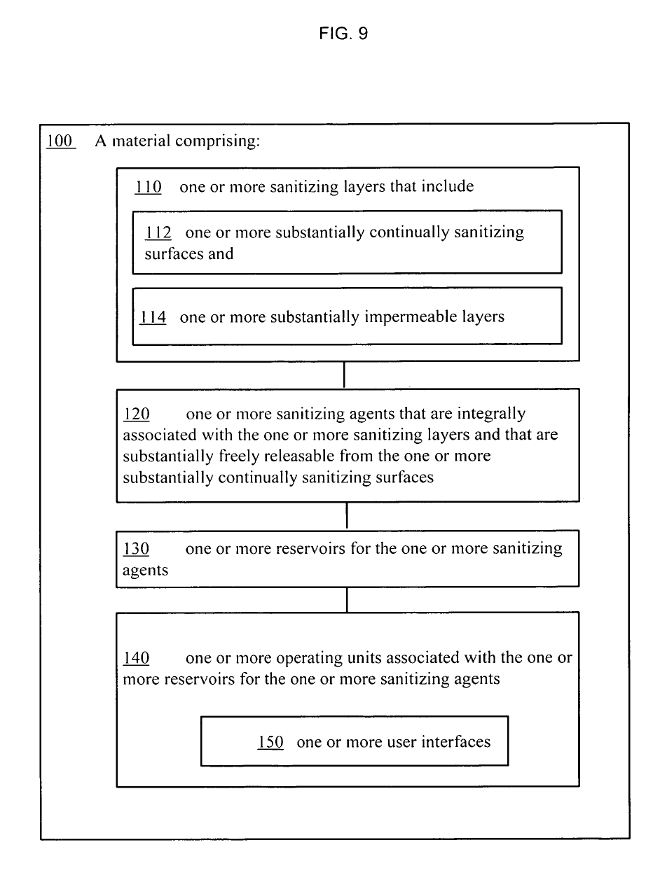

FIG. 9 represents a material 100 that may include one or more sanitizing layers that include one or more substantially continually sanitizing surfaces and one or more substantially impermeable layers, one or more sanitizing agents that are integrally associated with the one or more sanitizing layers and that are substantially freely releasable from the one or more substantially continually sanitizing surfaces, one or more reservoirs for the one or more sanitizing agents, one or more operating units associated with the one or more reservoirs for the one or more sanitizing agents, and the one or more operating units provide one or more user interfaces.

In some embodiments, one or more users may interact with one or more operating units 140 through one or more user interfaces 150. Such user interaction can include, but is not limited to, controlling an operating unit 140 with regard to parameters associated with the operating unit 140, a sanitizing layer 110, and/or a sanitizing agent 120. Parameters may include, but are not limited to, the time, place, duration, intensity, priority, and/or identity of one or more sanitizing agents 120 that are used to sanitize a sanitizing layer 110.

User interaction may occur directly or indirectly. For example, in some embodiments, a user may directly interact with an operating unit 140 through use of one or more user interfaces 150 that include, but are not limited to, switches, levers, buttons, a keyboard, a touchpad, and the like. In some embodiments, a user may interact with an operating unit 140 indirectly by transmitting one or more signals from one or more user interfaces 150 that are received by an operating unit 140 that control transport of a sanitizing agent 120 to a sanitizing layer 110. In some embodiments, a user is human. In some embodiments, a user is not human.

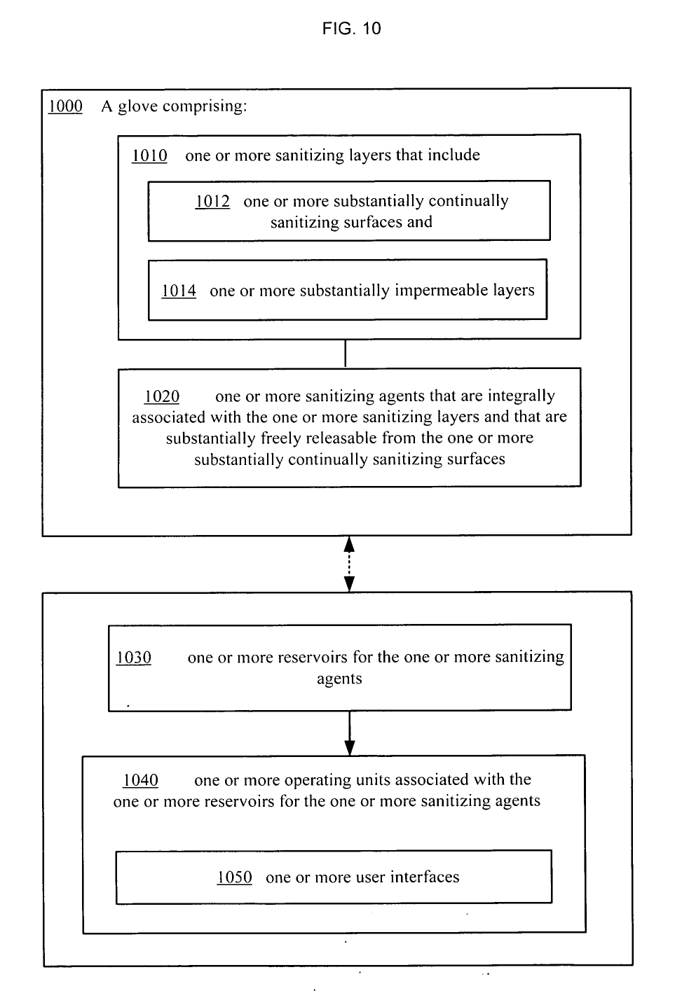

FIG. 10 represents a glove 1000 that may include one or more sanitizing layers that include one or more substantially continually sanitizing surfaces and one or more substantially impermeable layers 1010.

A sanitizing layer 1010 that includes one or more substantially continually sanitizing surfaces 1012 is a layer from which one or more sanitizing agents 1020 may be substantially freely releasable from one or more substantially continually sanitizing surfaces 1012. In some embodiments, one or more sanitizing agents 1020 may be substantially continually freely released until the supply of the sanitizing agents 1020 is exhausted. In some embodiments, one or more sanitizing agents 1020 may be substantially continually freely released on an intermittent basis. For example, in some embodiments, sanitizing agents 1020 may be substantially continually freely released for a period of time, after which the release of the sanitizing agents 1020 may be halted for a period of time, and then the release of the sanitizing agent 1020 may be resumed. In some embodiments, the one or more sanitizing agents 1020 may be prevented from being released from a substantially continually sanitizing surface 1012 until release is initiated whereupon the one or more sanitizing agents 1020 may be substantially continually freely released.

In some embodiments, a sanitizing layer 1010 may include one or more portions that release a first type of sanitizing agent 1020 and one or more portions that release a second type of sanitizing agent 1020 that is different that the first type of sanitizing agent 1020. In some embodiments, a sanitizing layer 1010 may include one or more portions that release one or more sanitizing agents 1020 at a first time point and one or more portions that release one or more sanitizing agents 1020 at one or more time points that are different from the first time point. Accordingly, in some embodiments, sanitizing layers 1010 may include portions from which the same or different sanitizing agents 1020 are released, one or more portions from which sanitizing agents 1020 are released at different times, one or more portions from which one or more sanitizing agents 1020 are released with different intensities, and substantially any combination thereof.

Numerous substances may be used alone or in combination with other substances to prepare sanitizing layers 1010. Examples of such substances include, but are not limited to, polymeric substances, sintered polymers, metals, and ceramics; non-wovens, such as Tyvex® (high density polyethylene); microporous membranes; track etched membranes; dense film structures such as polyesters, thermoplastic elastomers, and low density polyolefins, and the like.

Examples of such polymeric substances include, but are not limited to, latex rubber, polyisoprene, neoprene rubber, polybutadiene, and silicone rubber. Examples of monomers which may be used include, but are not limited to, hydroxy alkyl esters of alpha, beta-unsaturated carboxylic acids (i.e., 2-hydroxy ethylacrylate, 2-hydroxy methacrylate, hydroxypropylacrylate, methacrylate, and the like). Many derivatives of acrylic or methacrylic acid may be used to form polymers. Examples of these include, but are not limited to, dimethylaminoethyl methacrylate, piperidinoethyl methacrylate, morpholinoethyl methacrylate, methacrylylglycolic acid, methacrylic acid, the monomethacrylates of glycol, glycerol, monomethacrylates of dialkylene glycols, polyalkylene glycols, and the like. In some embodiments, acrylates may be substituted for the corresponding methacrylates. Additional examples of monomers which may be used include, but are not limited to, 2-hydroxyethyl acrylate, 2-hydroxyethyl methacrylate, diethylene glycol acrylate, diethylene glycol methacrylate, 2-hydroxypropyl acrylate, 2-hydroxypropyl methacrylate, 3-hydroxypropyl acrylate, 3-hydroxypropyl methacrylate, tetraethyleneglycol acrylate, tetraethyleneglycol methacrylate, pentaethyleneglycol acrylate, pentaethyleneglycol methacrylate, dipropyleneglycol acrylate, dipropyleneglycol methacrylate, acrylamide, methacrylamide, diacetone acrylamide, methylolacrylamide, methylolmethacrylanide, acrylic acid, methacrylic acid, itaconic acid, aconitic acid, cinnamic acid, crotonic acid, mesaconic acid, maleic acid, fumaric acid, mono-2-hydroxypropyl aconitate, mono-2-hydroxyethyl maleate, mono-2-hydroxypropyl fumarate, mono-ethyl itaconate, monomethyl cellosolve ester of itaconic acid, monomethyl cellosolve ester of maleic acid, diethylaminoethyl acrylate, diethylaminoethyl methacrylate, dimethylaminoethyl acrylate, dimethylaminoethyl methacrylate, monoethylaminoethyl acrylate, monoethylaminoethyl methacrylate, tert. butylaminoethyl methacrylate, para-amino styrene, ortho-amino styrene, 2-amino-4-vinyl toluene, piperidinoethyl methacrylate, morpholinoethyl methacrylate, 2-vinyl pyridine, 3-vinyl pyridine, 4-vinyl pyridine, 2-ethyl-5-vinyl pyridine, dimethylaminopropyl acrylate, dimethylaminopropyl methacrylate, dimethylaminoethyl vinyl ether, dimethylaminoethyl vinyl sulfide, diethylaminoethyl vinyl ether, aminoethyl vinyl ether, 2-pyrrolidinoethyl methacrylate, 3-dimethylaminoethyl-2-hydroxy-propyl acrylate, 3-dimethylaminoethyl-2-hydroxy-propyl methacrylate, 2-aminoethyl acrylate, 2-aminoethyl methacrylate, isopropyl methacrylamide, N-methyl acrylamide, N-methyl methacrylamide, 2-hydroxyethylacrylamide, 2-hydroxyethyl methacrylamide, 1-methacryloyl-2-hydroxy-3-trimethyl ammonium chloride, 1-methacryloyl-2-hydroxy-3-trimethyl ammonium sulfomethylate, 2-(1-aziridinyl)-ethyl methacrylate, styrene, vinyl acetate, vinyl chloride, vinylidene chloride, alkyl acrylates, alkyl methacrylates, alkoxyalkyl acrylates, alkoxyalkyl methacrylates, halogenalkyl acrylates, halogenalkyl methacrylates, cyano acrylates, cyano methacrylates, acrylonitrile, vinylbenzoate, and the like.

Polymers having a very broad range of physical and chemical properties may be obtained through selection of starting monomers and the concentration of the monomer in the reaction mixture. These properties may be further varied by altering the ratio between the monofunctional and polyfunctional monomers particularly as to the solubility and swelling capacity of the resultant polymeric substances. For example, when the polymerization reaction is carried out in the presence of a radical forming catalyst utilizing the bulk polymerization technique, hard brittle polymers may be formed.

In some embodiments, one or more sanitizing layers 1010 may include polymers that include a sanitizing agent 1020. Examples of such polymers include, but are not limited to, latexes that include α,α′-azobis(chloroformamadine), polymeric vinyl halides which include copper 8-quinolinolate, acrylate or methacrylate polymers that include antimicrobial agents, polymers that include phenolic compounds, stabilized hydrophilic polymers that include a disinfectant, polymeric substances that include quaternary ammonium compounds, polyvinylpyrrolidone complexed with iodine, and polyurethane complexed with iodine. Methods to make such polymers are known (i.e., U.S. Pat. Nos.: 3,325,436; 2,689,837; 2,875,097; 2,873,263; 3,966,902; 5,142,010; 5,326,841; 5,733,270; 4,381,380; herein incorporated by reference).

In some embodiments, one or more sanitizing layers 1010 may include capsules or microspheres that include a sanitizing agent 1020 that is released during use (i.e., U.S. Pat. Nos.: 5,061,106; 5,138,719; herein incorporated by reference). Such capsules or microspheres may encapsulate one or more sanitizing agents 1020 until the capsules or microspheres are deformed or broken to release the one or more sanitizing agents 1020. For example, in some embodiments, a hydrogen peroxide producing compound may be integrally associated with one or more sanitizing layers 1010 with a catalyst that is encapsulated within a breakable capsule that releases the catalyst upon breakage of the capsule. In some embodiments, a hydrogen peroxide producing compound and a catalyst that is encapsulated within a breakable capsule may be positioned between a substantially impermeable layer 1106 and a vapor permeable layer 1102 such that breakage of the capsule provides for the release of hydrogen peroxide vapor that can permeate the vapor permeable layer 202 and sanitize the surface of the glove 1000.

Gloves and other articles may be made through use of numerous methods known in the art. For example, in some embodiments, gloves may be made through use of methods used to produce welded polyurethane films (U.S. Pat. No. 5,644,798; herein incorporated by reference). In other embodiments, gloves may be made by shaping polymeric substances through coating a support structure with a bond-preventing agent to attain a particular shape and subsequently coating the shaped structure of the bond-preventing agent with a polymeric bonding composition. Such methods are known (i.e., U.S. Pat. No. 5,501,669; herein incorporated by reference). Numerous additional methods may be used to produce gloves and other articles (i.e., U.S. Pat. Nos.: 5,549,924; 5,965,276; 6,370,694; 6,560,782; 6,913,758; 4,935,260; 4,771,482; herein incorporated by reference).

Gloves may have numerous configurations. For example, gloves may be configured as surgical gloves, examination gloves, gloves used in butchering facilities, utility gloves, gauntlet gloves, veterinary gloves, and the like. In some embodiments, gloves may cover an individual's hands. In some embodiments, gloves may cover an individual's hands and wrists. In some embodiments, gloves may cover an individual's hands and portions of their associated arm. For example, in some embodiments, gloves may cover an individual's hands and arms to the shoulder, such as with a veterinary sleeve. In other embodiments, a glove may cover an individual's hand and a portion of their associated arm, such as their wrist, forearm, elbow, upper arm, and substantially any combination thereof.

The glove 1000 may also include one or more sanitizing agents that are integrally associated with the one or more sanitizing layers and that are substantially freely releasable from the one or more substantially continually sanitizing surfaces.

Numerous types of sanitizing agents 1020 may be utilized. Sanitizing agents 1020 may include, but are not limited to, antimicrobial agents, antibiotics, antiseptics, antiviral agents, viricidal agents, bactericidal agents, antifungal agents, antiparasitic agents, and the like. In some embodiments, the terms antiseptic, disinfectant, and germicide connote a sanitizing agent 1020 which can kill microbes or pathogens upon contact. In some embodiments, the terms antiseptic, disinfectant, and germicide connote a sanitizing agent 1020 which can inactivate a microbe or pathogen upon contact.

Sanitizing agents 1020 may exist in numerous physical forms. For example, a sanitizing agent 1020 may be a solid, liquid, gas, gel, and the like. In some embodiments, a sanitizing agent 1020 may change physical form. For example, in some embodiments, a sanitizing agent 1020 may initially be in liquid form and then form a vapor. In some embodiments, a sanitizing agent 1020 may initially be in solid form that may be mixed with a liquid to form a solution, emulsion, gel, and the like. In some embodiments, a sanitizing agent 120 may exist as a solid that will melt. In some embodiments, a solid sanitizing agent 120 may melt at human body temperature. In some embodiments, a sanitizing agent 1020 may initially be in a solid form that sublimates.

In some embodiments, the sanitizing activity of a sanitizing agent 1020 may be activated, deactivated, increased, decreased, and the like. For example, in some embodiments, a sanitizing agent 1020 may be initially present in an inactive state that is activated upon contact with a fluid. In some embodiments, a sanitizing agent 1020 may be initially present in an inactive state that becomes active upon contact with the atmosphere. In some embodiments, a sanitizing agent 1020 may be initially present in an inactive state that becomes active upon contact with a catalyst. Accordingly, in some embodiments, the activity of a sanitizing agent 1020 may be controlled through regulation of conditions associated with the sanitizing agent 1020, such as concentration of sanitizing agent 1020, temperature, catalyst concentration, and the like. In some embodiments, the activity of a sanitizing agent 1020 may be decreased or deactivated. For example, in some embodiments, a quenching agent may be used to reduce or stop a chemical reaction used to generate a sanitizing agent 1020.

Examples of sanitizing agents 1020 include, but are not limited to, chlorhexidine gluconate, chlorhexidine acetate, chlorhexidine hydrochloride, chlorhexidine, other chlorhexidine salts, other hexamethylenebis biguanides, octoxynol, nonoxynol-9, methanol, ethanol, isopropanol, allyl alcohol, rubbing alcohol NF, sodium hypochlorite, potassium hypochlorite, calcium hypochlorite, magnesium hypochlorite, sodium dichloroisocyanurate, sodium perborate NF, sodium hydroxide, potassium hydroxide, magnesium hydroxide, calcium hydroxide, ammonia, ammonium hydroxide, lithium hydroxide, barium hydroxide, silver hydroxide, other metal hydroxides, sodium tetradecyl sulfate, sulfur dioxide, pentationic acid, colloidal sulfur, sulfurated potash, sublimed tyrothricin, hexachlorophene, hypochlorous acid, other chlorophors, acetic acid, hydrochloric acid, sulfuric acid, sodium acetate, aluminum acetate, acetarsone, aluminum subacetate, cadmium sulfide, selenium sulfide, other metal sulfides, bacitracin, calomel, chiniofon, creosote, diiodohydroxyquin, eucalyptol, eucalyptus oil, glycobiarsol, gramicidin, hexyl resorcinol, methylene blue, peppermint oil, phenylethyl alcohol, phenyl salicylate, methyl salicylate, pine tar, pine oil NF, pine oil emulsion, tertiary terpene alcohols, secondary terpene alcohols, alpha-terpineol, borneol, fenchyl alchol, o-methylchavicol, polymixin B sulfate, colistin, chloramphenicol, tetracycline, erythromycin, gentamycin, mafenide acetate, neomycin sulfate, sulfisoxazole diolamine, sulfacetamide sodium, gentamycin sulfate, amphotericin B, tobramycin, a penicillin, a cephalosporin, salicylic acid, trichloroacetic acid, benzoic acid, pyrogallol NF X, pyrogallic acid, sodium benzoate, boric acid, sodium borate, lactic acid, sodium lactate, chloramine, chloramine T, silver nitrate, ammoniacal silver nitrate solution, eugenol, elemental iodine, sodium iodide, potassium iodide, calcium iodide, ammonium iodide, silver iodide, colloidal silver iodide in gelatin, silver lactate, ferrous iodide, mercuric iodide red, mercuric oxide red, strontium iodide, lithium iodide, magnesium iodide, zinc iodide, silver iodide, selenium iodide, thymol iodide NF X, dithymol diiodide, iodinated derivatives of thymol, other iodide salts, povidone-iodine, iodoform, iodinated organic compounds, iodol, iodopyrrol, other iodophors, chlorinated lime, bromide salts, sodium bromide, merbromin NF, other bromophors, other brominated chemicals, sodium fluoride and other fluorinated chemicals and fluorophors, Lysol, Nonidet P40, phenyl mercuric acetate, potassium mercuric iodide, proflavine hemisulfate, 3,6-diaminoacridine bisulfate, formaldehyde, glutaraldehyde, parsformaldehyde, butyl hydroxybenzoate, mercurous chloride, iodochlorhydroxyquin, zinc nitrate, zinc sulfate, cadmium sulfate, thimerosal NF, zinc oxide, zinc acetate, zinc chloride, silver sulfadiazine, liquid peroxide, solid hydrogen peroxide complexes, peracetic acid, hydrogen peroxide, urea hydrogen peroxide, hydrogen peroxide carbamide, benzoyl peroxide, calcium peroxide, magnesium peroxide, barium peroxide, strontium peroxide, sodium peroxide, potassium perchlorite, sodium perchlorite, calcium perchlorite, magnesium perchlorite, zinc perchlorite, zinc peroxide, zinc carbonate, zinc hydroxide, zinc sulfate, succinyl peroxide, succinchlorimide NF IX, N-Chloro-succinimide, potassium permanganate, sodium chlorate, potassium chlorate, phenol, sodium phenolate, domiphen bromide, salicylic acid, bismuth-formic-iodide, bismuth subgallate, bacitracin zinc, sodium lauryl sulfate, carbamide peroxide, sodium borate, oleic acid-iodine, piperonyl butoxide, sodium peroxyborate monohydrate, ammonium ichthosulfonate, eucalyptol, menthol, Witch Hazel, camphor, tannic acid, camphorated phenol, phenol glycerin, chloroxylenol, 4-chloro-3,5-xylenol, chloroquinaldol, nalidixic acid, zinc phenol-sulfonate, zinc sulfocarbolate, hydroxynalidixic acid, pipemidic acid, norfloxacin, norfloxacin hydrochloride, other quinolones, 8-hydroxyquinoline sulfate, sodium phenolate, thyme oil, o-cresol, m-cresol, metacresylacetate, p-cresol, cresol NF, 4-chloro-m-cresol, 4-chloro-S,5-xylenol, saponified cresol solution NF, methylphenol, ethyl phenol, other alkyl phenols, o-phenyl phenol, other aryl phenols, bis-phenols, phenyl-mecuric chloride, phenylmecuric borate, resorcinol, resorcinol monoactetate NF, orthophenylphenol, chloroxylenol, hexyl-resorcinol, parachlorophenol, paratertiary-amylphenol, thymol, chlorothymol NF, menthol, butylparaban, ethylparaben, methylparaben, propylparaben, triclosan, bithionol NF, o-benzyl-p-chlorophenol, hexachlorophene, poloxamer 188, benzalkonium chloride where the alkyl groups attached to the nitrogen represent any alkyl from CH₃ to C₁₈H₃₇, methylbenzethonium chloride, cetrimonium bromide, abikoviromycin, acetylenedicarboxamide, acetyl sulfamethoxypyrazine, triclobisonium chloride, undecoylium chlorideiodine, coal tar solution, furazolidone, nifuroxime, nitrofurazone NF, nitromersol NF, oxychlorosene, sodium oxychlorosene, parachlorophenol NF, camphorated parachlorophenol NF, phenylmercuric nitrate NF, gentian violet USP, hexamethylpara-rosaniline chloride, rosaniline chloride, pentamethylpararosaniline chloride, methylrosaniline chloride, tetramethylpararosaniline chloride, nonylphenoxypolyethoxyethanol, methoxypolyoxyetheneglycol 550 laurate, oxyquinoline benzoate, p-triisopropylphenoxypolyethoxy-ethanol, halazone NF, dichloramine-T, benzethonium chloride, econazole, cetylpyridinium chloride, methylbenzethonium chloride, cetyldimethylbenzylammonium chloride, dichlorobenzalkonium chloride, domiphen bromide, triclocarban, clotrimazole, ciclopirox olamine, undecylenic acid, miconazole, tolnaftate, acriflavine, euflavine, 3,6-diamino-10-methylacridium chloride, 3,6-diamino-acridine, acid acriflavine, 5-aminoacridine hydrochloride monohydrate, malachite green G, dodecyltrimethylammonium bromide, tetradecyltrimethylammonium bromide, dequalinium chloride BP, dibromopropamidine isethionite, hexadecyltrimethylammmonium bromide, chloroazodin NF X, N-chloro-p-toluenesulfonamidosodium, 4-[(dichloroamino)sulfonyl]-benzoic acid, methenamine, methenamine mandelate, methenamine hippurate, octoxynol 9, phenazopyridine hydrochloride, 9-aminoacridine hydrochloride, bismuth tribromophenate, p-tert-butylphenol, cetyldimethylethylammonium bromide, chlorothymol, cloflucaban, clorophene, cloroxine, 8-hydroxyquinoline, merbromin, mercuric oxide yellow, ammoniated mercury, p-tert-pentylphenol, phenylmercuric acetate, phenylmercuric nitrate, propylene oxide, zinc pyrithione, zinc bacitracin, chlortetracycline hydrochloride, calcium chlortetracycline, oxytetracycline hydrochloride, beta-propiolactone, acyclovir, acyclovir sodium, amantadine hydrochloride, cytarabine, idoxuridine, interferon, gamma interferon, ribaviron, rifampin, suramin, trifluridine, vidarabine, zidovudine, methisazone, tumor necrosis factor, ampligen, ansamycin, (E)-5-(2-bromovinyl-2′-deoxyuridine, butylated hydroxytoluene, castamospermine, dextran sulfate, dideoxycytidine, dideoxyadenosine, dideoxyinosine, Peptide-T, dihydromethylpyridinylcarbonyloxyazidodideoxythymidine, ganciclovir, 2′-fluoro-2′-deoxy-5-iodo-ara C, phosphonoformate, rimantadine hydrochloride, and their derivatives and mixtures thereof. An example of a sanitizing agent 1020 that is a multicomponent mixture includes ethanol and two organic acids. In some embodiments, such a mixture includes 2% malic acid and 2% citric acid in a standard ethanol hand-sanitizing solution. This mixture has been shown to effectively inactivate rhinovirus (Smith, Interscience Conference on Antimicrobial Agents and Chemotherapy (ICAAC): Rhinovirus on Hands Blocked By Solution for Hours, Conference Report, Oct. 2, 2006, On-line report, Medpage Today, LLC, Little Falls, N.J.). Accordingly, numerous sanitizing agents 120 are known in the art and have been reported (i.e., U.S. Pat. Nos.: 5,667,753; 6,193,931; 5,357,636; herein incorporated by reference).

The glove may optionally include one or more reservoirs for the one or more sanitizing agents. In some embodiments, the one or more reservoirs 1030 may be integrally associated with a sanitizing layer 1010. For example, in some embodiments, one or more reservoirs 1030 may be contained within a sanitizing layer 1010. In some embodiments, such reservoirs 1030 may be continuously connected to one or more sanitizing surfaces such that one or more sanitizing agents 1020 may travel directly from a reservoir 1030 to the sanitizing surface 1012. In some embodiments, one or more reservoirs 1030 may be remotely connected to one or more sanitizing surfaces 1012 such that one or more sanitizing agents 1020 may travel from a reservoir 1030 through a connector, such as tubing, to the sanitizing surface. In some embodiments, a reservoir 1030 may include a device that can propel one or more sanitizing agents 1020 from a reservoir 1030 to a sanitizing surface 1012. In some embodiments, such a device may be a pump. In some embodiments, such a device may be a thermally activated compression device. For example, a compression device may include a hydrocarbon having a high vapor pressure at body temperature that is contained within a tube having a plunger such that heating the hydrocarbon to body temperature causes the plunger to move and propel a sanitizing agent 1020. In some embodiments, devices that propel sanitizing agents 1020 may be associated with, or included within, one or more operating units 1040.

The glove may optionally include one or more operating units associated with the one or more reservoirs for the one or more sanitizing agents. In some embodiments, one or more operating units 1040 may be associated with one or more reservoirs 1030. In some embodiments, an operating unit 1040 may propel one or more sanitizing agents 1020 from a reservoir 1030 to a sanitizing surface. In some embodiments, an operating unit 1040 may act to control the action of a device that propels one or more sanitizing agents 1020 from a reservoir 1030 to a sanitizing surface. In some embodiments, an operating unit 1040 may provide one or more user interfaces 1050. For example, an operating unit 1040 may include a user interface 1050 that provides for direct control of the operating unit 1040. Examples of such user interfaces 1050 include, but are not limited to, switches, buttons, levels, keyboards, touchpads, and the like. In some embodiments, an operating unit 1040 may include a user interface 1050 that provides for remote control of the operating unit 1040. Examples of such user interfaces 1050 include, but are not limited to, wireless connections, internet connections, digital signals, analog signals, optical signals, and the like.

In some embodiments, the one or more operating units provide one or more user interfaces. In some embodiments, one or more users may interact with one or more operating units 1040 through one or more user interfaces 1050. Such user interaction can include, but is not limited to, controlling an operating unit 1040 with regard to parameters associated with the operating unit 1040, a sanitizing layer 1010, and/or a sanitizing agent 1020. Parameters may include, but are not limited to, the time, place, duration, intensity, priority, and/or identity of one or more sanitizing agents 1020 that are used to sanitize a sanitizing layer 1010.

User interaction may occur directly or indirectly. For example, in some embodiments, a user may directly interact with an operating unit 1040 through one or more user interfaces 1050 that include, but are not limited to, switches, levers, buttons, a keyboard, a touchpad, and the like. In some embodiments, a user may interact with an operating unit 1040 indirectly by transmitting one or more signals from one or more user interfaces 1050 that are received by an operating unit 1040 that control transport of a sanitizing agent 1020 to a sanitizing layer 1010. In some embodiments, a user is human. In some embodiments, a user is not human.

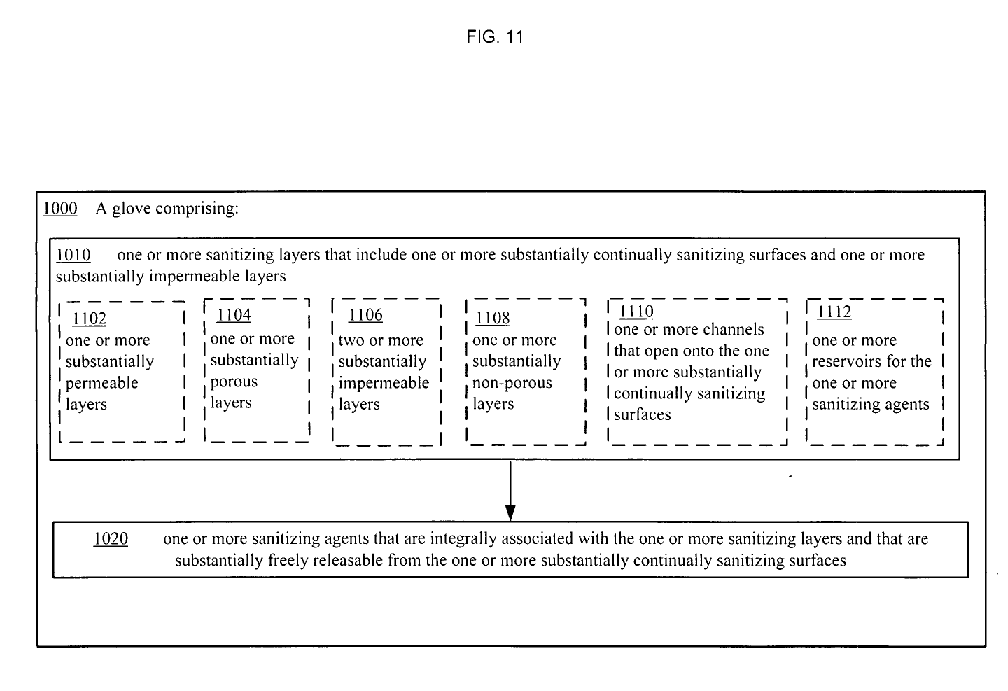

FIG. 11 illustrates alternative embodiments of the glove 1000 of FIG. 10. FIG. 11 illustrates example embodiments of the one or more sanitizing layers that include one or more substantially continually sanitizing surfaces and one or more substantially impermeable layers 1010. Additional embodiments may include embodiment 1102, embodiment 1104, embodiment 1106, embodiment 1108, embodiment 1110, and/or embodiment 1112.

At embodiment 1102, the one or more sanitizing layers that include one or more substantially continually sanitizing surfaces and one or more substantially impermeable layers may include one or more substantially permeable layers.

In some embodiments, a substantially permeable layer 1102 may include channel networks that pass through a layer. In some embodiments, the channel networks are arranged in a discontinuous manner. In some embodiments, the channel networks are arranged in a continuous manner.

For example, in some embodiments, a substantially permeable layer 1102 is loosely woven to allow passage of molecules through the layer. In some embodiments, one or more sanitizing layers 1010 may include one or more substantially permeable layers 1102 that are permeable in a phase dependent manner. For example, in some embodiments, the one or more substantially permeable layers 1102 may be permeable to liquids. For example, in some embodiments, the one or more substantially permeable layers 1102 may be permeable to gases. In some embodiments, the one or more substantially permeable layers 1102 may be permeable to gases and vapors while being impermeable to liquids. In some embodiments, the one or more substantially permeable layers 1102 may be permeable to gases and impermeable to solids. In some embodiments, the one or more substantially permeable layers 1102 may be permeable to gases and liquids and impermeable to solids. In some embodiments, the one or more substantially permeable layers 1102 may be vapor permeable and impermeable to air and moisture. In some embodiments, the one or more substantially permeable layers 1102 may be permeable in a molecular weight dependent manner. For example, in some embodiments, the one or more substantially permeable layers 1102 may be permeable to molecules of low molecular weight and impermeable to molecules of high molecular weight. In some embodiments, the one or more substantially permeable layers 1102 may be permeable to molecules in a charge dependent manner. For example, in some embodiments, the one or more substantially permeable layers 1102 may be permeable to charged molecules but impermeable to uncharged molecules. In some embodiments, the one or more substantially permeable layers 1102 may be permeable to uncharged molecules but impermeable to charged molecules. In some embodiments, the one or more substantially permeable layers 1102 may be permeable to molecules in a polarity dependent manner. For example, in some embodiments, the one or more substantially permeable layers 1102 may be permeable to polar molecules but impermeable to non-polar molecules. In some embodiments, the one or more substantially permeable layers 1102 may be permeable to non-polar molecules but impermeable to polar molecules.

Numerous substances may be used to construct one or more sanitizing layers 1010. Substances that may be used to construct substantially permeable layers 1102 can be prepared through use of numerous methods. For example, methods to prepare substances that are permeable to gas and vapor while being impermeable to fluids are known (i.e., U.S. Pat. Nos.: 4,194,041; 4,443,511; 4,692,369; 4,925,732; 5,102,711; 5,948,707; 6,521,552; 5,783,290; all of which are hereby incorporated by reference). Methods to prepare substances that are vapor permeable and impermeable to air and moisture are known (i.e., U.S. Pat. No.: 6,901,712; hereby incorporated by reference). Methods to prepare substances that are selectively gas permeable are known (i.e., U.S. Pat. No.: 6,663,805; hereby incorporated by reference). In some embodiments, apertured thermoplastic films may be used to construct permeable layers 1102. In some embodiments, apertured thermoplastic films that are treated with a surfactant may be used to construct permeable layers 1102. Such apertured thermoplastic films are known (i.e., U.S. Patent Statutory Invention Registration No.: H1,670; herein incorporated by reference). Methods to prepare liquid permeable substances are known (i.e., U.S. Pat. No.: 5,851,551; herein incorporated by reference). In some embodiments, a substantially permeable layer 1102 may include one or more apertured polymeric film webs that are liquid permeable (i.e., U.S. Published Patent Application No.: 20050214506; herein incorporated by reference). Methods to prepare substances that are selectively permeable based on molecular weight are known (i.e., U.S. Pat. No.: 5,428,123; herein incorporated by reference).

Accordingly, sanitizing layers 1010 may be designed that transport one or more sanitizing agents 1020 to a substantially continually sanitizing surface 1012 of the sanitizing layer 1010 through capillary action. Methods to prepare layers that can transport fluids through capillary action are known (i.e., U.S. Published Patent Application No.: 20050214506; herein incorporated by reference). In some embodiments, such methods may be used to prepare fluid permeable webs that may transport one or more sanitizing agents 1020 through capillary action.

In some embodiments, sanitizing layers 1010 may include two or more layers that each have physical properties that are different from each other. For example, in some embodiments, a hydrophobic layer and a hydrophilic layer may be oriented relative to each other to promote and/or inhibit the movement of hydrophobic and/or hydrophilic substances relative to the hydrophobic and/or hydrophilic layers. Such constructions of layers may be used to promote and/or inhibit wicking of substances, such as sanitizing agents 1020, through the sanitizing layer 1010. In some embodiments, such constructions of layers may be used to direct the flow of substances relative to the sanitizing layer 1010.

In some embodiments, permeable substances may be prepared by polymerizing monomers into polymers. Examples of monomers that may be used to prepare substantially permeable layers 1102 are described herein and are known in the art.

In some embodiments, cross-linking agents may be used to produce polymeric layers having various degrees of permeability. Numerous cross-linking agents may be used that include, but are not limited to, diesters of acrylic acid, diesters of methacrylic acid, ethyleneglycol diacrylate, ethyleneglycol dimethacrylate, 1,2-butyleneglycol diacrylate, 1,2-butyleneglycol dimethacrylate, 1,3-butyleneglycol diacrylate, 1,3-butyleneglycol dimethacrylate, 1,4-butyleneglycol diacrylate, 1,4-butyleneglycol dimethacrylate, propyleneglycol diacrylate, propyleneglycol dimethacrylate, diethyleneglycol diacrylate, diethyleneglycol dimethacrylate, dipropyleneglycol diacrylate, dipropyleneglycol dimethacrylate, divinyl benzene, divinyl toluene, diallyl tartrate, allyl pyruvate, allyl maleate, divinyl tartrate, triallyl melamine, N,N′-methylene bis acrylamide, glycerine dimethacrylate, glycerine trimethacrylate, diallyl maleate, divinyl ether, diallyl monoethyleneglycol citrate, ethyleneglycol vinyl allyl citrate, allyl vinyl maleate, diallyl itaconate, ethyleneglycol diester of itaconic acid, divinyl sulfone, hexahydro 1,3,5-triacyltriazine, triallyl phosphite, diallyl ether of benzene phosphonic acid, maleic anhydride triethylene glycol polyester, polyallyl sucrose, polyallyl glucose, sucrose diacrylate, glucose dimethacrylate, pentaerythritol di-, tri- & -tetraacrylate or methacrylate, trimethylol propane di- and triacrylate or methacrylate, sorbitol dimethacrylate, 2-(1-aziridinyl)-ethyl methacrylate, tri-ethanolamine diacrylate or dimethacrylate, triethanolamine triacrylate or trimethacrylate, tartaric acid dimethacrylate, triethyleneglycol dimethacrylate, the dimethacrylate of bis-hydroxy ethylacetamide and the like.

If the polymerization reaction is conducted in the presence of a solvent, soluble linear or branched chain complex polymers and copolymers may be obtained. In some embodiments, conducting the polymerization reaction in the substantial absence of any solvent produces products that constitute rigid macroporous polymer substances. In some embodiments, polymerization may be carried out in the presence of a solvent which is effective for only partially swelling the polymer such that soft sponge-like polymer products are obtained. Accordingly, the degree of cross-linking which takes place in the polymerization reaction also influences the properties of the resultant polymeric products.

Examples of solvents include, but are not limited to, hydrophilic substances such as water, alcohol, ketone, glycol, glycol ester, glycol ether, amide, alkyl amide, and the like. In some embodiments, a hydrophilic solvent can be replaced by an appropriate hydrophobic solvent, such as an aromatic, aliphatic or halogenated hydrocarbon, ether, ester, or the like.

The polymerization reactions may be initiated in the conventional manner through use of radical forming initiators. Examples of such initiators include, but are not limited to, dibenzoyl peroxide, tert. butyl peroctoate, cumene hydroperoxide, diazodilsobutyrodinitrille, diisopropylpercarbonate, ammonium persulfate, and the like, alone or in combination with a reducing agent.

In some embodiments, one or more permeable sheets may be laminated together to form a substantially permeable layer 1102 of one or more sanitizing layers 1010. In some embodiments, one or more permeable sheets may be laminated together to form a selectively permeable layer 1102. For example, in some embodiments, a sheet that is impermeable to one or more charged chemical compounds may be laminated onto a sheet that is impermeable to one or more chemical compounds that are above a given mass (i.e., 150 grams/mole). Accordingly, such a semi-permeable layer will be permeable to uncharged chemical compounds having a molecular mass that is below the given mass (i.e., 150 grams/mole). Numerous combinations of sheets may be used to control the permeability of one or more sanitizing layers 1010. Methods to prepare multi-layer sheets are known and have been described (i.e., U.S. Pat. No.: 5,648,003; herein incorporated by reference).

In some embodiments, one or more sanitizing layers 1010 may include one or more substantially permeable substances that can control the release of one or more sanitizing agents 1020 from the one or more sanitizing layers 1010. In some embodiments, one or more sanitizing layers 1010 may include one or more substantially permeable substances that can control the release of one or more sanitizing agents 1020 from one or more substantially continually sanitizing surfaces 1012 of the one or more sanitizing layers 1010.

In some embodiments, one or more sanitizing layers 1010 may include one or more substantially permeable layers 1102 that provide for the release of one or more sanitizing agents 1020 in vapor and/or gas form from one or more sanitizing layers 1010. In some embodiments, one or more sanitizing layers 1010 may include one or more substantially permeable substances that may control the release of one or more sanitizing agents 1020 in vapor and/or gas form from one or more substantially continually sanitizing surfaces 1012. For example, in some embodiments, one or more substantially permeable layers 1102 may provide for release of peroxide vapor. In some embodiments, the peroxide vapor may be released from one or more surfaces of the one or more sanitizing layers 1010. In some embodiments, the peroxide vapor may be released from one or more substantially continually sanitizing surfaces 1012 of the one or more sanitizing layers 1010. In some embodiments, the peroxide vapor may be generated through use of one or more inorganic hydrogen peroxide complexes. Numerous inorganic hydrogen peroxide complexes exist and include, but are not limited to, alkalimetal carbonates, ammonium carbonates, alkali metal oxalates, alkali metal phosphates, alkali metal pyrophosphates fluorides, hydroxides, sodium carbonate hydrogen peroxide complexes, complexes of hydrogen peroxide with polymeric N-vinylheterocyclic compounds, complexes of hydrogen peroxide and solid polymeric electrolytics. Such complexes are known and have been described (i.e., U.S. Pat. Nos.: 2,986,448; 3,870,783; 3,376,110; 3,480,557; 5,008,093; 5,077,047; 5,030,380; herein incorporated by reference).

In some embodiments, one or more sanitizing layers 1010 may include one or more permeable layers 1102 that provide for the release of one or more sanitizing agents 1020 in liquid and/or gel form from one or more sanitizing layers 1010. In some embodiments, one or more sanitizing layers 1010 may include one or more permeable substances that may control the release of one or more sanitizing agents 1020 in liquid and/or gel form from one or more substantially continually sanitizing surfaces 1012. Numerous sanitizing agents 1020 may be prepared as liquids and/or gels for release from one or more sanitizing layers 1010. Examples of such sanitizing agents 1020 that may be prepared as liquids and/or gels include, but are not limited to, alkalies/hydroxides (i.e., sodium hydroxide, caustic soda, soda lye, calcium oxide (lime)), biguanides/chlorhexidine (i.e., volvasan®, virosan, chlorhexiderm), cationic surfactants/quaternary ammonium compounds (i.e., parvosol™, roccal-D® plus, A33™, maxima 128, ken-care, unicide 256, benzalkonium chloride, bensathonium chloride, cetylpyridinium chloride), halogens and halogen-containing compounds (i.e., sodium hypochlorite (chlorine bleach), alcide, sodium dichloroisocyanurate, calcium hypochlorite), iodine-based (iodine, iodophors, povidone-iodine, betadine), oxidizing agents/peroxides (i.e., ozone, hydrogen peroxide, sodium perborate, benzoyl peroxide, potassium permanganate), peroxygen compounds (i.e., stabilized chlorine dioxide), phenols and related compounds/phenolics (i.e., phenol (carbolic acid), cresol (cresylic acid), lysol, pine tar, pine oil), synthetic phenol (i.e., chloroxylenols, hexachlorophene, sporicidin, parachlorometaxylenol (PCMX), dichlorometaxylenol (DCMX)), reducing agents/aldehydes (i.e., glutaral (glutaraldehyde), formalin (formaldehyde)), and alcohols. Such sanitizing agents 1020 are known (i.e., U.S. Pat. Nos.: 4,642,165; 4,744,951; 5,008,106; herein incorporated by reference).

In some embodiments, the permeability of a substantially permeable layer 1102 may be dependent upon stretching or deformation of the substantially permeable layer. For example, in some embodiments, the permeability of a substantially permeable layer 1102 in a compressed form may be low but may be high when the same layer is stretched to deform the layer and thereby increase the permeability of the layer.

At embodiment 1104, the one or more sanitizing layers that include one or more substantially continually sanitizing surfaces and one or more substantially impermeable layers may include one or more substantially porous layers.

In some embodiments, a substantially porous layer 1104 includes passages that pass through a layer in a continuous manner. Numerous types of substances may be used to prepare sanitizing layers 1010 that include one or more substantially porous layers 1104. For example, porous plastics may be used to prepare such substantially porous layers 204. Examples of such porous plastics include, but are not limited to, thermoplastic polymers, such as polyethylene, polypropylene, polyvinylferrocene, nylon, polyether sulphone, expanded polytetrafluoroethylene films, and the like. Substantially porous layers 1104 may be prepared through use of methods as described herein and as are known in the art (i.e., U.S. Pat. Nos.: 6,676,871; 3,953,566; 6,252,128; 4,187,390; 6,765,029; 5,798,165; 5,779,795; and 5,641,566; herein incorporated by reference). In some embodiments, a substantially porous layer 1104 may be metallic (i.e., U.S. Pat. No.: 5,641,566; herein incorporated by reference).

In some embodiments, a substantially porous layer 1104 may be prepared from one or more substances such as that sold by Porex Technologies (Fairburn, Ga., Porex®). Porex® is a microporous sheet with known pore size. Porex® is permeable to vapor, but not aqueous liquids (i.e., it is hydrophobic). In some embodiments, the Porex® may be used to provide for constant delivery and release of a sanitizing agent 1020 from the surface of a sanitizing layer 1010 (i.e., U.S. Pat. No.: 5,733,270; herein incorporated by reference). Additionally, Porex® is thought to be impervious to microorganisms.

In some embodiments, pores may be created in a substantially porous layer 1104 through physical methods (i.e., U.S. Pat. No. 5,269,981; herein incorporated by reference). Briefly, a sheet may be placed on a pattern anvil having a pattern of raised areas wherein the height of the raised areas is greater than the thickness of the sheet; the sheet is conveyed, while placed on the pattern anvil, through an area where a fluid is applied to the sheet; and the sheet is subjected to a sufficient amount of ultrasonic vibration in the area where the fluid is applied to the sheet to microaperture the sheet in a pattern, generally the same as the pattern of raised areas on the pattern anvil.

In some embodiments, the density and structure of the pores associated with a sanitizing layer 1010 control, at least in part, delivery of a sanitizing agent 1020 to the surface of a sanitizing surface and release of the sanitizing agent 1020 from the surface of a sanitizing layer 1010. Through selection of pore structure, size of the area for permeation, control of length and height, and selection of material, the delivery of a sanitizing agent 1020 and release of the sanitizing agent 1020 from the surface of a sanitizing surface may be controlled.

At embodiment 1106, the one or more sanitizing layers that include one or more substantially continually sanitizing surfaces and one or more substantially impermeable layers may include two or more substantially impermeable layers.

Substantially impermeable layers 1106 can include layers that are impermeable to liquids, gases, and solids. Substantially impermeable layers 1106 may have numerous configurations and be made from numerous types of substances.

In some embodiments, substantially impermeable layers 1106 may include water-impermeable and gas-impermeable substances. Methods to prepare water-impermeable sheets and essentially gas-impermeable thermoplastic elastomers are known and have been described (U.S. Pat. Nos.: 4,312,907 and 7,056,971; herein incorporated by reference).

In some embodiments, the substantially impermeable layer 1106 may include a thermoplastic polymer. An example of such a thermoplastic polymer includes, but is not limited to, high-density polyethylene. In some embodiments, at least some of the high-density polyethylene may be replaced by other polymers that may include, but are not limited to, polypropylene and its copolymers, such as polypropylene/polyethylene, and terpolymers, such as poly-(propylene-butene/ethylene). In some embodiments, poly-(ethylene-vinylalcohol) may be mixed with high-density polyethylene (see U.S. Pat. No.: 5,731,053; herein incorporated by reference). In some embodiments, a substantially impermeable layer 1106 may include one or more styrene-ethylene-butylene-styrene copolymers, one or more styrene-butadiene-styrene copolymers, and/or one or more styrene-isoprene-styrene copolymers (see U.S. Pat. No.: 5,480,915; herein incorporated by reference).

In some embodiments, one or more impermeable layers 1106 may include one or more elastic sheets that include a substantially continuous ductile metal coating that is able to repeatedly expand and contract with the elastic sheet. Such metal coated elastic sheets are able to expand and contract without fracturing or breaking the metal layer. Methods to prepare such metal coated elastic sheets are known (i.e., U.S. Pat. Nos. 5,069,227; 5,113,874; herein incorporated by reference).

At embodiment 1108, the one or more sanitizing layers that include one or more substantially continually sanitizing surfaces and one or more substantially impermeable layers may include one or more substantially non-porous layers.

In some embodiments, non-porous substances may be prepared from one or more substances selected from modified polysiloxane based elastomers that include polydimethylsiloxane based elastomers, ethylene-propylene diene based elastomers, polynorbornene based elastomers, polyoctenamer based elastomers, polyurethane based elastomers, butadiene and nitrile butadiene rubber based elastomers, natural rubber, butyl rubber based elastomers, polychloroprene based elastomers, epichlorohydrin elastomers, polyacrylate elastomers, polyethylene, polypropylene, polytetrafluoroethylene, polyvinylidene difluoride based elastomers, and mixtures thereof (i.e., U.S. Pat. No.: 6,716,352; herein incorporated by reference). For example, in some embodiments, a non-porous layer may be prepared from a non-porous polyurethane film block copolymer. Such polyurethane film block copolymers may be prepared through reaction of a diisocyanate and a polyethylene glycol that is present in the amount of from 25 to 45% by weight based on the total weight of the reaction mixture, to produce the non-porous polyurethane film block copolymer.

In some embodiments, a substantially non-porous layer may include a fluid impermeable foil. Such foils are known and may be prepared by vapor depositing a metallic film onto a substrate (i.e., U.S. Pat. No. 6,524,698; herein incorporated by reference). Numerous methods that may be used to prepare non-porous substances are known and have been described.

At embodiment 1110, the one or more sanitizing layers that include one or more substantially continually sanitizing surfaces and one or more substantially impermeable layers may include one or more channels that open onto the one or more substantially continually sanitizing surfaces.

In some embodiments, one or more substantially continually sanitizing surfaces 1012 may include one or more channels 1110 on their surfaces. In some embodiments, one or more substantially continually sanitizing surfaces 1012 may include one or more channels 1110 that are contained within the one or more sanitizing layers 1010 and that open onto the one or more sanitizing surfaces 1012. In some embodiments, one or more sanitizing agents 1020 may travel through the one or more channels 1110 through capillary action. In some embodiments, the one or more channels 1110 may be continuously connected to one or more reservoirs 1030 that may contain one or more sanitizing agents 1020.

In some embodiments, one or more sanitizing agents 1020 may be propelled through one or more channels 1110 through the use of a propellant. In some embodiments, one or more sanitizing agents 1020 may be contained within a reservoir 1030 that is connected to the one or more channels 1110 such that a sanitizing agent 1020 may be propelled from the reservoir 1030 through the channels 1110 onto a sanitizing surface through use of a propellant. Numerous compounds may be used as propellants. For example, in some embodiments, low molecular weight hydrocarbons, such as alkanes, alkynes, alkenes, alcohols, ethers, esters, and the like, may be used as propellants. In some embodiments, such hydrocarbons may exist as liquids at room temperature that exhibit a high vapor pressure. Accordingly, such fluid hydrocarbons may be caused to vaporize and thereby propel sanitizing agents 1020 through one or more channels 1110. In some embodiments, hydrocarbons may be selected that vaporize at human body temperature, such as pentane, to propel a sanitizing agent 1020 through one or more channels 1110 upon contact of a glove 1000 containing the one or more channels 210 with a human. Numerous propellants may be used to propel a sanitizing agent 1020 through one or more channels 1110.

In some embodiments, one or more pumps may be connected to one or more channels 1110 such that action of the one or more pumps will propel one or more sanitizing agents 1020 through the one or more channels 1110. In some embodiments, one or more of the pumps may be controlled through use of an operating unit 1040.

Release of one or more sanitizing agents 1020 from a channel 1110 can be controlled through controlling the size of the channel 1110 and the pressure exerted on the channel 1110. Accordingly, channel 1110 characteristics may be selected to control the release of one or more sanitizing agents 1020 from a substantially continually sanitizing surface 1012.

At embodiment 1112, the one or more sanitizing layers that include one or more substantially continually sanitizing surfaces and one or more substantially impermeable layers may include one or more reservoirs for the one or more sanitizing agents.

In some embodiments, a sanitizing layer 1010 may include one or more reservoirs 1030. In some embodiments, such reservoirs 1030 may contain one type of sanitizing agent 1020. In some embodiments, such reservoirs 1030 may contain one or more types of sanitizing agents 1020. In some embodiments, such reservoirs 1030 may contain components other than sanitizing agents 1020. For example, such reservoirs 1030 may contain coloring agents, olfactory agents, and the like.

In some embodiments, reservoirs 1030 may be integrally associated with a sanitizing layer 1010. For example, in some embodiments, a reservoir 1030 may be contained within a sanitizing layer 1010 that opens onto the surface of the sanitizing layer 1010. In other embodiments, a sanitizing layer 1010 may include a reservoir 1030 that connects to a channel 210 that opens onto the surface of the sanitizing layer 1010. Accordingly, reservoirs 1030 may be configured in numerous geometries.

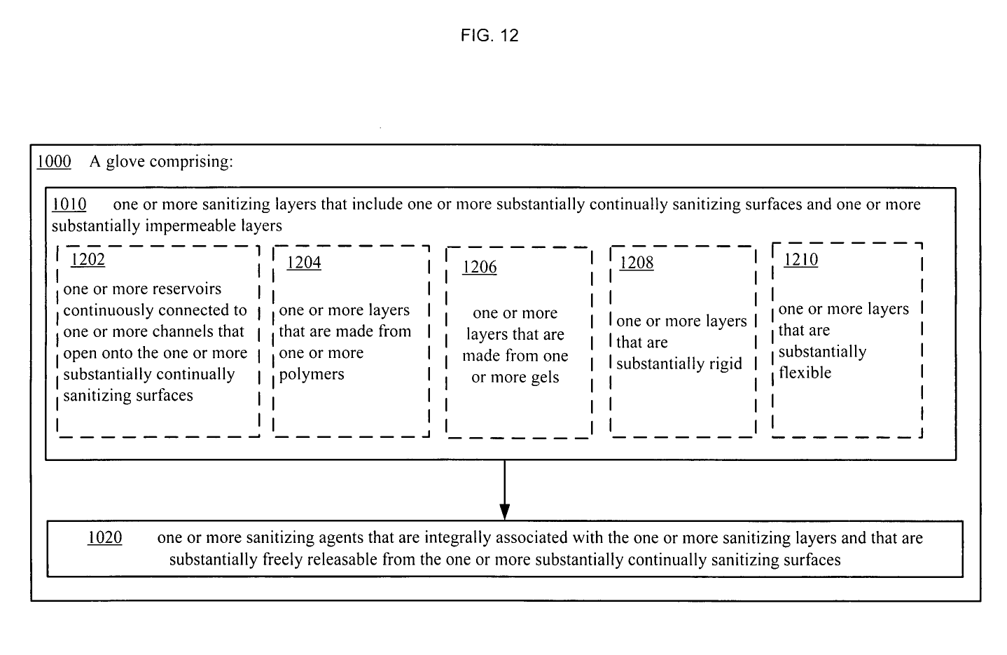

FIG. 12 illustrates alternative embodiments of the glove 1000 of FIG. 10. FIG. 12 illustrates example embodiments of the one or more sanitizing layers that include one or more substantially continually sanitizing surfaces and one or more substantially impermeable layers. Additional embodiments may include embodiment 1202, embodiment 1204, embodiment 1206, embodiment 1208, and/or embodiment 1210.

At embodiment 1202, the one or more sanitizing layers that include one or more substantially continually sanitizing surfaces and one or more substantially impermeable layers may include one or more reservoirs continuously connected to one or more channels that open onto the one or more substantially continually sanitizing surfaces.

Reservoirs 1030 may be configured in numerous geometries. In some embodiments, the one or more reservoirs 1030 may be contained within one or more sanitizing layers 1010. In some embodiments, the one or more reservoirs 1030 may be separate from the one or more sanitizing layers 1010 and connected to the one or more sanitizing layers 1010 through use of a connector, such as tubing. For example, in some embodiments, a reservoir 1030 may be a tank that is connected to a sanitizing layer 1010 through the use of tubing that allows a sanitizing agent 1020 to be transported from the reservoir 1030 to the sanitizing layer 1010. In some embodiments, a pump, compressor, or other device may be associated with the reservoir 1030 to propel a sanitizing agent 1020 to an associated sanitizing layer 1010. Such devices may be associated with an operating unit 1040 to provide for controlled delivery of a sanitizing agent 1020 to a sanitizing layer 1010. For example, such devices may include circuitry that can be used to control operation of the device. In some embodiments, the circuitry may be used to control delivery of a sanitizing agent 1020 from a reservoir 1030 to a sanitizing surface upon receipt of a signal.

At embodiment 1204, the one or more sanitizing layers that include one or more substantially continually sanitizing surfaces and one or more substantially impermeable layers may include one or more layers that are made from one or more polymers.

Numerous polymers are known and have been described herein that may be used to prepare one or more sanitizing layers 1010. Sanitizing layers 1010 that include polymers may exhibit numerous properties. Examples of such properties include, but are not limited to, hardness, softness, malleability, pliability, flexibility, stiffness, permeability, impermeability, porosity, non-porosity, elasticity, chemical imperviousness, thermal conductivity, insulation ability, conductivity, hydrophobicity, hydrophilicity, electrical conductivity, self-siphoning ability, and the like.

At embodiment 1206, the one or more sanitizing layers that include one or more substantially continually sanitizing surfaces and one or more substantially impermeable layers may include one or more layers that are made from one or more gels.

In some embodiments, one or more sanitizing layers 1010 may include gelatinous elastomer compositions that include a selectively hydrogenated triblock copolymer of styrene and butadiene and an excess by weight of a plasticizing oil (i.e., U.S. Pat. No.: 4,618,213; herein incorporated by reference). In some embodiments, one or more sanitizing layers 1010 may include a hydrocarbon-oil and a mixture of two selectively hydrogenated styrene/butadiene triblock polymers of particular composition in a particular weight ratio (i.e., U.S. Pat. No.: 4,716,183; herein incorporated by reference). In some embodiments, styrene-diene block copolymers may be used. A styrene-diene block copolymer is a poly (styrene-ethylene-butylene-styrene) triblock copolymer (SEBS triblock copolymers) which, when combined with sufficient plasticizer, such as a hydrocarbon oil, provides a gel composition. For example, these triblock copolymers may be melt blended with oils to produce a gel-like polymer which is meltable and useful for cast molding of shaped articles. Numerous gels and methods to produce them are known and have been reported (i.e., U.S. Pat. Nos.: 4,942,270; 4,369,284; 4,618,213; 3,827,999; 4,176,240; 3,485,787; 3,376,384; 4,716,183; 4,556,464; herein incorporated by reference).

In some embodiments, the pore size and/or the permeability of porous and/or permeable gels may be selected to control release of one or more sanitizing agents 1020 from a sanitizing surface 1012. For example, in some embodiments, a sanitizing layer 1010 may include a gel having very large pores that provides for greater release of one or more sanitizing agents 1020 relative to a gel having smaller pores. Accordingly, sanitizing agent 1020 release may be regulated through control of pore size.

In some embodiments, gels may be selected in which the pore size may be dynamically controlled. For example, pores may be opened or closed following a select event or condition. For example, in some embodiments, gels may be selected in which the pore size and/or permeability is temperature dependent. In other embodiments, a gel may be selected in which the pore size and/or permeability of the gel is dependent upon electrical current flowing through the gel. Accordingly, the release of one or more sanitizing agents 1020 from a sanitizing layer 1010 that includes such a gel may be regulated by controlling the amount of electrical current applied to the gel. In some embodiments, gels may be selected in which the pore size and/or permeability is dependent upon exposure to one or more chemicals. Accordingly, the release of one or more sanitizing agents 1020 from a sanitizing layer 1010 that includes such a gel may be regulated by controlling the exposure of one or more chemicals to the gel.

At embodiment 1208, the one or more sanitizing layers that include one or more substantially continually sanitizing surfaces and one or more substantially impermeable layers may include one or more layers that are substantially rigid.

Gloves 1000 may include layers that are substantially rigid 1208. In some embodiments, substantially rigid layers 1208 may be articulated such that they are able to flex with movement of a glove 1000. For example, in some embodiments, substantially rigid layers 1208 may be flexibly connected to each other. Accordingly, these substantially rigid layers 1208 may correspond in length to the length of the metacarpals of the hand such that the position of flexible connectors holding the substantially rigid layers 1208 together corresponds to the position of the joints of the hand when the glove 1000 is worn. Such an orientation provides for substantially unrestricted hand movement when gloves 1000 are worn that include substantially rigid layers 1208.

Generally, layers that are substantially rigid include those that are substantially resistant to being bent, flexed, and/or stretched. However, designation of a layer as being substantially rigid does not mean that the layer is entirely unable to bend, flex, and/or stretch. For example, a substantially rigid layer 1208 may be constructed from stainless steel such that it is substantially resistant to being bent but still exhibits a moderate ability to flex. Examples of other substances that may be used to construct substantially rigid layers 1208 include, but are not limited to, metal, plastic, hard rubber, and the like.

At embodiment 1210, the one or more sanitizing layers that include one or more substantially continually sanitizing surfaces and one or more substantially impermeable layers may include one or more layers that are substantially flexible.

Generally, layers that are substantially flexible 1210 include those that can be substantially bent, flexed, and/or stretched. However, designation of a layer as being substantially flexible does not mean that the layer lacks structural rigidity. For example, a substantially flexible layer 1210 may be constructed from latex rubber such that it may be significantly bent, flexed, and/or stretched while being able to return to its initial shape. Examples of other substances that may be used to construct substantially flexible layers include, but are not limited to, metal, plastic, ceramic, rubber, glass, and the like. In some embodiments, a single type of substance may be used to construct a substantially flexible layer while in other embodiments the substance may be used to construct a substantially rigid layer. For example, in some embodiments, glass may be used to construct a substantially rigid layer 1208 while in other embodiments, glass fibers may be used to construct a substantially flexible layer 1210.

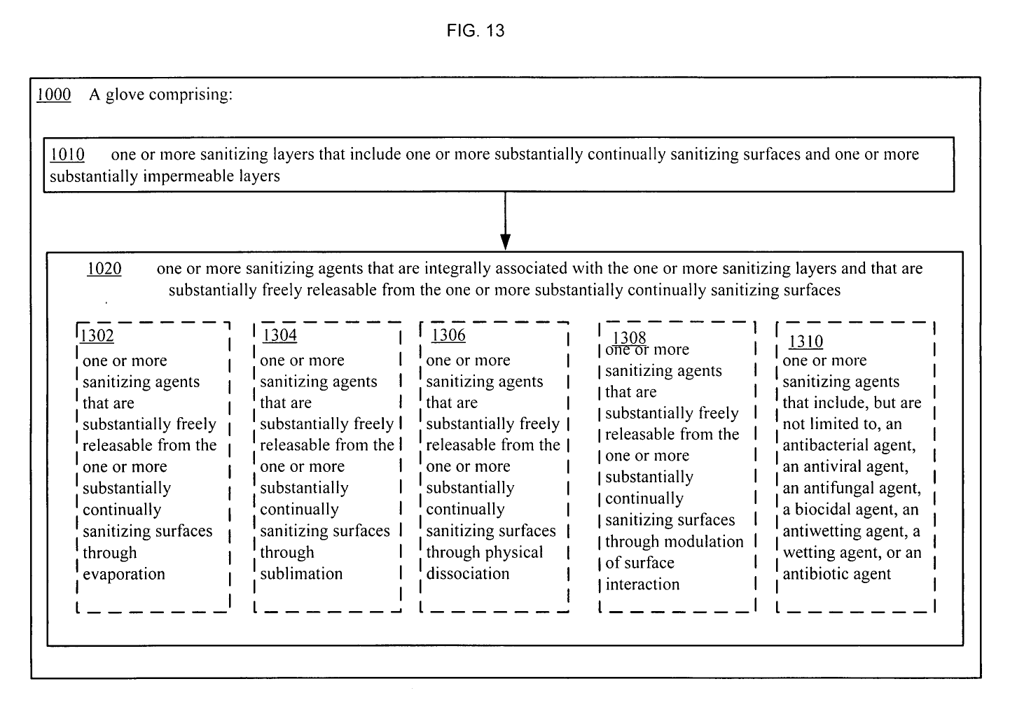

FIG. 13 illustrates alternative embodiments of the glove 1000 of FIG. 10. FIG. 13 illustrates example embodiments of the one or more sanitizing agents that are integrally associated with the one or more sanitizing layers and that are substantially freely releasable from the one or more substantially continually sanitizing surfaces 1020. Additional embodiments may include embodiment 1302, embodiment 1304, embodiment 1306, embodiment 1308, and/or embodiment 1310.

At embodiment 1302, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through evaporation.

Sanitizing agents 1020 that are able to transition from the liquid phase to the gaseous phase may be used in association with a sanitizing layer 1010. In some embodiments, sanitizing agents 1020 having adequate vapor pressures to cause the sanitizing agents 1020 to evaporate at room temperature are provided. In some embodiments, sanitizing agents 1020 having adequate vapor pressures to cause the sanitizing agent 1020 to evaporate at human body temperature are provided. Numerous such sanitizing agents 1020 are known and have been described herein.

In some embodiments, release of a sanitizing agent 1020 from a substantially continually sanitizing surface 1012 through evaporation may be controlled by the characteristics of the sanitizing layer 1010, such as the density of the layer, the thickness of the layer, the characteristics of pores through the layer, the permeability of the layer, and the like. For example, the rate of release of one or more sanitizing agents 1020 may be controlled by the size and shape of pores associated with the sanitizing layer 1010, the thickness of the sanitizing layer 1010, the temperature of the sanitizing layer 1010, and the like.

In some embodiments, release of a sanitizing agent 1020 from a substantially continually sanitizing surface 1012 through evaporation may be controlled according to diffusion of the sanitizing agent 1020 through the sanitizing layer 1010. For example, the rate of release of one or more sanitizing agents 1020 may be controlled by the permeability of the layer or layers used to prepare the sanitizing layer 1010, the thickness of the sanitizing layer 1010, the temperature of the sanitizing layer 1010, and the like.

In some embodiments, release of a sanitizing agent 1020 from a substantially continually sanitizing surface 1012 through evaporation may be controlled through formulation of the sanitizing agent 1020. For example, a sanitizing agent 1020 may be formulated with carriers having high vapor pressures at room temperature to increase release of the sanitizing agent 1020 as compared to the release of a sanitizing agent 1020 that was formulated with a carrier having low vapor pressure at room temperature. Accordingly, sanitizing agents 1020 may be formulated in numerous forms to control release of the sanitizing agent 1020 through evaporation.

At embodiment 1304, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through sublimation.

Sanitizing agents 1020 that are able to transition from the solid phase to the gaseous phase may be used in association with a sanitizing layer 1010. In some embodiments, sanitizing agents 1020 having adequate vapor pressures to cause the sanitizing agents 1020 to sublimate at room temperature are provided. In some embodiments, sanitizing agents 1020 having adequate vapor pressures to cause the sanitizing agent 1020 to sublimate at human body temperature are provided. Such sanitizing agents 1020 include, but are not limited to, halogens, halogen compounds, molecular iodine, 1,4-dichlorobenzene, and the like (i.e., U.S. Pat. No.: 5,733,270; herein incorporated by reference).

In some embodiments, release of a sanitizing agent 1020 from a substantially continually sanitizing surface 1012 through sublimation may be controlled by the characteristics of the sanitizing layer 1010, such as the density of the layer, the thickness of the layer, the characteristics of pores through the layer, the permeability of the layer, and the like. For example, the rate of release of one or more sanitizing agents 1020 may be controlled by the size and shape of pores associated with the sanitizing layer 1010, the thickness of the sanitizing layer 1010, the temperature of the sanitizing layer 1010, and the like.

In some embodiments, release of a sanitizing agent 1020 from a substantially continually sanitizing surface 1012 through sublimation may be controlled according to diffusion of the sanitizing agent 1020 through the sanitizing layer 1010. For example, the rate of release of one or more sanitizing agents 1020 may be controlled by the permeability of the layer or layers used to prepare the sanitizing layer 1010, the thickness of the sanitizing layer 110, the temperature of the sanitizing layer 1010, and the like.

At embodiment 1306, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through physical dissociation.

In some embodiments, one or more substantially continually sanitizing surfaces 1012 may be constructed such that one or more sanitizing agents 1020 may physically dissociate. For example, in some embodiments, a substantially continually sanitizing surface 1012 may be constructed with particles that are held within a dissociable matrix. As the matrix dissociates, particles that are held within the matrix will dissociate from the substantially continually sanitizing surface 1012 to expose a sanitized surface. Matrices may dissociate through numerous mechanisms that include, but are not limited to, physical degradation, sublimation, oxidation mediated degradation, light mediated degradation, and the like.

Particles that may be included within a substantially continually sanitizing surface 1012 may be of numerous shapes. In some embodiments, particles may be substantially uniform in shape. In some embodiments, particles may vary substantially in shape. For example, particles may be beads, flakes, disks, threads, rods, circles, ovals, ellipses, triangles, squares, rectangles, pentagons, hexagons, stars, barbells, and the like. In some embodiments, particle shape may be selected with regard to the substance or substances used to construct the matrix. Particle shapes may also be selected such that the rate of dissociation and/or conditions under which the particles will dissociate may be controlled.

Particles that may be included within a substantially continually sanitizing surface 1012 may be of numerous sizes. In some embodiments, particles may be substantially uniform in size. In some embodiments, particles may vary substantially in size. For example, in some embodiments, particles may be within a range of about 1 nanometer to about 1 centimeter. In some embodiments, particles may be within a range of about 1 millimeter to about 1 centimeter. In some embodiments, particles may be within a range of about 1 millimeter to about 10 millimeters. In some embodiments, particles may be within a range of about 1 millimeter to about 5 millimeters. In some embodiments, particle size may be selected with regard to the substance or substances used to construct the matrix. Particle sizes may also be selected such that the rate of dissociation and/or conditions under which the particles will dissociate may be controlled. In some embodiments, particles may be selected such that they are able to be cleared from an area through normal air circulation. For example, in some embodiments, particles may essentially float as airborne particles that can be collected within filters present in the heating and cooling network associated with the area. In some embodiments, particles may be selected that will fall to the ground following dissociation from a substantially continually sanitizing surface 1012.

Numerous substances may be used to produce particles for inclusion in a substantially continually sanitizing surface 1012. Examples of such substances include, but are not limited to, silica, plastic, metal, polymeric substances, and the like.

Numerous substances may be used to produce a matrix that will dissociate over time and release particles contained within the matrix. In some embodiments, a substance may be used to produce a matrix that will dissociate through sublimation. For example, in some embodiments, particles may be included within a 1,4-dichlorobenzene matrix that will sublimate over time and cause the particles contained within the matrix to dissociate.

In some embodiments, matrices may be produced that undergo light-mediated dissociation. For example, in some embodiments, matrices may be produced from short polymers that are cross-linked with photocleavable linkers. Accordingly, as the photocleavable cross-linkers are cleaved through the action of light, the short polymers are able to dissociate and free particles that are included within the polymeric matrix. Photocleavable substances are known and have been described (i.e., U.S. Pat. Nos.: 6,806,361; 5,563,238; 5,360,892; 4,197,375; 4,073,764; 4,042,765; and 4,476,255; hereby incorporated by reference). In some embodiments, an ultraviolet light degradable polymer may be a polylactic acid polymer that includes a copolymer of polylactic acid and a modifying monomer. Such modifying monomers include, but are not limited to, p-dioxanone, 1,5 dioxepan-2-one, and 1,4 oxathialan-2-one, 4,4 dioxide, or mixtures thereof (U.S. Pat. No.: 5,563,238; hereby incorporated by reference). In some embodiments, matrices may be produced that undergo oxidation-mediated dissociation. For example, in some embodiments, matrices may be produced from short polymers that are cross-linked with linkers that may be cleaved through atmosphere-mediated oxidation. Accordingly, oxidation-mediated cleavage of the cross-linkers retaining the short polymers may allow the polymers to dissociate and free particles that are included within the polymeric matrix.

At embodiment 1308, the one or more sanitizing agents that are integrally associated with the one or more sanitizing layers and that are substantially freely releasable from the one or more substantially continually sanitizing surfaces may include one or more sanitizing agents that are substantially freely releasable from the one or more substantially continually sanitizing surfaces through modulation of surface interaction.

In some embodiments, the interaction of a substantially continually sanitizing surface 1012 and one or more sanitizing agents 1020 may be regulated to control release of the one or more sanitizing agents 1020 from the sanitizing surface 1012. For example, in some embodiments, one or more sanitizing surfaces 1012 may be constructed such that they provide for low energy release of one or more sanitizing agents 1020. Such sanitizing surfaces 1012 may include silicone rubber, polytetrafluoroethylene, or other substances which are known to exhibit good release properties (i.e., U.S. Pat. No.: 5,779,795; herein incorporated by reference).

In some embodiments, the surface interaction of a sanitizing surface 1012 and one or more sanitizing agents 1020 may be modulated through selecting a combination of one or more sanitizing agents 1020 and sanitizing surfaces 1012 based upon the physical and chemical properties of the sanitizing agents 1020 and sanitizing surfaces 1012. For example, in some embodiments, release of a sanitizing agent 1020 from a sanitizing surface 1012 may be decreased by selecting a sanitizing agent 1020 and a sanitizing surface 1012 that are chemically attracted to each other. Examples of such embodiments include those where a hydrophilic sanitizing agent is paired with a hydrophilic sanitizing surface or where a hydrophobic sanitizing agent is paired with a hydrophobic sanitizing surface. In some embodiments, release of a sanitizing agent 1020 from a sanitizing surface 1012 may be increased by selecting a sanitizing agent 1020 and a sanitizing surface 1012 that are chemically repelled from each other. Examples of such embodiments include those where a hydrophilic sanitizing agent 1020 is paired with a hydrophobic sanitizing surface 1012 or where a hydrophobic sanitizing agent 120 is paired with a hydrophilic sanitizing surface 1012. Accordingly, numerous combinations of sanitizing agents 1020 can be paired with numerous combinations of sanitizing surfaces 1012 to control release of a sanitizing agent 1020 from a sanitizing surface 1012.

In some embodiments, one or more sanitizing agents 1020 may be selected based on their physical and chemical properties to control release of the one or more sanitizing agents 1020 from a sanitizing surface 1020. In some embodiments, one or more sanitizing agents 1020 may be mixed with other substances to form mixtures having selected physical and chemical properties that control release of the one or more sanitizing agents 1020 from a sanitizing surface 1020. In some embodiments, one or more sanitizing agents 1020 may be selected based on the surface tension that they exhibit while on the sanitizing surface 1012 to control release of the one or more sanitizing agents 1020. In some embodiments, one or more sanitizing agents 1020 may be mixed with high molecular weight molecules that allow the resulting mixture to be self-siphoning to facilitate delivery of the sanitizing agents 1020 to a sanitizing surface 1012. In some embodiments, one or more sanitizing agents 1020 may be mixed with fluids that exhibit non-Newtonian characteristics to control release of the sanitizing agents 1020 from a sanitizing surface 1012. In some embodiments, the electrochemical characteristics of a sanitizing agent 1020 or a mixture that includes a sanitizing agent 1020 may be used to control release of the sanitizing agent 120 from a sanitizing surface 1012.

In some embodiments, substances which are known to exhibit good release properties may be combined with porous and/or permeable layers to provide for movement of one or more sanitizing agents 1020 through the porous and/or permeable layers to the sanitizing surface 1012 where they are released. Movement of the one or more sanitizing agents 1020 through the porous and/or permeable layers may occur through capillary action, wicking, use of propellants, or numerous other modalities. For example, in some embodiments, a glove 1000 may include a porous layer that is adhered to a control layer and a release layer that is adhered to the control layer. The release layer may serve as the sanitizing surface 1012 from which one or more sanitizing agents 1020 may be released. The porous layer may include an open-celled thermosetting polymer foam that may optionally be internally reinforced. In some embodiments, the porous layer may have high compatibility with, and wettability by, the one or more sanitizing agents 1020 and have high liquid holding capacity to provide for smooth substantially continuous delivery of the one or more sanitizing agents 1020. In some embodiments, the control layer may include a porous polytetrafluoroethylene film in which the pores contain a mixture of silicone oil and silicone rubber. In some embodiments, the release layer may include a porous polytetrafluoroethylene film. Methods to create such films are known (i.e., U.S. Pat. No.: 5,779,795; herein incorporated by reference).

At embodiment 1310, the one or more sanitizing agents that are integrally associated with the one or more sanitizing layers and that are substantially freely releasable from the one or more substantially continually sanitizing surfaces may include one or more sanitizing agents that include, but are not limited to, an antibacterial agent, an antiviral agent, an antifungal agent, a biocidal agent, an antiwetting agent, a wetting agent, or an antibiotic agent. Examples of such sanitizing agents 1020 are known and have been described (i.e., The Merck Index: An Encyclopedia of Chemicals, Drugs, and Biologicals, Whitehouse Station, N.J., 13^{th} Edition, 2001).

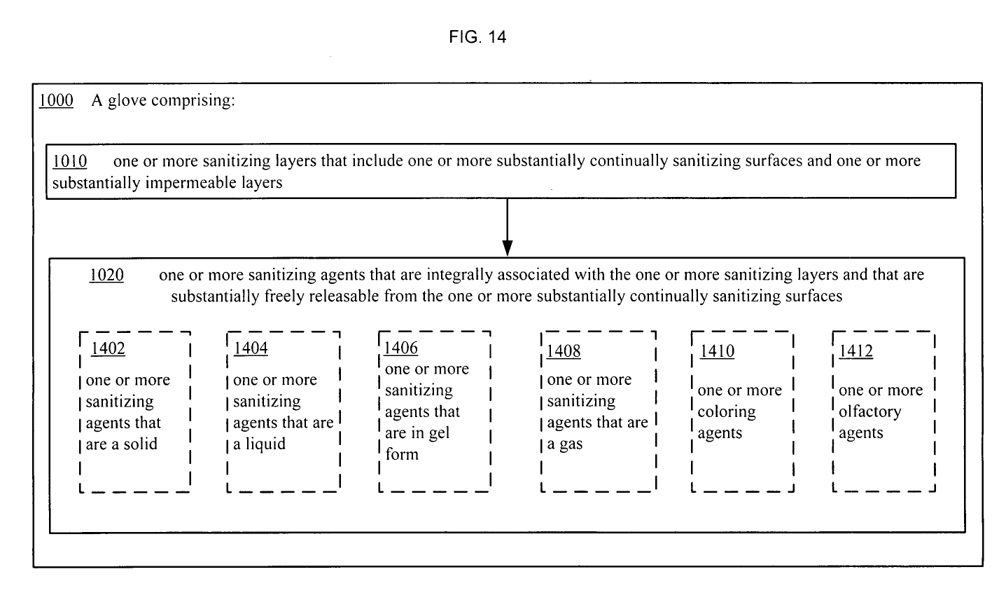

FIG. 14 illustrates alternative embodiments of the glove 1000 of FIG. 10. FIG. 14 illustrates example embodiments of the one or more sanitizing agents that are integrally associated with the one or more sanitizing layers and that are substantially freely releasable from the one or more substantially continually sanitizing surfaces 1020. Additional embodiments may include embodiment 1402, embodiment 1404, embodiment 1406, embodiment 1408, embodiment 1410, and/or embodiment 1412.

At embodiment 1402, the one or more sanitizing agents that are integrally associated with the one or more sanitizing layers and that are substantially freely releasable from the one or more substantially continually sanitizing surfaces may include one or more sanitizing agents that are a solid.

Numerous solid sanitizing agents 1020 are known and have been described herein. Such sanitizing agents 1020 include, but are not limited to, complexes of polyvinylpyrrolidone (PVP) and H₂O₂, 1,4-dichlorobenzene, complexes of iodine, and the like (i.e., U.S. Pat. No.: 5,008,106; hereby incorporated by reference). In some embodiments, one or more solid sanitizing agents 1020 that dissolve upon contact with a liquid such as water may be associated with a sanitizing layer 1010. Examples of such sanitizing agents 1020 include, but are not limited to, sodium dodecyl sulfate, lithium sulfate, lauric acid, and salts thereof. In some embodiments, such sanitizing agents 1020 may be used at one or more concentrations and under conditions where the sanitizing agents 1020 will exhibit antiviral activity. In some embodiments, such sanitizing agents 1020 may be used at one or more concentrations and under conditions where the sanitizing agents 1020 will exhibit a spermicidal activity. Accordingly, in some embodiments, substances are provided that may be used in the manufacture of prophylactic devices (i.e., U.S. Pat. No.: 6,192,887; herein incorporated by reference).

At embodiment 1404, the one or more sanitizing agents that are integrally associated with the one or more sanitizing layers and that are substantially freely releasable from the one or more substantially continually sanitizing surfaces may include one or more sanitizing agents that are a liquid.

Numerous liquid sanitizing agents 1020 are known and have been described herein. Such sanitizing agents 1020 include, but are not limited to, hydrogen peroxide, alcohols, detergents, solutions of sanitizing agents 120, and the like (i.e., U.S. Pat. Nos.: 4,642,165; 4,744,951; 5,008,106; herein incorporated by reference). In some embodiments, one or more sanitizing agents 1020 may form a solution that exhibits sanitizing activity. For example, in some embodiments, a sanitizing agent 1020 may include a water soluble salt (i.e., sodium carbonate) in combination with an anti-microbial agent (i.e., sulfur) that dissolves upon exposure to water and forms an alkaline solution that dissolves some sulfur that kills bacteria (i.e., U.S. Pat. No.: 2,216,333; herein incorporated by reference).

TABLE II
Examples of liquid sanitizing agents 1020 that
may be included in gloves 1000

| | Known Minimum | |
|---|---|---|
| | Concentration by | Known Minimum |
| | Volume for Immediately | Concentration by |
| | Sterilizing Human | Volume for |
| Chemical | Immunodeficiency | Immediately Sterilizing |
| Compound | Virus (HIV-1) | Hepatitis B Virus |
| Ethyl Alcohol | 50% | 80% |
| Isopropyl Alcohol | 30% | 70% |
| NP-40 (ethylphenyl- | 1% | — |
| polyethylene glycol) | | |
| Hydrogen Peroxide | 0.3% | — |
| Household Bleach | 0.1% | 10% |

At embodiment 1406, the one or more sanitizing agents that are integrally associated with the one or more sanitizing layers and that are substantially freely releasable from the one or more substantially continually sanitizing surfaces may include one or more sanitizing agents that are in gel form.

In some embodiments, one or more sanitizing agents 1020 may be prepared in gel form. The viscosity of such gels may be varied to control the rate at which the sanitizing agents 1020 are released from one or more sanitizing surfaces 1012. For example, in some embodiments, one or more sanitizing agents 1020 may be prepared in gel form having very low viscosity to provide for rapid flow through a porous layer 1104 and release from a sanitizing surface while in other embodiments, one or more sanitizing agents 1020 may be prepared in gel form having very high viscosity to provide for slow flow through a porous layer 1104 and slower release from a sanitizing layer 1010. In some embodiments, one or more sanitizing agents 1020 may be prepared in gel form to alter the vapor pressure associated with the sanitizing agent 1020.

Gels may be made by combining one or more sanitizing agents 1020 with one or more viscosity-modifying substances. Examples of such substances include, but are not limited to, xantham gum, gum acacia, gum tragacanth, agar, glycyrrhiza, polyvinylpyrrolidone polymers having an average molecular weight between about 500 to about 5000 grams/mole, cross-linked polyvinylpyrrolidone polymers, sodium alginate NF, pectin NF from citrus fruit or apple pomace, other plant gums, theobroma oil (also known as cacao butter or cocoa butter), cellulose, methyl cellulose (Methocel, trademark of Dow Chemical Co.) carboxymethylcellulose (CMC) sodium, hydroxyethyl cellulose (Cellosize, trademark of The Carbide and Carbon Chemicals Corp.), hydroxpropylmethylcelluloses designated Methocel 60, HG, Methocel HG65, Methocel HG70, Methocel HG90 (wherein the number refers to the approximate gel point of a 2 percent solution), other alkylated celluloses including ethylcellulose, hydroxyethyl cellulose, propylcellulose, microcrystalline cellulose (Avicel PH, trademark of FMC Corporation, Philadelphia, Pa.), other suitable chemically-modified celluloses, glycerol, propylene glycol, pyroxylin, polyethylene glycols of between about 150 to more than about 6000 molecular weight, polyethylene glycol 400, polyethylene glycol 4000, polyethylene glycol 6000, gelatin A, gelatin B, glycinerated gelatin, wool fat, beeswax, White petrolatum USP, Petrolatum NF (Petroleum Jelly with a melting point of 42-60° C.), Plastibase (tradename “Plastibase” from E.R. Squibb & Co., also called Jelene: a combination of mineral oils and heavy hydrocarbon waxes with a molecular weight of about 1300), anhydrous lanolin USP, microcrystalline wax, cholesterol, white wax, hard paraffin wax, yellow soft paraffin wax, white soft paraffin wax, sodium lauryl sulfate, stearyl alcohol, carbowax polyethylene glycol 1000, carbowax polyethylene glycol 1500, carbowax polyethylene glycol 1540, carbowax polyethylene glycol 4000, carbowax polyethylene glycol 6000, dimethicones including those more than 1000 centistokes in viscosity, simethicone, dtmethylpolysiloxane, perfluropolymethylisopropyl ethers of 1000 to more than 6600 molecular weight, starch, other alkylated starches, other chemically-modified starches, bentonite USP, sodium bentonite, potassium bentonite, calcium bentonite, magnesium bentonite, hydrogen bentonite, Voloclay bentonite (a combination of sodium bentonite, potassium bentonite, calcium bentonite, magnesium bentonite, and hydrogen bentonite), attapulgite (a hydrous magnesium aluminum silicate that is heat-activated), Veegum (a colloidal magnesium aluminum silicate), carbopol 934 at neutral pH (a trademark acidic polymer of B.F. Goodrich Chemical Co.), benzoinated lard, guar gum, agar, pulverized natural sponge, potato starch, corn starch, other vegetable starches, other plant cellulose fibers, other plant or mineral fibers or polymers, other synthetic polymers, and mixtures thereof.

At embodiment 1408, the one or more sanitizing agents that are integrally associated with the one or more sanitizing layers and that are substantially freely releasable from the one or more substantially continually sanitizing surfaces may include one or more sanitizing agents that are a gas.

In some embodiments, a gas may include a vapor. In some embodiments, hydrogen peroxide may be released from the one or more substantially continually sanitizing surfaces 1012. Hydrogen peroxide may be generated from peroxide compounds. Examples of such peroxide compounds include, but are not limited to, hydrogen peroxide, urea hydrogen peroxide, benzoyl peroxide, succinyl peroxide, barium peroxide, calcium peroxide, magnesium peroxide, sodium peroxide, strontium peroxide, zinc peroxide, other peroxide compounds, and mixtures thereof. When exposed to fine metal powders or metal oxide catalysts such as manganese dioxide, the peroxides in aqueous solutions buffered at neutral pH or alkaline pH generally can release oxygen gas or can form hydrogen peroxide (U.S. Pat. Nos.: 5,357,636; 4,169,123; 4,169,124; 4,643,876; 4,943,414; herein incorporated by reference).

In some embodiments, halogens and halogenated compounds may be reacted in aqueous or aqueous/alcoholic solutions with an acid to release sanitizing agents 1020 in soluble and gaseous forms. Examples of such halogens include fluorine, chlorine, bromine and iodine.

At embodiment 1410, the one or more sanitizing agents that are integrally associated with the one or more sanitizing layers and that are substantially freely releasable from the one or more substantially continually sanitizing surfaces may include one or more coloring agents.

Numerous coloring agents 1410, such as pigments, dyes, and other indicators are known and have been described (i.e., U.S. Pat. Nos.: 7,101,408; 5,403,363; 4,855,413; 4,855,412; 4,774,324; 4,500,455; 4,325,870; 4,208,324; herein incorporated by reference). In some embodiments, one or more coloring agents 1410 may be released upon depletion of one or more sanitizing agents 1020. In some embodiments, such coloring agents 1410 may be released in association with one or more sanitizing agents 1020 to indicate the presence of the one or more sanitizing agents 1020. In some embodiments, coloring agents 1410 may be visible to the unaided human eye. In some embodiments, coloring agents 1410 may be invisible to the unaided human eye and may be detected through use of instrumentation. In some embodiments, the presence or absence of one or more coloring agents 1410 may be visible as a color change. In some embodiments, the presence or absence of one or more coloring agents 1410 may be discernible through substantially any change visible to the unaided human eye. For example, in some embodiments, one or more coloring agents 1410 may be iridescent.

At embodiment 1412, the one or more sanitizing agents that are integrally associated with the one or more sanitizing layers and that are substantially freely releasable from the one or more substantially continually sanitizing surfaces may include one or more olfactory agents.

In some embodiments, a sanitizing agent 1020 may include a chemical odor or odorant that is capable of causing either a pleasant or an unpleasant (malodorous) smell. Numerous odorants may be utilized as olfactory agents 1412. Examples of such odorants include, but are not limited to, aromatic oils, perfumes, esters, ketones, aldehydes, organic acids, sulfides, amines, flower extracts, plant extracts, animal extracts, mineral extracts, or any other suitable chemical. For example a sanitizing agent 1020 may include a pleasant scented volatile oil such as peppermint oil, menthol, oil of wintergreen, lemon oil and the like, or an unpleasant odor such as pyridine, putrescene, ammonia, vinegar, formaldehyde, and the like. In some embodiments, an odor will be present when the sanitizing agent 1020 is present. In some embodiments, an odor will appear when the sanitizing agent 1020 is not present. Accordingly, odor can act to indicate the presence or absence of a sanitizing agent 1020.

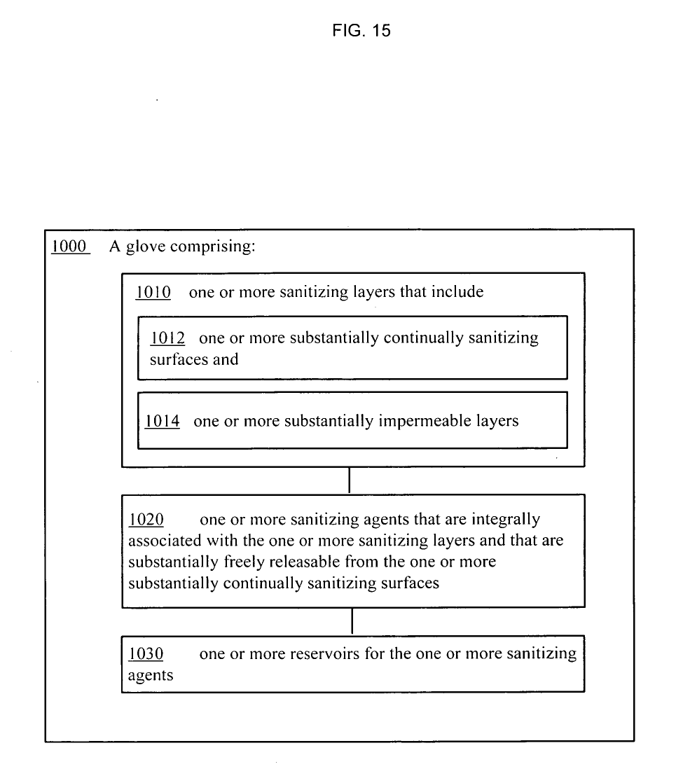

FIG. 15 represents a glove 1000 that may include one or more sanitizing layers that include one or more substantially continually sanitizing surfaces and one or more substantially impermeable layers, one or more sanitizing agents that are integrally associated with the one or more sanitizing layers and that are substantially freely releasable from the one or more substantially continually sanitizing surfaces, and one or more reservoirs for the one or more sanitizing agents.

In some embodiments, a sanitizing layer 1010 may include one or more reservoirs 1030. In some embodiments, such reservoirs 1030 may contain one type of sanitizing agent 1020. In some embodiments, such reservoirs 1030 may contain one or more types of sanitizing agents 1020. In some embodiments, such reservoirs 1030 may contain components other than sanitizing agents 1020. For example, such reservoirs 1030 may contain coloring agents 1410, olfactory agents 1412, and the like.

In some embodiments, reservoirs 1030 may be integrally associated with a sanitizing layer 1010. For example, in some embodiments, a reservoir 1030 may be contained within a sanitizing layer 1010 that opens onto the surface of the sanitizing layer 1010. In other embodiments, a sanitizing layer 1010 may include a reservoir 1030 that connects to a channel 1110 that opens onto the surface of the sanitizing layer 1010. In some embodiments, a glove 1000 may include one or more reservoirs 1030 that may be connected to one or more sanitizing layers 1010 through a connection, such as tubing. Accordingly, reservoirs 1030 may be configured in numerous geometries.

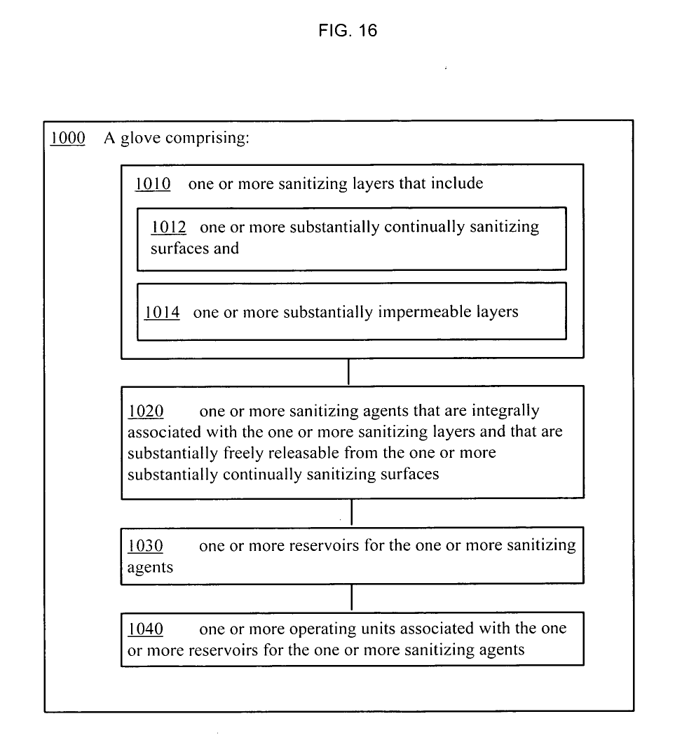

FIG. 16 represents a glove 1000 that may include one or more sanitizing layers that include one or more substantially continually sanitizing surfaces and one or more substantially impermeable layers, one or more sanitizing agents that are integrally associated with the one or more sanitizing layers and that are substantially freely releasable from the one or more substantially continually sanitizing surfaces, one or more reservoirs for the one or more sanitizing agents, and one or more operating units associated with the one or more reservoirs for the one or more sanitizing agents.

In some embodiments, one or more operating units 1040 may be associated with one or more reservoirs 1030 that may include one or more sanitizing agents 1020. In some embodiments, the one or more reservoirs 1030 may be integrally associated with the one or more sanitizing layers 1010. In some embodiments, the one or more reservoirs 1030 may be remotely associated with the one or more sanitizing layers 1010 through use of a connector, such as a tube. In some embodiments, an operating unit 1040 may control the transport of one or more sanitizing agents 1020 from one or more reservoirs 1030 to one or more sanitizing layers 110. In some embodiments, an operating unit 1040 may control the transport of one or more sanitizing agents 1020 from one or more reservoirs 1030 to one or more substantially continually sanitizing surfaces 1012. For example, in some embodiments, an operating unit 1040 may include a pump that propels one or more sanitizing agents 1020 from one or more reservoirs 1030. In some embodiments, an operating unit 1040 may include one or more valves that control the release of one or more sanitizing agents 1020 from one or more reservoirs 1030. Accordingly, numerous configurations of hardware and software may be included within an operating unit 1040 to control transport of one or more sanitizing agents 1020 from one or more reservoirs 1030. In some embodiments, one or more operating units 1040 may receive one or more signals that include one or more instructions that control the actions of the operating unit 1040. For example, in some embodiments, an operating unit 1040 may receive a signal to propel one or more sanitizing agents 1020 from one or more reservoirs 1030 to a sanitizing layer 1010 and/or to a substantially continually sanitizing surface 1012. In some embodiments, an operating unit 1040 may receive a signal to stop propelling one or more sanitizing agents 1020 from one or more reservoirs 1030 to a sanitizing layer 1010 and/or to a substantially continually sanitizing surface 1012. Accordingly, in some embodiments, one or more operating units 1040 may be programmed to cause a surface to be sanitized according to virtually any set of instructions. In some embodiments, the one or more operating units 1040 may include control features that provide for user interaction with the operating units 1040 through one or more user interfaces 1050. In some embodiments, user interaction may occur through direct interaction with an operating unit 1040 through use of one or more user interfaces 1050 that include, but are not limited to, switches, buttons, touch pads, levers, and the like). In some embodiments, user interaction may occur through indirect interaction with an operating unit 1040 through one or more user interfaces 1050 that include, but are not limited to, a wireless connection, an internet connection, a hardwired connection, and the like. In some embodiments, one or more operating units 1040 may be able to interact with additional devices. For example, in some embodiments, an operating unit 1040 may receive a signal from another device to sanitize a surface when glove 1000 associated with the operating unit 1040 enters or leaves an area. One example of such an instance would be when a glove 1000 having a sanitizing surface enters into an operating room, an associated operating unit 1040 may receive one or more signals to sanitize the glove 1000. Accordingly, numerous other configurations and control devices may be associated with an operating unit 1040. Such configurations and devices include those described within U.S. patent application Ser. No. 11/414,743, entitled METHODS AND SYSTEMS FOR MONITORING STERILIZATION STATUS; and U.S. patent application Ser. No. 11/440,460, entitled METHODS AND SYSTEMS FOR STERILIZATION; herein incorporated by reference. Accordingly, in some embodiments, a glove 1000 may include a sterilization indicator as described as described within U.S. patent application Ser. No. 11/440,460.

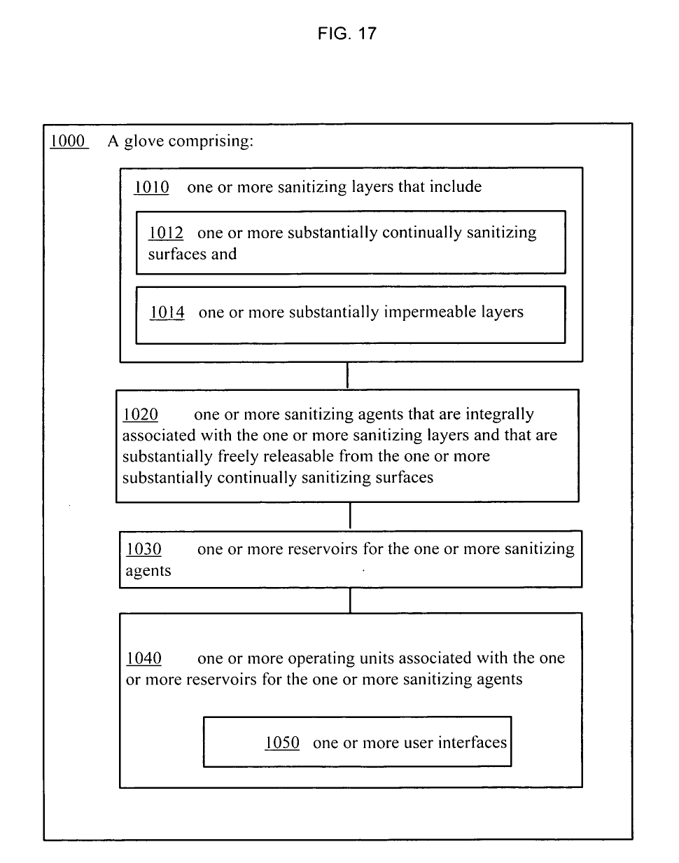

FIG. 17 represents a glove 1000 that may include one or more sanitizing layers that include one or more substantially continually sanitizing surfaces and one or more substantially impermeable layers, one or more sanitizing agents that are integrally associated with the one or more sanitizing layers and that are substantially freely releasable from the one or more substantially continually sanitizing surfaces, one or more reservoirs for the one or more sanitizing agents, one or more operating units associated with the one or more reservoirs for the one or more sanitizing agents, and the one or more operating units provide one or more user interfaces.

In some embodiments, one or more users may interact with one or more operating units 1040 through one or more user interfaces 1050. Such user interaction can include, but is not limited to, controlling an operating unit 1040 with regard to parameters associated with the operating unit 1040, a sanitizing layer 1010, and/or a sanitizing agent 1020. Parameters may include, but are not limited to, the time, place, duration, intensity, priority, and/or identity of one or more sanitizing agents 1020 that are used to sanitize a sanitizing layer 1010.

User interaction may occur directly or indirectly. For example, in some embodiments, a user may directly interact with an operating unit 1040 through use of one or more user interfaces 1050 that include, but are not limited to, switches, levers, buttons, a keyboard, a touchpad, and the like. In some embodiments, a user may interact with an operating unit 1040 indirectly by transmitting one or more signals from one or more user interfaces 1050 that are received by an operating unit 1040 that control transport of a sanitizing agent 1020 to a sanitizing layer 1010. In some embodiments, a user is human. In some embodiments, a user is not human.

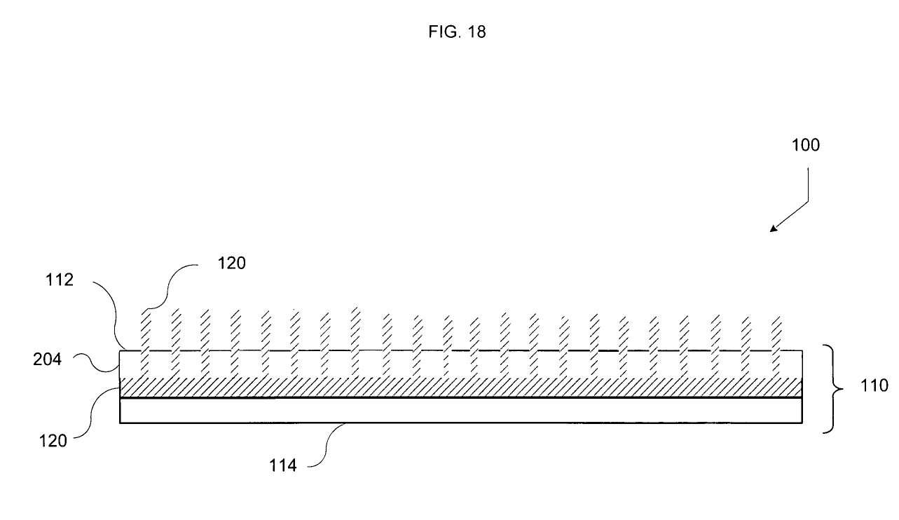

FIG. 18 represents a cross-sectional view of an embodiment of a sanitizing layer 110 that may be included in material 100. A sanitizing agent 120 is shown as positioned between a substantially impermeable layer 114 and a substantially porous layer 204. The sanitizing agent 120 is illustrated passing through the substantially porous layer 204 and onto the substantially continually sanitizing surface 112 of the sanitizing layer 110 of material 100. The embodiment illustrated in FIG. 18 may be used to construct numerous articles, such as gloves 1000.

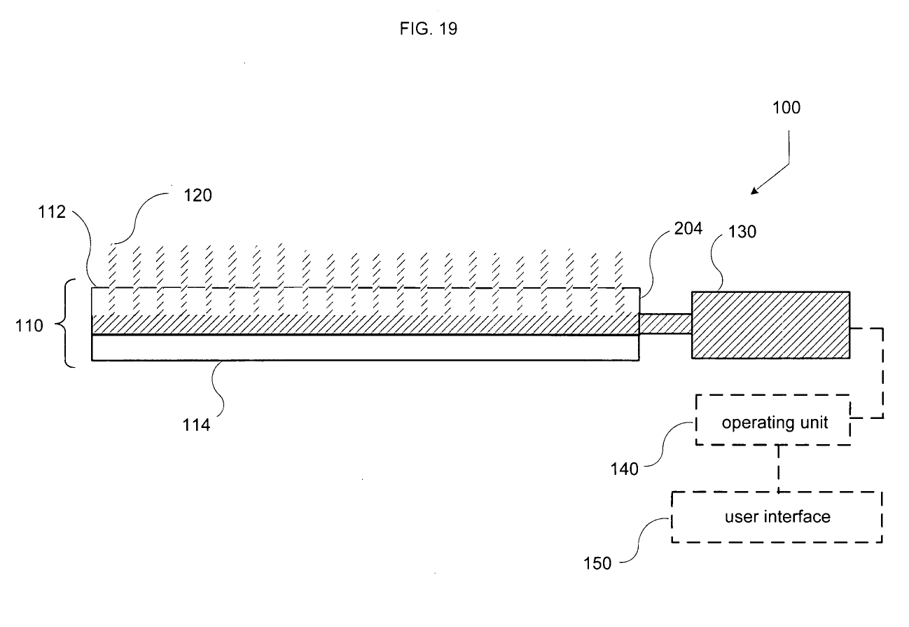

FIG. 19 represents a cross-sectional view of an embodiment of a sanitizing layer 110 that may be included in material 100. A sanitizing agent 120 is shown as positioned between a substantially impermeable layer 114 and a substantially porous layer 204. The sanitizing agent 120 is illustrated passing through the substantially porous layer 204 and onto the substantially continually sanitizing surface 112 of the sanitizing layer 110 of material 100. Also shown is a reservoir 130 that may include one or more sanitizing agents 120 that are able to be transported to the sanitizing layer 110. In some embodiments, one or more operating units 140 may be associated with one or more reservoirs 130. In some embodiments, the one or more operating units 140 may provide a user interface 150. The embodiment illustrated in FIG. 19 may be used to construct numerous articles, such as gloves 1000.

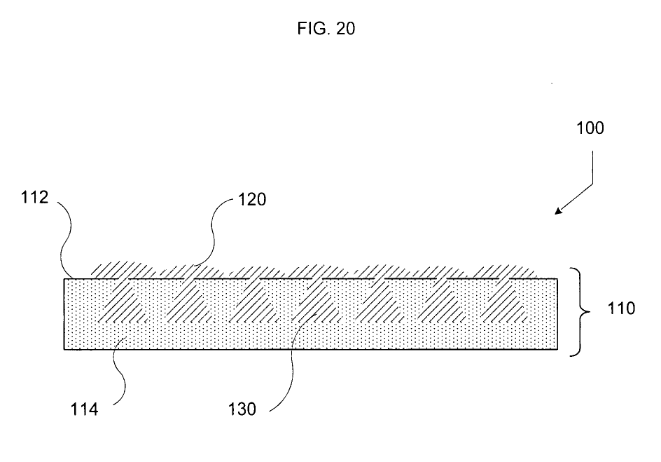

FIG. 20 represents a cross-sectional view of an embodiment of a sanitizing layer 110 that may be included in material 100. A sanitizing agent 120 is shown as being contained within numerous reservoirs 130 that are included within the sanitizing layer 110. The sanitizing agent 120 is illustrated passing through the pores onto the substantially continually sanitizing surface 112 of the sanitizing layer 110 of material 100. A substantially impermeable layer 114 is also indicated. The embodiment illustrated in FIG. 20 may be used to construct numerous articles, such as gloves 1000.

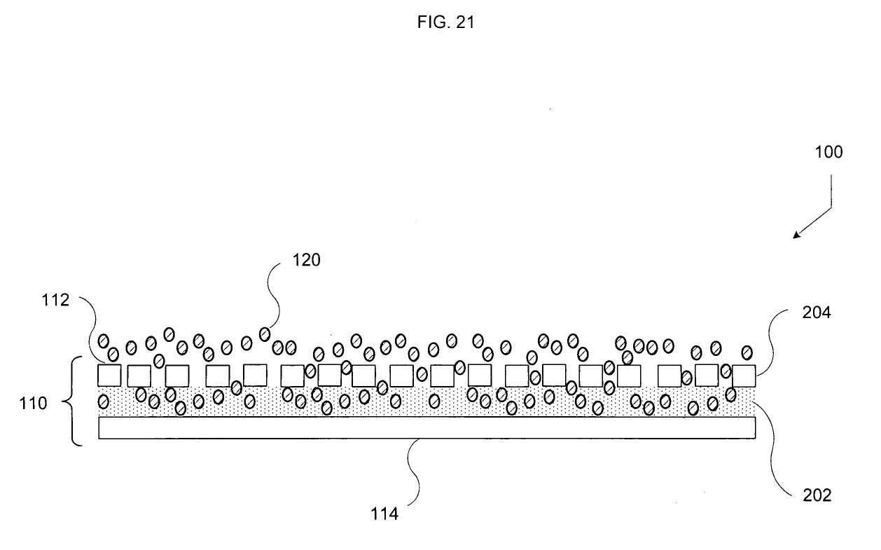

FIG. 21 represents a cross-sectional view of an embodiment of a sanitizing layer 110 that may be included in material 100. A liquid sanitizing agent 120 is shown that is contained within a substantially permeable layer 202 that is positioned between a substantially porous layer 204 and a substantially impermeable layer 114. The sanitizing agent 120 is illustrated as vaporizing and passing through the substantially porous layer 204 onto the substantially continually sanitizing surface 112 of the sanitizing layer 110 of material 100. The embodiment illustrated in FIG. 21 may be used to construct numerous articles, such as gloves 1000.

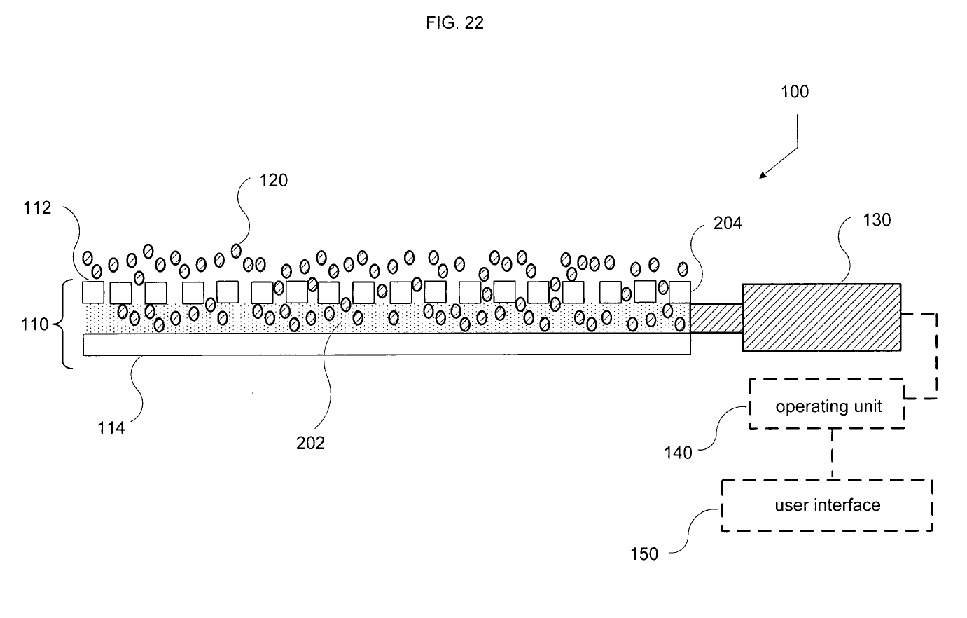

FIG. 22 represents a cross-sectional view of an embodiment of a sanitizing layer 110 that may be included in material 100. A liquid sanitizing agent 120 is shown that is contained within a substantially permeable layer 202 that is positioned between a substantially porous layer 204 and a substantially impermeable layer 114. The sanitizing agent 120 is illustrated as vaporizing and passing through the substantially porous layer 204 onto the substantially continually sanitizing surface 112 of the sanitizing layer 110 of material 100. Also shown is a reservoir 130 that may include one or more sanitizing agents 120 that are able to be transported to the sanitizing layer 110. In some embodiments, one or more operating units 140 may be associated with one or more reservoirs 130. In some embodiments, the one or more operating units 140 may provide a user interface 150. The embodiment illustrated in FIG. 22 may be used to construct numerous articles, such as gloves 1000.

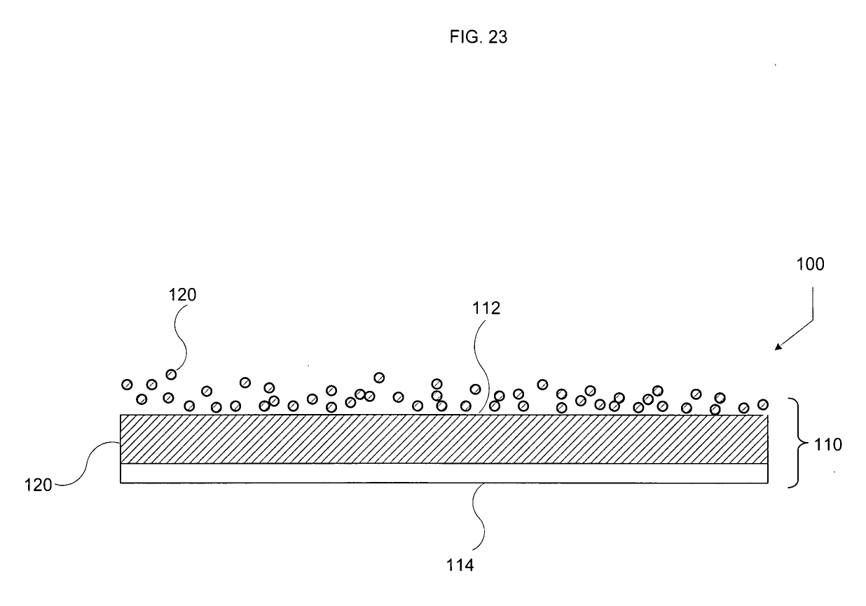

FIG. 23 represents a cross-sectional view of an embodiment of a sanitizing layer 110 that may be included in material 100. A solid sanitizing agent 120 is shown that is layered onto a substantially impermeable layer 114. The sanitizing agent 120 is illustrated as sublimating from the substantially continually sanitizing surface 112 of the sanitizing layer 110 of material 100. The embodiment illustrated in FIG. 22 may be used to construct numerous articles, such as gloves 1000.

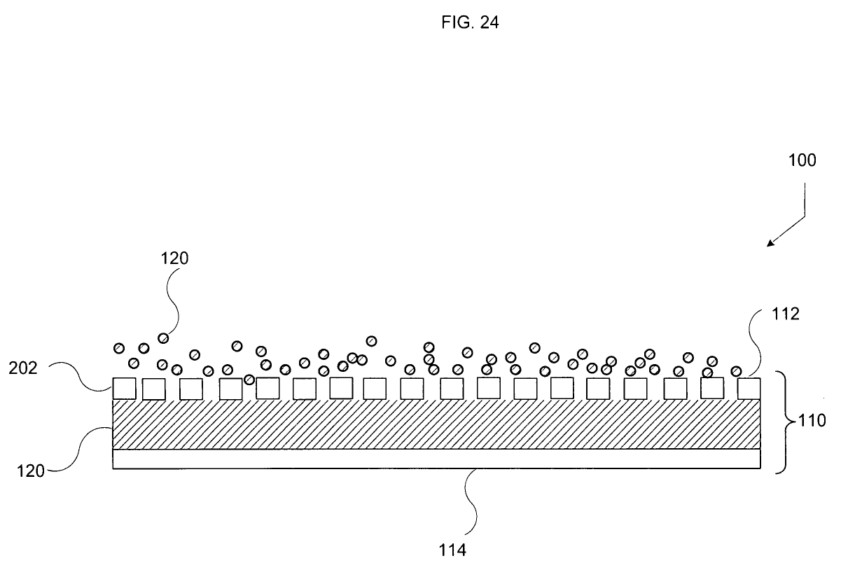

FIG. 24 represents a cross-sectional view of an embodiment of a sanitizing layer 110 that may be included in material 100. A solid sanitizing agent 120 is shown that is positioned between a substantially impermeable layer 114 and a substantially permeable layer 202. The sanitizing agent 120 is shown sublimating and passing through the substantially permeable layer 202. Accordingly, the sanitizing agent 120 is substantially freely releasable from the one or more substantially continually sanitizing surfaces 112 of material 100. The embodiment illustrated in FIG. 24 may be used to construct numerous articles, such as gloves 1000.

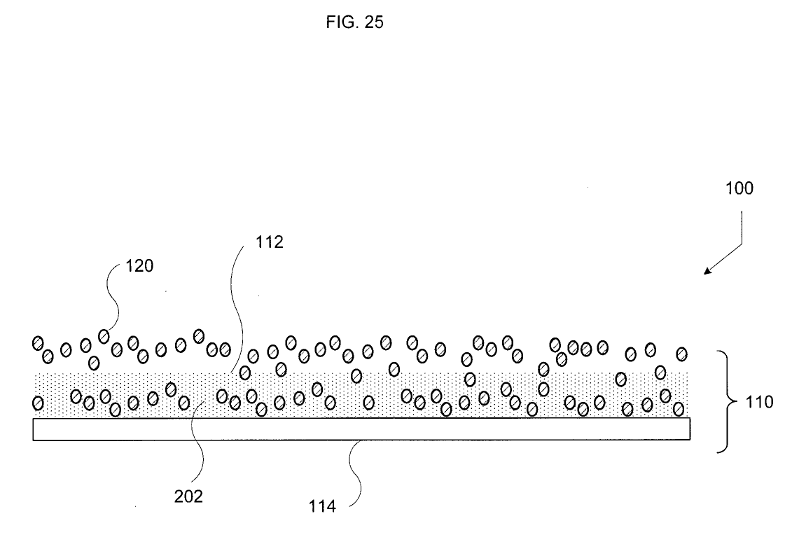

FIG. 25 represents a cross-sectional view of an embodiment of a sanitizing layer 110 that may be included in material 100. A liquid sanitizing agent 120 is shown that is contained within a substantially permeable layer 202 that is layered onto a substantially impermeable layer 114. The sanitizing agent 120 is illustrated as being present within the substantially permeable layer 202 and vaporizing from the substantially continually sanitizing surface 112 of the sanitizing layer 110 of material 100. The embodiment illustrated in FIG. 25 may be used to construct numerous articles, such as gloves 1000.

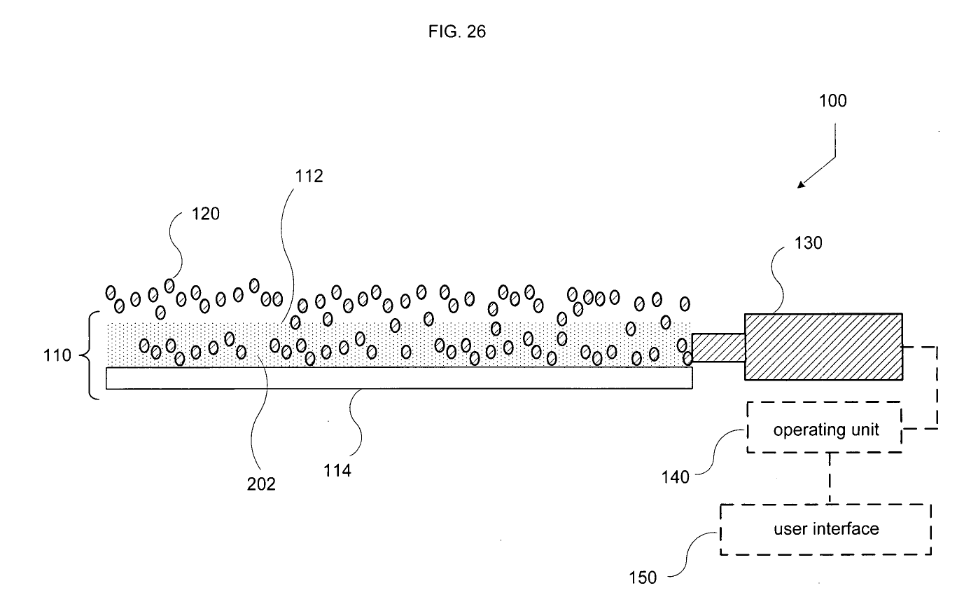

FIG. 26 represents a cross-sectional view of an embodiment of a sanitizing layer 110 that may be included in material 100. A liquid sanitizing agent 120 is shown that is contained within a substantially permeable layer 202 that is layered onto a substantially impermeable layer 114. The sanitizing agent 120 is illustrated as being present within the substantially permeable layer 202 and vaporizing from the substantially continually sanitizing surface 112 of the sanitizing layer 110 of material 100. Also shown is a reservoir 130 that may include one or more sanitizing agents 120 that are able to be transported to the sanitizing layer 110. In some embodiments, one or more operating units 140 may be associated with one or more reservoirs 130. In some embodiments, the one or more operating units 140 may provide a user interface 150. The embodiment illustrated in FIG. 26 may be used to construct numerous articles, such as gloves 1000.

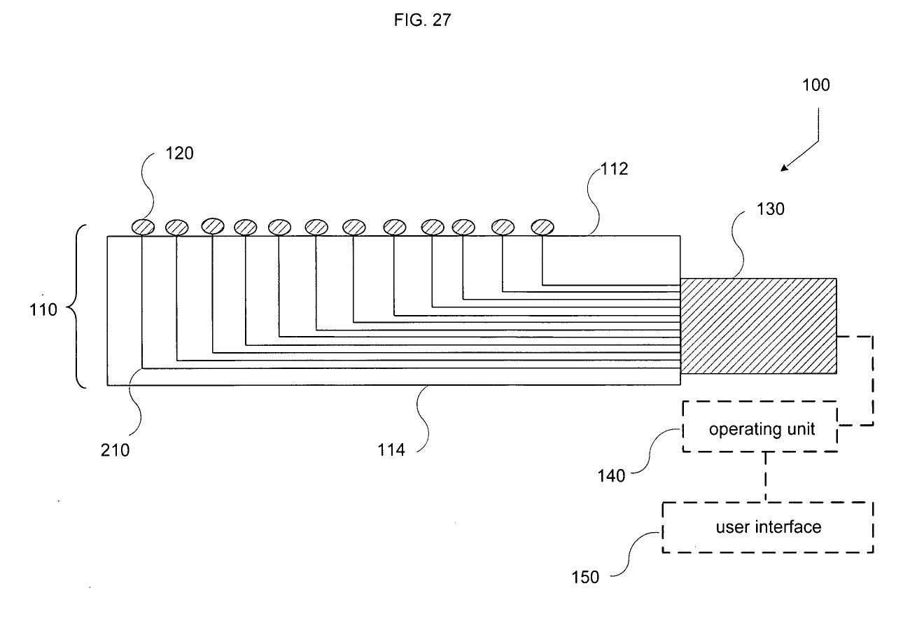

FIG. 27 represents a cross-sectional view of an embodiment of a sanitizing layer 110 that may be included in material 100. A sanitizing layer 110 is shown that includes a plurality of channels 210 through which a sanitizing agent 120 can flow to be released from the substantially continually sanitizing surface 112 of the sanitizing layer 110 of material 100. Also shown is a reservoir 130 that may include one or more sanitizing agents 120 that are able to be transported to the sanitizing layer 110. In some embodiments, one or more operating units 140 may be associated with one or more reservoirs 130. In some embodiments, the one or more operating units 140 may provide a user interface 150. The embodiment illustrated in FIG. 27 may be used to construct numerous articles, such as gloves 1000.

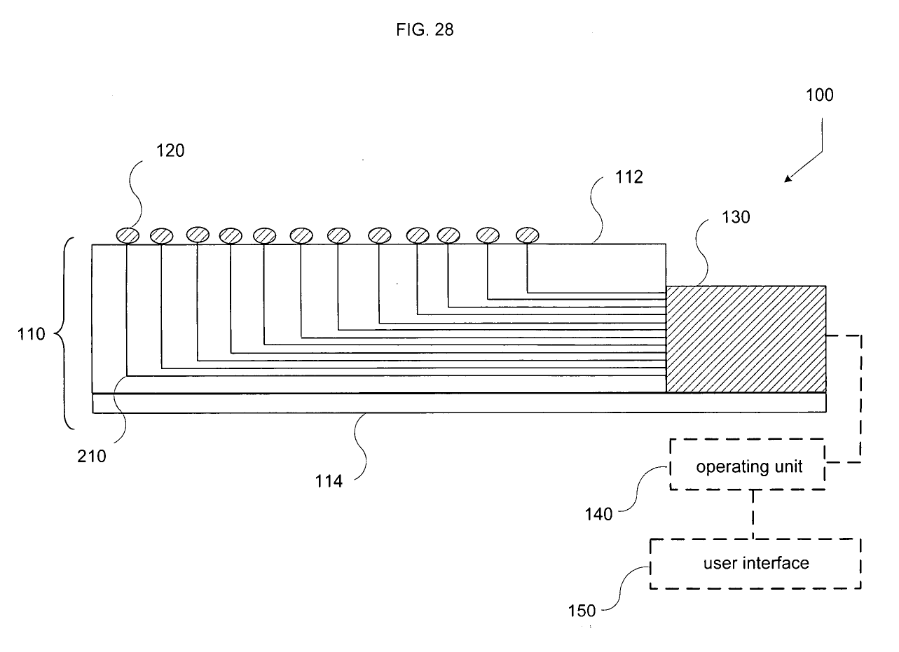

FIG. 28 represents a cross-sectional view of an embodiment of a sanitizing layer 110 that may be included in material 100. A sanitizing layer 110 is shown that includes a substantially impermeable layer 114 and another layer that includes a plurality of channels 210 through which a sanitizing agent 120 can flow to be released from the substantially continually sanitizing surface 112 of the sanitizing layer 110 of material 100. Also shown is a reservoir 130 that may include one or more sanitizing agents 120 that are able to be transported to the sanitizing layer 110. In some embodiments, one or more operating units 140 may be associated with one or more reservoirs 130. In some embodiments, the one or more operating units 140 may provide a user interface 150. The embodiment illustrated in FIG. 28 may be used to construct numerous articles, such as gloves 1000.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various embodiments described herein can be implemented, individually and/or collectively, by various types of electromechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electromechanical systems include, but are not limited to, a variety of consumer electronics systems, as well as other systems such as motorized transport systems, factory automation systems, security systems, and communication/computing systems. Those skilled in the art will recognize that electromechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, hovercraft, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a voice-over IP system, etc.), (f) a business entity (e.g., an Internet Service Provider (ISP) entity such as Comcast Cable, Quest, Southwestern Bell, etc), or (g) a wired/wireless services entity such as Sprint, Cingular, Nextel, etc.), etc.

Although a user is shown/described herein as a single illustrated figure, those skilled in the art will appreciate that a user may be representative of a human user, a robotic user (e.g., computational entity), and/or substantially any combination thereof (e.g., a user may be assisted by one or more robotic agents). In addition, a user as set forth herein, although shown as a single entity may in fact be composed of two or more entities. Those skilled in the art will appreciate that, in general, the same may be said of “sender” and/or other entity-oriented terms as such terms are used herein.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to, physically mateable and/or physically interacting components and/or wirelessly interactable and/or wirelessly interacting components and/or logically interacting and/or logically interactable components.

All publications, patents and patent applications cited herein are incorporated herein by reference. The foregoing specification has been described in relation to certain embodiments thereof, and many details have been set forth for purposes of illustration, however, it will be apparent to those skilled in the art that the invention is susceptible to additional embodiments and that certain of the details described herein may be varied considerably without departing from the basic principles of the invention.

What is claimed is:

1. A material comprising:
one or more sanitizing layers that include one or more substantially continually sanitizing surfaces and one or more substantially impermeable layers; and
one or more sanitizing agents that are integrally associated with the one or more sanitizing layers and that are substantially freely releasable from the one or more substantially continually sanitizing surfaces.

2. The material according to claim 1, wherein the one or more sanitizing layers that include one or more substantially continually sanitizing surfaces and one or more substantially impermeable layers comprise:
one or more substantially permeable layers.

3.-5. (canceled)

6. The material according to claim 1, wherein the one or more sanitizing layers that include one or more substantially continually sanitizing surfaces and one or more substantially impermeable layers comprise:
one or more channels that open onto the one or more substantially continually sanitizing surfaces.

7. The material according to claim 1, wherein the one or more sanitizing layers that include one or more substantially continually sanitizing surfaces and one or more substantially impermeable layers comprise:
one or more reservoirs for the one or more sanitizing agents.

8.-13. (canceled)

14. The material according to claim 1, wherein the one or more sanitizing agents that are integrally associated with the one or more sanitizing layers and that are substantially freely releasable from the one or more substantially continually sanitizing surfaces comprise:
one or more sanitizing agents that are substantially freely releasable from the one or more substantially continually sanitizing surfaces through sublimation.

15. The material according to claim 1, wherein the one or more sanitizing agents that are integrally associated with the one or more sanitizing layers and that are substantially freely releasable from the one or more substantially continually sanitizing surfaces comprise:
one or more sanitizing agents that are substantially freely releasable from the one or more substantially continually sanitizing surfaces through physical dissociation.

16.-20. (canceled)

21. The material according to claim 1, wherein the one or more sanitizing agents that are integrally associated with the one or more sanitizing layers and that are substantially freely releasable from the one or more substantially continually sanitizing surfaces comprise:
one or more sanitizing agents that are a gas.

22. The material according to claim 1, wherein the one or more sanitizing agents that are integrally associated with the one or more sanitizing layers and that are substantially freely releasable from the one or more substantially continually sanitizing surfaces comprise:
one or more coloring agents.

23. The material according to claim 1, wherein the one or more sanitizing agents that are integrally associated with the one or more sanitizing layers and that are substantially freely releasable from the one or more substantially continually sanitizing surfaces comprise:
one or more olfactory agents.

24. The material according to claim 1, wherein the material can be included within a glove.

25. (canceled)

26. The material according to claim 1, further comprising one or more reservoirs for the one or more sanitizing agents.

27. The material according to claim 26, further comprising one or more operating units associated with the one or more reservoirs for the one or more sanitizing agents.

28. (canceled)

29. A glove comprising:
one or more sanitizing layers that include one or more substantially continually sanitizing surfaces and one or more substantially impermeable layers; and one or more sanitizing agents that are integrally associated with the one or more sanitizing layers and that are substantially freely releasable from the one or more substantially continually sanitizing surfaces.

30. The glove according to claim 29, wherein the one or more sanitizing layers that include one or more substantially continually sanitizing surfaces and one or more substantially impermeable layers comprise:
one or more substantially permeable layers.

31.-33. (canceled)

34. The glove according to claim 29, wherein the one or more sanitizing layers that include one or more substantially continually sanitizing surfaces and one or more substantially impermeable layers comprise:
one or more channels that open onto the one or more substantially continually sanitizing surfaces.

35. The glove according to claim 29, wherein the one or more sanitizing layers that include one or more substantially continually sanitizing surfaces and one or more substantially impermeable layers comprise:
one or more reservoirs for the one or more sanitizing agents.

36.-40. (canceled)

41. The glove according to claim 29, wherein the one or more sanitizing agents that are integrally associated with the one or more sanitizing layers and that are substantially freely releasable from the one or more substantially continually sanitizing surfaces comprise:
one or more sanitizing agents that are substantially freely releasable from the one or more substantially continually sanitizing surfaces through evaporation.

42. The glove according to claim 29, wherein the one or more sanitizing agents that are integrally associated with the one or more sanitizing layers and that are substantially freely releasable from the one or more substantially continually sanitizing surfaces comprise:
one or more sanitizing agents that are substantially freely releasable from the one or more substantially continually sanitizing surfaces through sublimation.

43. The glove according to claim 29, wherein the one or more sanitizing agents that are integrally associated with the one or more sanitizing layers and that are substantially freely releasable from the one or more substantially continually sanitizing surfaces comprise:
one or more sanitizing agents that are substantially freely releasable from the one or more substantially continually sanitizing surfaces through physical dissociation.

44.-48. (canceled)

49. The glove according to claim 29, wherein the one or more sanitizing agents that are integrally associated with the one or more sanitizing layers and that are substantially freely releasable from the one or more substantially continually sanitizing surfaces comprise:
one or more sanitizing agents that are a gas.

50. The glove according to claim 29, wherein the one or more sanitizing agents that are integrally associated with the one or more sanitizing layers and that are substantially freely releasable from the one or more substantially continually sanitizing surfaces comprise:
one or more coloring agents.

51. The glove according to claim 29, wherein the one or more sanitizing agents that are integrally associated with the one or more sanitizing layers and that are substantially freely releasable from the one or more substantially continually sanitizing surfaces comprise:
one or more olfactory agents.

52.-54. (canceled)

55. A glove comprising:
one or more sanitizing layers that include one or more substantially continually sanitizing surfaces and one or more substantially impermeable layers;
one or more sanitizing agents that are integrally associated with the one or more sanitizing layers and that are substantially freely releasable from the one or more substantially continually sanitizing surfaces; and
one or more reservoirs for the one or more sanitizing agents.

56. The glove according to claim 55, further comprising one or more operating units associated with the one or more reservoirs for the one or more sanitizing agents.
